(19) United States
(12) Patent Application Publication
(10) Pub. No.: US 2010/0081707 A1
(43) Pub. Date: Apr. 1, 2010

(54) DEVICES AND METHODS FOR DELIVERING POLYNUCLEOTIDES INTO RETINAL CELLS OF THE MACULA AND FOVEA

(76) Inventors: Robin R. Ali, London (GB); James W.B. Bainbridge, London (GB)

Correspondence Address: MORRISON & FOERSTER LLP, 755 PAGE MILL RD, PALO ALTO, CA, 94304-1018, US

(21) Appl. No.: 12/390,239

(22) Filed: Feb. 20, 2009

Related U.S. Application Data
(60) Provisional application No. 61/066,656, filed on Feb. 21, 2008.
(60) Provisional application No. 61/125,439, filed on Apr. 25, 2008.

(51) Int. Cl. A61K 31/7088; A61P 27/02; A61F 9/00; A61M 5/178; A61M 5/142

(52) U.S. Cl. 514/44 R; 604/187; 604/151

(57) ABSTRACT

Methods and systems for the delivery of polynucleotides to the subretinal space of the macula or fovea of an eye of a human are provided. The methods and systems are useful for treating ocular disorders.

### CROSS-REFERENCE TO RELATED APPLICATIONS

This application claims priority benefit to U.S. Provisional Patent Application Ser. No. 61/066,656, filed Feb. 21, 2008, the content of which is incorporated herein by reference in its entirety, and to U.S. Provisional Patent Application Ser. No. 61/125,439, filed on Apr. 25, 2008, the content of which is incorporated herein by reference in its entirety.

### BACKGROUND OF THE INVENTION

Leber's congenital amaurosis (LCA) is a term used to describe a group of recessively inherited severe infantile onset rod-cone dystrophies. (Hanein S, et al. Hum Mutat 2004; 23(4):306-17). Mutation of one of several genes, including RPE65, causes disease that involves impaired vision from birth and typically progresses to blindness in the third decade. (Lorenz B, et al. Invest Opthalmol Vis Sci 2000; 41(9):2735-42; Paunescu K, et al. Graefes Arch Clin Exp Opthalmol 2005; 243(5):417-26). There is no effective treatment. RPE65 is expressed in the retinal pigment epithelium (RPE) and encodes a 65 kD protein which is a key component of the visual cycle, a biochemical pathway which regenerates the visual pigment after exposure to light. (Hanein S, et al. Hum Mutat 2004; 23(4):306-17; Thompson D A, et al. Invest Opthalmol Vis Sci 2000:41(13):4293-9; Gu S M, et al. Nat Genet. 1997; 17(2):194-7; Marlhens F, et al. Nat Genet 1997; 17(2):139-41; Morimura H, et al. Proc Natl Acad Sci USA 1998; 95(6):3088-93; Lotery A J, et al. Arch Opthalmol 2000; 118(4):538-43; Thompson D A, et al. Dev Opthalmol 2003; 37:141-54; Redmond T M, et al. Nat Genet 1998; 20(4):344-51; Mata N L, et al. J Biol Chem 2004; 279(1):635-43; Jin M, et al. Cell 2005; 122(3):449-59; Moiseyev G, et al. Proc Natl Acad Sci USA 2005; 102(35):12413-8; Redmond T M, et al. Proc Natl Acad Sci USA 2005; 102(38):13658-63). Absence of functional RPE65 results in deficiency of 11-cis retinal, rendering photoreceptor cells unable to respond to light. Cone photoreceptor cells may have access to 11-cis-retinaldehyde chromophore via an alternative pathway that does not depend on RPE-derived RPE65. (Znoiko S L, et al. Invest Opthalmol Vis Sci 2002; 43(5):1604-9; Wu B X, et al. Invest Opthalmol Vis Sci 2004; 45(11):3857-62). This is consistent with cone-mediated vision in children with LCA, although progressive degeneration of cone photoreceptor cells ultimately results in loss of cone-mediated vision.

Although the retinal dystrophy caused by defects in RPE65 is severe, features of the disorder suggest it may respond to gene replacement therapy. There is useful visual function in childhood and retinal imaging suggests that photoreceptor cell death occurs late in the disease process. (Paunescu K, et al. Graefes Arch Clin Exp Opthalmol 2005; 243(5):417-26). Gene transfer has the potential therefore to improve visual function as well as to preserve existing vision. Gene replacement therapy has been demonstrated to improve visual function in the Swedish Briard dog, a naturally occurring animal model with mutated RPE65 (Veske A, et al. Genomics 1999; 57(1):57-61); subretinal delivery of recombinant adeno-associated virus (rAAV) vector containing the RPE65 cDNA in that model has resulted in improved retinal function and improved visual behaviour. (Acland G M, et al. Nat Genet. 2001; 28(1):92-5; Narfstrom K, et al. Invest Opthalmol Vis Sci 2003; 44(4):1663-72; Narfstrom K, et al. J Hered 2003; 94(1):31-7; Le Meur G, et al. Gene Ther 2007; 14(4):292-303; Aguirre G K, et al. PLoS Med 2007; 4(6):e230).

Gene therapy protocols for retinal diseases, such as LCA, retinitis pigmentosa, and age-related macular degeneration require the localized delivery of the vector to the cells in the retina. The cells that will be the treatment target in these diseases are either the photoreceptor cells in the retina or the cells of the RPE underlying the neurosensory retina. Delivering gene therapy vectors to these cells requires injection into the subretinal space between the retina and the RPE.

The central retina, most notably the macula and the fovea, is responsible for the most important part of vision in humans: fine vision, required for e.g. reading or recognizing faces. Amongst mammals, the macula and fovea structures are unique to primates. For successful gene therapy in humans, it is therefore especially important that the treatment targets the macula and/or fovea, as this is the area where the subject has most to gain from an improvement in vision. On the other hand, damage to this area will result in loss of central vision and potentially legal blindness. Injection of gene therapy vectors directly into the subretinal space underlying the macula and fovea could potentially damage this part of the retina. One possible source of damage is the needle passing through the macula, damaging the cells along the needle tract. A second potential cause of damage to the macula and fovea is from shear forces created by the volume, rate, or location of the injection of the vector during the initial detachment of the retina from the underlying RPE.

What is needed are methods for the safe and effective subretinal delivery of gene therapy vectors to the macula and fovea. What is further needed are gene therapy compositions and systems for treating ocular disorders such as, for example, LCA.

All patents, patent applications, and documents cited herein are incorporated by reference in their entirety.

### BRIEF SUMMARY OF THE INVENTION

In one aspect of the invention is a method for delivering a polynucleotide encoding a polypeptide or therapeutic RNA to the subretinal space of the central retina of an eye of a human, comprising the steps: (a) performing a vitrectomy on the eye; wherein the vitrectomy comprises removing at least a portion of the vitreous gel of the eye and replacing with a first fluid; wherein a vitrectomy space is created by the vitrectomy; (b) forming a bleb in the subretinal space of the eye outside the central retina by subretinal injection, whereby the bleb causes a localized retinal detachment; wherein the bleb comprises an effective amount of a vector comprising the polynucleotide; and (c) repositioning the bleb such that the bleb is in contact with the subretinal space of the central retina. In another aspect of the invention is a method for treating an ocular disorder in an eye of a human, the method comprising the steps: (a) performing a vitrectomy on the eye; wherein the vitrectomy comprises removing at least a portion of the vitreous gel of the eye and replacing with a first fluid; wherein a vitrectomy space is created by the vitrectomy; (b) forming a bleb in the subretinal space of the eye outside the central retina by subretinal injection, whereby the bleb causes a localized retinal detachment; wherein the bleb comprises an effective amount of a vector comprising a polynucleotide encoding a therapeutic polypeptide or therapeutic RNA; and (c) repositioning the bleb such that the bleb is in contact with the subretinal space of the central retina; wherein one or more cells in contact with the subretinal space of the central retina are transduced by the vector and express the therapeutic polypeptide or therapeutic RNA encoded by the polynucleotide. In another aspect of the invention is a method of transducing subretinal fovea cells in the eye of a human, wherein the method comprises: (a) performing a vitrectomy on the eye; wherein the vitrectomy comprises removing at least a portion of the vitreous gel of the eye and replacing with a first fluid; wherein a vitrectomy space is created by the vitrectomy; (b) forming a bleb in the subretinal space of the eye outside the central retina by subretinal injection, whereby the bleb causes a localized retinal detachment; wherein the bleb comprises an effective amount of a vector comprising a polynucleotide encoding a polypeptide or therapeutic RNA; and (c) repositioning the bleb such that the bleb is in contact with the subretinal space of the central retina; wherein one or more cells in contact with the subretinal space of the fovea are transduced by the vector; and wherein the method does not significantly adversely affect the central retinal function or central retinal structure of the eye. In another aspect of the invention is a vector for use in treating an ocular disorder in the eye of a human, wherein the vector is administered according to the method comprising: (a) performing a vitrectomy on the eye; wherein the vitrectomy comprises removing at least a portion of the vitreous gel of the eye and replacing with a first fluid; wherein a vitrectomy space is created by the vitrectomy; (b) forming a bleb in the subretinal space of the eye outside the central retina by subretinal injection, whereby the bleb causes a localized retinal detachment; wherein the bleb comprises an effective amount of a vector comprising a polynucleotide encoding a therapeutic polypeptide or therapeutic RNA; and wherein the therapeutic polypeptide or therapeutic RNA are useful for treatment of the ocular disorder; and (c) repositioning the bleb such that the bleb is in contact with the subretinal space of the central retina. In various embodiments of any of the above methods or vectors for use, any of the following additional embodiments may optionally be included. In some embodiments, the first fluid is saline. In some embodiments, the bleb is repositioned by means of a fluid-air exchange, wherein the fluid-air exchange comprises: creating an air manipulation space within the vitrectomy space by replacing a portion of the first fluid in the vitrectomy space with air; wherein the air manipulation space is used to reposition the bleb to the subretinal space of the central retina; and replacing the air manipulation space within the vitrectomy space with the first fluid. In some embodiments, the repositioned bleb remains in contact with the subretinal space of the central retina after replacing the air manipulation space with the first fluid. In some embodiments, the bleb is formed by a single subretinal injection. In some embodiments, the bleb is formed by a first and a second subretinal injection. In some embodiments, the first subretinal injection comprises injecting saline. In some embodiments, the first subretinal injection comprises injecting Ringer's solution. In some embodiments, the first subretinal injection comprises injecting the vector. In some embodiments, the second subretinal injection comprises injecting the vector. In some embodiments, the second subretinal injection comprises injecting saline. In some embodiments, the second subretinal injection comprises injecting Ringer's solution. In some embodiments, the bleb is formed by a first, a second, and a third subretinal injection. In some embodiments, the bleb is repositioned due to the weight of the bleb in the subretinal space. In some embodiments, the bleb is repositioned by altering the position of the human's head. In some embodiments, the portion of the retina located over the repositioned bleb is detached. In some embodiments, step (c) further comprises: wherein the repositioned bleb is in contact with the entire subretinal space of the central retina. In some embodiments, the entire retinal area of the central retina is detached. In some embodiments, the repositioned bleb in step (c) is left in situ without retinopexy or intraocular tamponade. In some embodiments, more than one blebs are formed in the subretinal space of the eye; wherein the more than one blebs are repositioned such that the more than one blebs are in contact with the subretinal space of the central retina. In some embodiments, at least about 10% of the retina is detached in step (c). In some embodiments, at least about 30% of the retina is detached in step (c). In some embodiments, at least about 50% of the retina is detached in step (c). In some embodiments, at least about 90% of the retina is detached in step (c). In some embodiments, the bleb is formed from injecting a total volume of no greater than about 3 ml. In some embodiments, the bleb is formed from injecting a total volume of no greater than about 2.5 ml. In some embodiments, the bleb is formed from injecting a total volume of no greater than about 2 ml. In some embodiments, the bleb is formed from injecting a total volume of no greater than about 1 ml. In some embodiments, the bleb is formed from injecting a total volume of at least about 0.5 ml. In some embodiments, the bleb is formed from injecting a total volume of at least about 1.0 ml. In some embodiments, the bleb is formed from injecting a total volume of at least about 1.5 ml. In some embodiments, the bleb is formed from injecting a total volume of about 0.5 ml to about 3 ml. In some embodiments, the bleb is formed from injecting a total volume of about 0.5 ml to about 2.5 ml. In some embodiments, the bleb is formed from injecting a total volume of about 0.1 ml to about 0.5 ml. In some embodiments, the vector is injected over about 15-17 minutes. In some embodiments, the vector is injected over about 17-20 minutes. In some embodiments, the vector is injected over about 20-22 minutes. In some embodiments, the vector is injected at a rate of about 35 to about 65 μl/ml. In some embodiments, the vector is injected at a rate of about 35 μl/ml. In some embodiments, the vector is injected at a rate of about 40 μl/ml. In some embodiments, the vector is injected at a rate of about 45 μl/ml. In some embodiments, the vector is injected at a rate of about 50 μl/ml. In some embodiments, the vector is injected at a rate of about 55 μl/ml. In some embodiments, the vector is injected at a rate of about 60 μl/ml. In some embodiments, the vector is injected at a rate of about 65 μl/ml. In some embodiments, one or more cells in contact with the subretinal space of the central retina are transduced by the vector and express the polypeptide or therapeutic RNA encoded by the polynucleotide. In some embodiments, one or more cells in contact with the subretinal space of the macula are transduced by the vector and express the polypeptide or therapeutic RNA encoded by the polynucleotide. In some embodiments, one or more cells in contact with the subretinal space of the fovea are transduced by the vector and express the polypeptide or therapeutic RNA encoded by the polynucleotide. In some embodiments, the one or more cells are retinal pigment epithelial cells. In some embodiments, the one or more cells are photoreceptor cells. In some embodiments, the concentration of the vector in the second fluid is about 1×10¹⁶ DRP/ml to about 1×10¹⁴ DRP/ml. In some embodiments, the concentration of the vector in the first fluid is about 1×10¹¹ DRP/ml. In some embodiments, the vector is an adeno-associated virus (AAV) vector. In some embodiments, the vector is a recombinant adeno-associated virus (AAV) vector. In some embodiments, the recombinant AAV vector is an AAV2, AAV4, AAV5, or AAV8 vector. In some embodiments, the recombinant AAV vector is a pseudotyped AAV vector or chimeric AAV vector. In some embodiments, the recombinant AAV vector comprises a mixture of AAV serotypes, pseudotypes, or chimeric vectors. In some embodiments, the vector is selected from the group consisting of adenoviral, HSV, and lentiviral vectors. In some embodiments, the vector is a lentiviral vector. In some embodiments, the lentiviral vector is selected from the group consisting of HIV-1, HIV-2, SIV, FIV and EIAV. In some embodiments, the polynucleotide is selected to replace a mutated gene known to cause retinal disease. In some embodiments, the polynucleotide comprises a sequence encoding a polypeptide selected from the group consisting of: Prph2, RPE65, MERTK, RPGR, RP2, RPGRIP, CNGA3, CNGB3, and GNAT2. In some embodiments, the polynucleotide is RPE65. In some embodiments, the polynucleotide is hRPE65. In some embodiments, the polynucleotide encodes the polypeptide RPE65. In some embodiments, the polynucleotide encodes the polypeptide hRPE65. In some embodiments, the polynucleotide comprises a sequence encoding a polypeptide selected from the group consisting of a neurotrophic factor, an anti-apoptotic factor, an anti-angiogenic factor, and an anti-inflammatory factor. In some embodiments, the polynucleotide comprises a sequence encoding a polypeptide selected from the group consisting of GDNF, CNTF, FGF2, PEDF, EPO, BCL2, BCL-X, NFκB, Endostatin, Angiostatin, sFlt, IL10, IL1-ra, TGFβ, and IL4. In some embodiments, the polynucleotide comprises a sequence encoding a therapeutic RNA. In some embodiments, the polynucleotide is under the control of a promoter sequence, and the promoter sequence is hRPE65 promoter (hRPEp). In some embodiments, the vector is AAV2/2-hRPE65p-hRPE65 (SEQ ID NO:1). In some embodiments, the bleb further comprises a therapeutic agent. In some embodiments, the therapeutic agent is a neurotrophic factor, an anti-angiogenic factor, an anti-angiogenic polynucleotide, an anti-angiogenic morpholino, or an anti-angiogenic antibody or antigen-binding fragment thereof. In some embodiments, the method does not significantly adversely affect central retinal function or central retinal structure. In some embodiments, the method is effective in treating the human's visual function. In some embodiments, visual function is assessed by microperimetry, dark-adapted perimetry, assessment of visual mobility, visual acuity, ERG, or reading assessment. In some embodiments, visual function is assessed by microperimetry, dark-adapted perimetry, or assessment of visual mobility. In some embodiments, the method comprises treating an ocular disorder, and the ocular disorder is selected from the group consisting of: autosomal recessive severe early-onset retinal degeneration (Leber's Congenital Amaurosis), congenital achromatopsia, Stargardt's disease, Best's disease, Doyne's disease, cone dystrophy, retinitis pigmentosa, X-linked retinoschisis, Usher's syndrome, atrophic age related macular degeneration, neovascular AMD, diabetic maculopathy, proliferative diabetic retinopathy (PDR), cystoid macular oedema, central serous retinopathy, retinal detachment, intra-ocular inflammation, and posterior uveitis. In some embodiments, the method comprises treating an ocular disorder, and the ocular disorder is autosomal recessive severe early-onset retinal degeneration. In some embodiments, the method results in an improvement in the human's visual function. In some embodiments, the method results in the prevention of or a slowing of the progression of decline of the human's visual function due to progression of the ocular disorder. In some embodiments, the method results in a slowing of the progression of decline of the human's visual function due to progression of the ocular disorder.

Another aspect of the invention is a method for delivering a polynucleotide encoding a polypeptide or therapeutic RNA to the subretinal space of the central retina of an eye of a human, comprising the steps: (a) performing a vitrectomy on the eye; wherein the vitrectomy comprises removing at least a portion of the vitreous gel of the eye and replacing with a first fluid; (b) administering by subretinal injection outside the central retina a second fluid to the subretinal space of the eye, whereby a fluid bleb is formed by the second fluid in the subretinal space causing a localized retinal detachment; wherein the second fluid comprises an effective amount of a vector comprising the polynucleotide; and (c) repositioning the fluid bleb such that the fluid bleb is in contact with the subretinal space of the central retina. In another aspect of the invention is a method for treating an ocular disorder in an eye of a human, the method comprising the steps: (a) performing a vitrectomy on the eye; wherein the vitrectomy comprises removing at least a portion of the vitreous gel of the eye and replacing with a first fluid; (b) administering by subretinal injection outside the central retina a second fluid to the subretinal space of the eye, whereby a fluid bleb is formed by the second fluid in the subretinal space causing a localized retinal detachment; wherein the second fluid comprises an effective amount of a vector comprising a polynucleotide encoding a therapeutic polypeptide or therapeutic RNA; and (c) repositioning the fluid bleb such that the fluid bleb is in contact with the subretinal space of the central retina; wherein one or more cells in contact with the subretinal space of the central retina are transduced by the vector and express the therapeutic polypeptide or therapeutic RNA encoded by the polynucleotide. In another aspect of the invention is a method of transducing subretinal fovea cells in the eye of a human, wherein the method comprises: (a) performing a vitrectomy on the eye; wherein the vitrectomy comprises removing at least a portion of the vitreous gel of the eye and replacing with a first fluid; (b) administering by subretinal injection outside the central retina a second fluid to the subretinal space of the eye, whereby a fluid bleb is formed by the second fluid in the subretinal space causing a localized retinal detachment; wherein the second fluid comprises an effective amount of a vector comprising a polynucleotide encoding a polypeptide or a therapeutic RNA; and (c) repositioning the fluid bleb such that the fluid bleb is in contact with the subretinal space of the central retina; wherein one or more cells in contact with the subretinal space of the fovea are transduced by the viral vector; and wherein the method does not significantly adversely affect the central retinal function or central retinal structure of the eye. In another aspect of the invention is a vector for use in treating an ocular disorder in the eye of a human, wherein the vector is administered according to the method comprising: (a) performing a vitrectomy on the eye; wherein the vitrectomy comprises removing at least a portion of the vitreous gel of the eye and replacing with a first fluid; (b) administering by subretinal injection outside the central retina a second fluid to the subretinal space of the eye, whereby a fluid bleb is formed by the second fluid in the subretinal space causing a localized retinal detachment; wherein the second fluid comprises an effective amount of a vector comprising a polynucleotide encoding a therapeutic polypeptide or therapeutic RNA; and wherein the therapeutic polypeptide or therapeutic RNA are useful for treatment of the ocular disorder; and (c) repositioning the fluid bleb such that the fluid bleb is in contact with the subretinal space of the central retina. In various embodiments of any of the above methods or vectors for use, any of the following additional embodiments may optionally be included. In some embodiments, the first fluid is saline. In some embodiments, the fluid bleb is repositioned by means of a fluid-air exchange, wherein the fluid-air exchange comprises replacing a portion of the first fluid in the vitreous cavity that is in contact with the surface of the retina of the eye with air. In some embodiments, the method further comprises: (d) replacing the air that is in contact with the surface of the retina of the eye with additional first fluid. In some embodiments, the repositioned fluid bleb remains in contact with the subretinal space of the central retina. In some embodiments, prior to step (b), a third fluid is administered by subretinal injection outside the central retina to the subretinal space of the eye, whereby an initial fluid bleb is formed by the third fluid in the subretinal space causing a localized retinal detachment; and wherein the second fluid is administered by subretinal injection into the initial fluid bleb formed by the third fluid to form the fluid bleb. In some embodiments, more than one fluid bleb are formed in the subretinal space of the eye; wherein the more than one fluid bleb are repositioned such that the more than one fluid blebs are in contact with the subretinal space of the central retina. In some embodiments, the second and third fluids are the same. In some embodiments, the second and third fluids are different. In some embodiments, the third fluid is Ringer's solution. In some embodiments, the third fluid is saline. In some embodiments, after step (b), a fourth fluid is administered by subretinal injection into the fluid bleb. In some embodiments, the fourth fluid is Ringer's solution. In some embodiments, the fourth fluid is saline. In some embodiments, the fluid bleb is repositioned due to the weight of the fluid bleb in the subretinal space. In some embodiments, the fluid bleb is repositioned by altering the position of the human's head. In some embodiments, the portion of the retina located over the repositioned fluid bleb is detached. In some embodiments, step (c) further comprises: wherein the repositioned fluid bleb is in contact with the entire subretinal space of the central retina. In some embodiments, the entire retinal area of the central retina is detached. In some embodiments, the repositioned fluid bleb in step (c) is left in situ without retinopexy or intraocular tamponade. In some embodiments, at least about 10% of the retina is detached in step (c). In some embodiments, at least about 30% of the retina is detached in step (c). In some embodiments, at least about 50% of the retina is detached in step (c). In some embodiments, at least about 90% of the retina is detached in step (c). In some embodiments, the amount of the second fluid administered is no greater than about 3 ml. In some embodiments, the amount of the second fluid administered is no greater than about 2.5 ml. In some embodiments, the amount of the second fluid administered is no greater than about 2 ml. In some embodiments, the amount of the second fluid administered is no greater than about 1 ml. In some embodiments, the amount of the second fluid administered is at least about 0.5 ml. In some embodiments, the amount of the second fluid administered is at least about 1.0 ml. In some embodiments, the amount of the second fluid administered is at least about 1.5 ml. In some embodiments, the amount of the second fluid administered is about 0.5 to about 3 ml. In some embodiments, the amount of the second fluid administered is about 0.5 to about 2.5 ml. In some embodiments, the amount of the third fluid administered is about 0.1 to about 0.5 ml. In some embodiments, the amount of the fourth fluid administered is no greater than about 3 ml. In some embodiments, the amount of the fourth fluid administered is no greater than about 2 ml. In some embodiments, the amount of the fourth fluid administered is no greater than about 1 ml. In some embodiments, the vector is injected over about 15-17 minutes. In some embodiments, the vector is injected over about 17-20 minutes. In some embodiments, the vector is injected over about 20-22 minutes. In some embodiments, the vector is injected at a rate of about 35 to about 65 μl/ml. In some embodiments, the vector is injected at a rate of about 35 μl/ml. In some embodiments, the vector is injected at a rate of about 40 μl/ml. In some embodiments, the vector is injected at a rate of about 45 μl/ml. In some embodiments, the vector is injected at a rate of about 50 μl/ml. In some embodiments, the vector is injected at a rate of about 55 μl/ml. In some embodiments, the vector is injected at a rate of about 60 μl/ml. In some embodiments, the vector is injected at a rate of about 65 μl/ml. In some embodiments, one or more cells in contact with the subretinal space of the central retina are transduced by the vector and express the polypeptide or therapeutic RNA encoded by the polynucleotide. In some embodiments, one or more cells in contact with the subretinal space of the macula are transduced by the vector and express the polypeptide or therapeutic RNA encoded by the polynucleotide. In some embodiments, one or more cells in contact with the subretinal space of the fovea are transduced by the vector and express the polypeptide or therapeutic RNA encoded by the polynucleotide. In some embodiments, the one or more cells are retinal pigment epithelial cells. In some embodiments, the one or more cells are photoreceptor cells. In some embodiments, the concentration of the vector in the second fluid is about 1×10¹⁶ DRP/ml to about 1×10¹⁴ DRP/ml. In some embodiments, the concentration of the vector in the first fluid is about 1×10¹¹ DRP/ml. In some embodiments, the vector is an adeno-associated virus (AAV) vector. In some embodiments, the vector is a recombinant adeno-associated virus (AAV) vector. In some embodiments, the recombinant AAV vector is an AAV2, AAV4, AAV5, or AAV8 vector. In some embodiments, the recombinant AAV vector is a pseudotyped AAV vector or chimeric AAV vector. In some embodiments, the recombinant AAV vector comprises a mixture of AAV serotypes, pseudotypes, or chimeric vectors. In some embodiments, the vector is selected from the group consisting of adenoviral, HSV, and lentiviral vectors. In some embodiments, the vector is a lentiviral vector. In some embodiments, the lentiviral vector is selected from the group consisting of HIV-1, HIV-2, SIV, FIV and EIAV. In some embodiments, the polynucleotide is selected to replace a mutated gene known to cause retinal disease. In some embodiments, the polynucleotide comprises a sequence encoding a polypeptide selected from the group consisting of: Prph2, RPE65, MERTK, RPGR, RP2, RPGRIP, CNGA3, CNGB3, and GNAT2. In some embodiments, the polynucleotide is RPE65. In some embodiments, the polynucleotide is hRPE65. In some embodiments, the polynucleotide encodes the polypeptide RPE65. In some embodiments, the polynucleotide encodes the polypeptide hRPE65. In some embodiments, the polynucleotide comprises a sequence encoding a polypeptide selected from the group consisting of a neurotrophic factor, an anti-apoptotic factor, an anti-angiogenic factor, and an anti-inflammatory factor. In some embodiments, the polynucleotide comprises a sequence encoding a polypeptide selected from the group consisting of GDNF, CNTF, FGF2, PEDF, EPO, BCL2, BCL-X, NFκB, Endostatin, Angiostatin, sFlt, IL10, IL1-ra, TGFβ, and IL4. In some embodiments, the polynucleotide comprises a sequence encoding a therapeutic RNA. In some embodiments, the polynucleotide is under the control of a promoter sequence, and the promoter sequence is hRPE65 promoter (hRPEp). In some embodiments, the vector is AAV2/2-hRPE65p-hRPE65 (SEQ ID NO:1). In some embodiments, one or more of the second, third, or fourth fluids, when present, further comprise a therapeutic agent. In some embodiments, the therapeutic agent is a neurotrophic factor, an anti-angiogenic factor, an anti-angiogenic polynucleotide, an anti-angiogenic morpholino, or an anti-angiogenic antibody or antigen-binding fragment thereof. In some embodiments, the method does not significantly adversely affect central retinal function or central retinal structure. In some embodiments, the method is effective in treating the human's visual function. In some embodiments, visual function is assessed by microperimetry, dark-adapted perimetry, assessment of visual mobility, visual acuity, ERG, or reading assessment. In some embodiments, visual function is assessed by microperimetry, dark-adapted perimetry, or assessment of visual mobility. In some embodiments, the ocular disorder is selected from the group consisting of: autosomal recessive severe early-onset retinal degeneration (Leber's Congenital Amaurosis), congenital achromatopsia, Stargardt's disease, Best's disease, Doyne's disease, cone dystrophy, retinitis pigmentosa, X-linked retinoschisis, Usher's syndrome, atrophic age related macular degeneration, neovascular AMD, diabetic maculopathy, proliferative diabetic retinopathy (PDR), cystoid macular oedema, central serous retinopathy, retinal detachment, intra-ocular inflammation, and posterior uveitis. In some embodiments, the ocular disorder is autosomal recessive severe early-onset retinal degeneration. In some embodiments, the method results in an improvement in the human's visual function. In some embodiments, the method results in the prevention of or a slowing of the progression of decline of the human's visual function due to progression of the ocular disorder. In some embodiments, the method results in a slowing of the progression of decline of the human's visual function due to progression of the ocular disorder.

In another aspect of the invention is a method for treating an ocular disorder, comprising: administering to the subretinal space of the central retina in an eye of a human in need thereof an effective amount of a vector; wherein the vector is useful for treatment of the ocular disorder when administered to the subretinal space of the central retina of the eye. In some embodiments, the vector comprises a polynucleotide encoding a therapeutic polypeptide or therapeutic RNA; and wherein the polynucleotide is under the control of a promoter suitable for expression of the therapeutic polypeptide or therapeutic RNA in one or more central retina cell types. In some embodiments, one or more cells in contact with the subretinal space of the central retina are transduced by the vector and express the therapeutic polypeptide or therapeutic RNA encoded by the polynucleotide. In some embodiments, one or more cells in contact with the subretinal space of the outer macula are transduced by the vector and express the therapeutic polypeptide or therapeutic RNA encoded by the polynucleotide. In some embodiments, one or more cells in contact with the subretinal space of the inner macula are transduced by the vector and express the therapeutic polypeptide or therapeutic RNA encoded by the polynucleotide. In some embodiments, one or more cells in contact with the subretinal space of the fovea are transduced by the vector and express the therapeutic polypeptide or therapeutic RNA encoded by the polynucleotide. In some embodiments, the one or more cells are retinal pigment epithelial cells. In some embodiments, the one or more cells are photoreceptor cells. In some embodiments, the polynucleotide encodes a therapeutic polypeptide. In some embodiments, the polynucleotide encodes a therapeutic RNA. In some embodiments, the vector is administered to the outer macula. In some embodiments, the vector is administered to the inner macula. In some embodiments, the vector is administered to the fovea. In some embodiments, the method does not significantly adversely affect central retinal function or central retinal structure. In some embodiments, the ocular disorder is selected from the group consisting of: autosomal recessive severe early-onset retinal degeneration (Leber's Congenital Amaurosis), congenital achromatopsia, Stargardt's disease, Best's disease, Doyne's disease, cone dystrophy, retinitis pigmentosa, X-linked retinoschisis, Usher's syndrome, atrophic age related macular degeneration, neovascular AMD, diabetic maculopathy, proliferative diabetic retinopathy (PDR), cystoid macular oedema, central serous retinopathy, retinal detachment, intra-ocular inflammation, and posterior uveitis. In some embodiments, the ocular disorder is autosomal recessive severe early-onset retinal degeneration. In some embodiments, the method is effective in treating the human's visual function. In some embodiments, visual function is assessed by microperimetry, dark-adapted perimetry, assessment of visual mobility, visual acuity, ERG, or reading assessment. In some embodiments, visual function is assessed by microperimetry, dark-adapted perimetry, or assessment of visual mobility. In some embodiments, the method results in an improvement in the human's visual function. In some embodiments, the method results in the prevention of or a slowing of the progression of decline of the human's visual function due to progression of the ocular disorder. In some embodiments, the method results in a slowing of the progression of decline of the human's visual function due to progression of the ocular disorder. In some embodiments, the vector is an adeno-associated virus (AAV) vector. In some embodiments, the vector is a recombinant adeno-associated virus (AAV) vector. In some embodiments, the recombinant AAV vector is an AAV2, AAV4, AAV5, or AAV8 vector. In some embodiments, the recombinant AAV vector is a pseudotyped AAV vector or chimeric AAV vector. In some embodiments, the recombinant AAV vector comprises a mixture of AAV serotypes, pseudotypes, or chimeric vectors. In some embodiments, the vector is selected from the group consisting of adenoviral, HSV, and lentiviral vectors. In some embodiments, the vector is a lentiviral vector. In some embodiments, the vector is selected from the group consisting of lentiviral HIV-1, HIV-2, SIV, FIV and EIAV. In some embodiments, the polynucleotide is selected to replace a mutated gene known to cause retinal disease. In some embodiments, the polynucleotide comprises a sequence encoding a polypeptide selected from the group consisting of: Prph2, RPE65, MERTK, RPGR, RP2, RPGRIP, CNGA3, CNGB3, and GNAT2. In some embodiments, the polynucleotide is RPE65. In some embodiments, the polynucleotide is hRPE65. In some embodiments, the polynucleotide encodes the polypeptide RPE65. In some embodiments, the polynucleotide encodes the polypeptide hRPE65. In some embodiments, the polynucleotide comprises a sequence encoding a polypeptide selected from the group consisting of a neurotrophic factor, an anti-apoptotic factor, an anti-angiogenic factor, and an anti-inflammatory factor. In some embodiments, the polynucleotide comprises a sequence encoding a polypeptide selected from the group consisting of GDNF, CNTF, FGF2, PEDF, EPO, BCL2, BCL-X, NFκB, Endostatin, Angiostatin, sFlt, IL10, IL1-ra, TGFβ, and IL4. In some embodiments, the polynucleotide comprises a sequence encoding a therapeutic RNA. In some embodiments, the polynucleotide is under the control of a promoter sequence, and the promoter sequence is hRPE65 promoter (hRPEp). In some embodiments, the vector is AAV2/2-hRPE65p-hRPE65. In some embodiments, the method further comprises administering a therapeutic agent to the subretinal space of the central retina of the eye. In some embodiments, the therapeutic agent is a neurotrophic factor, an anti-angiogenic factor, an anti-angiogenic polynucleotide, an anti-angiogenic morpholino, or an anti-angiogenic antibody or antigen-binding fragment thereof. In some embodiments, the method comprises administering to the human about 0.5 to about 3.0 ml of a suspension comprising the vector. In some embodiments, the method comprises administering to the human about 0.8 to about 3.0 ml of a suspension comprising the vector. In some embodiments, the method comprises administering to the human about 0.9 to about 3.0 ml of a suspension comprising the vector.

In another aspect of the invention is a vector for use in treating an ocular disorder in an eye of a human in need thereof, wherein the vector is useful for treatment of the ocular disorder when administered in an effective amount to the subretinal space of the central retina of the eye. Any of the vectors as described herein may be used in any of the methods as described herein.

In another aspect of the invention is a vector for use in the manufacture of a medicament for treating an ocular disorder in an eye of a human in need thereof, wherein the vector is useful for treatment of the ocular disorder when administered in an effective amount to the subretinal space of the central retina of the eye. Any of the vectors as described herein may be used in the manufacture of a medicament for use in any of the methods as described herein.

In another aspect of the invention is a system for subretinal delivery of a vector to an eye of a human, comprising: (a) a fine-bore cannula, wherein the fine bore cannula is 27 to 45 gauge; (b) a syringe; and (c) greater than about 0.8 ml of a suspension comprising an effective amount of the vector; wherein the vector comprises a polynucleotide encoding a therapeutic polypeptide or therapeutic RNA under the control of a promoter suitable for expression of the therapeutic polypeptide or therapeutic RNA in one or more central retina cell types; and wherein the vector is useful for treatment of an ocular disorder when administered to the subretinal space of the central retina of the eye. In some embodiments, the suspension is contained within the syringe. In some embodiments, the cannula is attached to the syringe. In some embodiments, the syringe is an Accurus® system syringe. In some embodiments, the system further comprises an automated injection pump. In some embodiments, the automated injection pump is activated by a foot pedal. In some embodiments, the syringe is inserted into the automated injection pump. In some embodiments, the system comprises at least about 0.9 ml of the suspension. In some embodiments, the system comprises at least about 1.0 ml of the suspension. In some embodiments, the system comprises at least about 1.5 ml of the suspension. In some embodiments, the system comprises at least about 2.0 ml of the suspension. In some embodiments, the system comprises about 0.8 to about 3.0 ml of the suspension. In some embodiments, the system comprises about 0.8 to about 2.5 ml of the suspension. In some embodiments, the system comprises about 0.8 to about 2.0 ml of the suspension. In some embodiments, the system comprises about 0.8 to about 1.5 ml of the suspension. In some embodiments, the system comprises about 0.8 to about 1.0 ml of the suspension. In some embodiments, the system comprises about 1.0 to about 3.0 ml of the suspension. In some embodiments, the system comprises about 1.0 to about 2.0 ml of the suspension. In some embodiments, the concentration of the vector in the suspension is about 1×10⁶ DRP/ml to about 1×10¹⁴ DRP/ml. In some embodiments, the concentration of the vector in the suspension is about 1×10¹¹ DRP/ml. In some embodiments, the suspension further comprises a therapeutic agent. In some embodiments, the therapeutic agent is a neurotrophic factor, an anti-angiogenic factor, an anti-angiogenic polynucleotide, an anti-angiogenic morpholino, or an anti-angiogenic antibody or antigen-binding fragment thereof. In some embodiments, the fine-bore cannula is 35-41 gauge. In some embodiments, the fine-bore cannula is 40 or 41 gauge. In some embodiments, the fine-bore cannula is 41-gauge. In some embodiments, the vector is an adeno-associated virus (AAV) vector. In some embodiments, the vector is a recombinant adeno-associated virus (AAV) vector. In some embodiments, the recombinant AAV vector is an AAV2, AAV4, AAV5, or AAV8 vector. In some embodiments, the recombinant AAV vector is a pseudotyped AAV vector or chimeric AAV vector. In some embodiments, the recombinant AAV vector comprises a mixture of AAV serotypes, pseudotypes, or chimeric vectors. In some embodiments, the vector is selected from the group consisting of adenoviral, HSV, and lentiviral vectors. In some embodiments, the vector is a lentiviral vector. In some embodiments, the lentiviral vector is selected from the group consisting of HIV-1, HIV-2, SIV, FIV and EIAV. In some embodiments, the polynucleotide is selected to replace a mutated gene known to cause retinal disease. In some embodiments, the polynucleotide comprises a sequence encoding a polypeptide selected from the group consisting of: Prph2, RPE65, MERTK, RPGR, RP2, RPGRIP, CNGA3, CNGB3, and GNAT2. In some embodiments, the polynucleotide is RPE65. In some embodiments, the polynucleotide is hRPE65. In some embodiments, the polynucleotide encodes the polypeptide RPE65. In some embodiments, the polynucleotide encodes the polypeptide hRPE65. In some embodiments, the polynucleotide comprises a sequence encoding a polypeptide selected from the group consisting of a neurotrophic factor, an anti-apoptotic factor, an anti-angiogenic factor, and an anti-inflammatory factor. In some embodiments, the polynucleotide comprises a sequence encoding a polypeptide selected from the group consisting of GDNF, CNTF, FGF2, PEDF, EPO, BCL2, BCL-X, NFκB, Endostatin, Angiostatin, sFlt, IL10, IL1-ra, TGFβ, and IL4. In some embodiments, the polynucleotide comprises a sequence encoding a therapeutic RNA. In some embodiments, the polynucleotide is under the control of a promoter sequence, and the promoter sequence is hRPE65 promoter (hRPEp). In some embodiments, the vector is AAV2/2-hRPE65p-hRPE65 (SEQ ID NO:1). In some embodiments, the vector is useful for transducing retinal pigment epithelial cells. In some embodiments, the vector is useful for transducing photoreceptor cells.

In another aspect of the invention is a system for subretinal delivery of a vector to an eye of a human, comprising: (a) a fine-bore cannula, wherein the fine bore cannula is 27 to 45 gauge; (b) a first syringe comprising a first fluid suitable for subretinal injection to the eye; and (c) a second syringe comprising a second fluid comprising an effective amount of the vector; wherein the total volume of the first and the second fluids in combination is about 0.5 to about 3.0 ml; wherein the vector comprises a polynucleotide encoding a therapeutic polypeptide or therapeutic RNA under the control of a promoter suitable for expression of the therapeutic polypeptide or therapeutic RNA in one or more central retina cell types; and wherein the vector is useful for treatment of an ocular disorder when administered to the subretinal space of the central retina of the eye. In some embodiments, the first and second syringes are Accurus® system syringes. In some embodiments, the system further comprises an automated injection pump. In some embodiments, the automated injection pump is activated by a foot pedal. In some embodiments, the total volume of the first and the second fluids in combination is about 0.8 to about 3.0 ml. In some embodiments, the total volume of the first and the second fluids in combination is about 0.9 to about 3.0 ml. In some embodiments, the total volume of the first and the second fluids in combination is about 1.0 to about 3.0 ml. In some embodiments, the volume of the first fluid is about 0.1 to about 0.5 ml. In some embodiments, the volume of the second fluid is about 0.5 to about 3.0 ml. In some embodiments, the volume of the second fluid is about 0.8 to about 3.0 ml. In some embodiments, the volume of the second fluid is about 0.9 to about 3.0 ml. In some embodiments, the volume of the second fluid is about 1.0 to about 3.0 ml. In some embodiments, the concentration of the vector in the second fluid is about 1×10⁶ DRP/ml to about 1×10¹⁴ DRP/ml. In some embodiments, the concentration of the vector in the second fluid is about 1×10¹¹ DRP/ml. In some embodiments, the first or the second fluid further comprises a therapeutic agent. In some embodiments, the therapeutic agent is a neurotrophic factor, an anti-angiogenic factor, an anti-angiogenic polynucleotide, an anti-angiogenic morpholino, or an anti-angiogenic antibody or antigen-binding fragment thereof. In some embodiments, the first fluid is saline. In some embodiments, the fine-bore cannula is 35-41 gauge. In some embodiments, the fine-bore cannula is 40 or 41 gauge. In some embodiments, the fine-bore cannula is 41-gauge. In some embodiments, the vector is an adeno-associated virus (AAV) vector. In some embodiments, the vector is a recombinant adeno-associated virus (AAV) vector. In some embodiments, the recombinant AAV vector is an AAV2, AAV4, AAV5, or AAV8 vector. In some embodiments, the recombinant AAV vector is a pseudotyped AAV vector or chimeric AAV vector. In some embodiments, the recombinant AAV vector comprises a mixture of AAV serotypes, pseudotypes, or chimeric vectors. In some embodiments, the vector is selected from the group consisting of adenoviral, HSV, and lentiviral vectors. In some embodiments, the vector is a lentiviral vector. In some embodiments, the lentiviral vector is selected from the group consisting of HIV-1, HIV-2, SIV, FIV and EIAV. In some embodiments, the polynucleotide is selected to replace a mutated gene known to cause retinal disease. In some embodiments, the polynucleotide comprises a sequence encoding a polypeptide selected from the group consisting of: Prph2, RPE65, MERTK, RPGR, RP2, RPGRIP, CNGA3, CNGB3, and GNAT2. In some embodiments, the polynucleotide is RPE65. In some embodiments, the polynucleotide is hRPE65. In some embodiments, the polynucleotide encodes the polypeptide RPE65. In some embodiments, the polynucleotide encodes the polypeptide hRPE65. In some embodiments, the polynucleotide comprises a sequence encoding a polypeptide selected from the group consisting of a neurotrophic factor, an anti-apoptotic factor, an anti-angiogenic factor, and an anti-inflammatory factor. In some embodiments, the polynucleotide comprises a sequence encoding a polypeptide selected from the group consisting of GDNF, CNTF, FGF2, PEDF, EPO, BCL2, BCL-X, NFκB, Endostatin, Angiostatin, sFlt, IL10, IL1-ra, TGFβ, and IL4. In some embodiments, the polynucleotide comprises a sequence encoding a therapeutic RNA. In some embodiments, the polynucleotide is under the control of a promoter sequence, and the promoter sequence is hRPE65 promoter (hRPEp). In some embodiments, the vector is AAV2/2-hRPE65p-hRPE65 (SEQ ID NO:1). In some embodiments, the vector is useful for transducing retinal pigment epithelial cells. In some embodiments, the vector is useful for transducing photoreceptor cells.

In another aspect of the invention is a kit comprising a system as described herein, and instructions for use. In some embodiments, the instructions for use comprise instructions for performing the method according to any one of the embodiments described herein. In some embodiments, the instructions for use comprise instructions for performing a method for treating an ocular disorder according to any one of the embodiments described herein, the method comprising administering to the subretinal space of the central retina in an eye of a human in need thereof an effective amount of a vector; wherein the vector is useful for treatment of the ocular disorder when administered to the subretinal space of the central retina of the eye.

### BRIEF DESCRIPTION OF THE DRAWINGS

FIG. 1 is a schematic diagram of the eye showing its major structures and various routes of delivery.

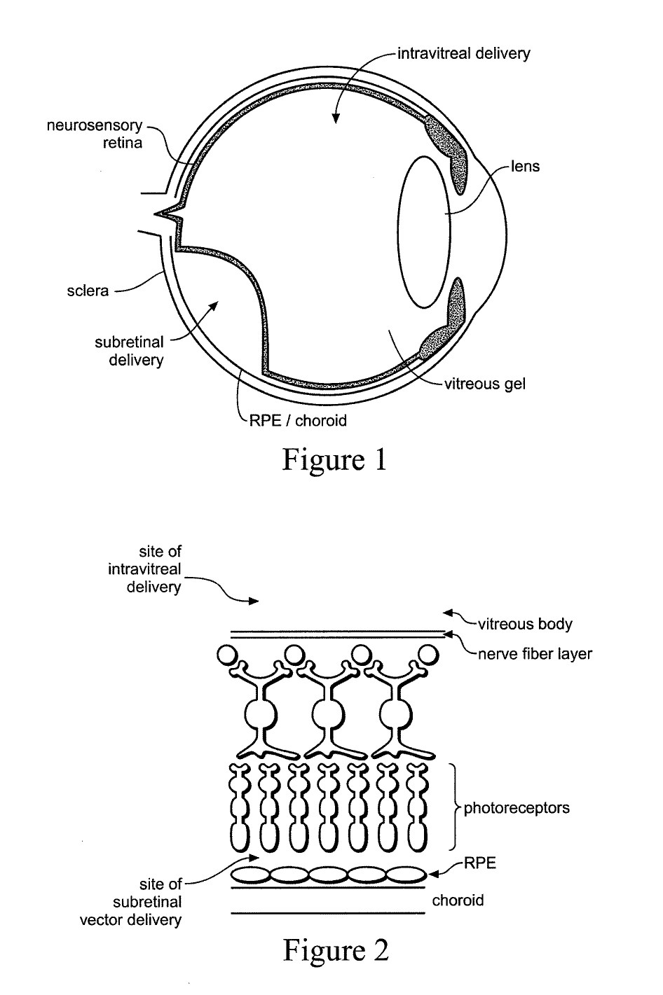

FIG. 2 is a schematic diagram of the retina showing the major cell layers and site for subretinal delivery.

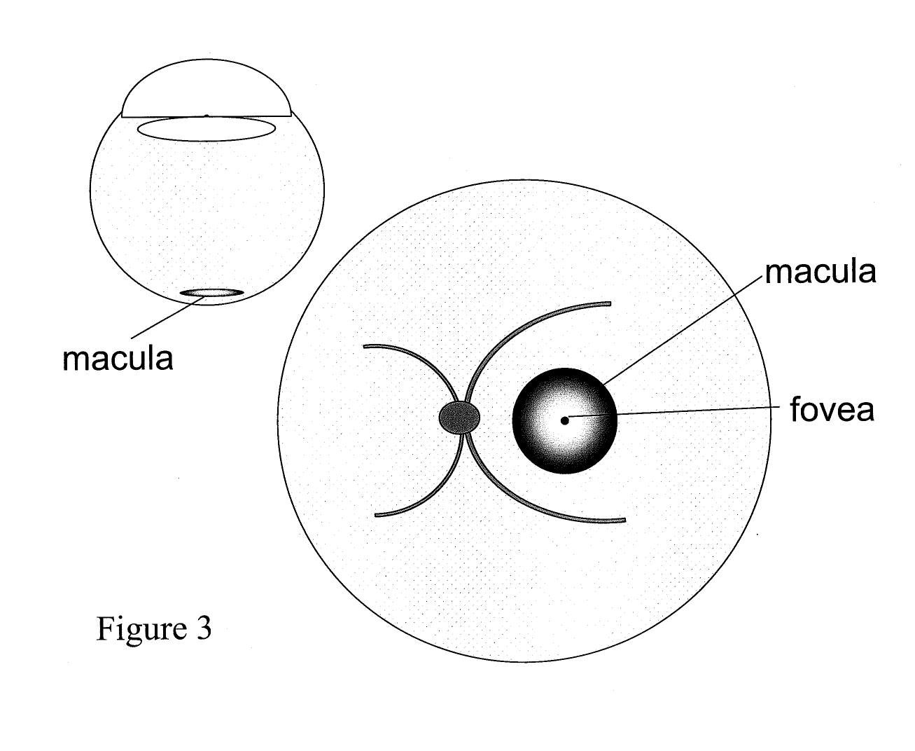

FIG. 3 is a schematic diagram of the eye showing the location of the macula and fovea.

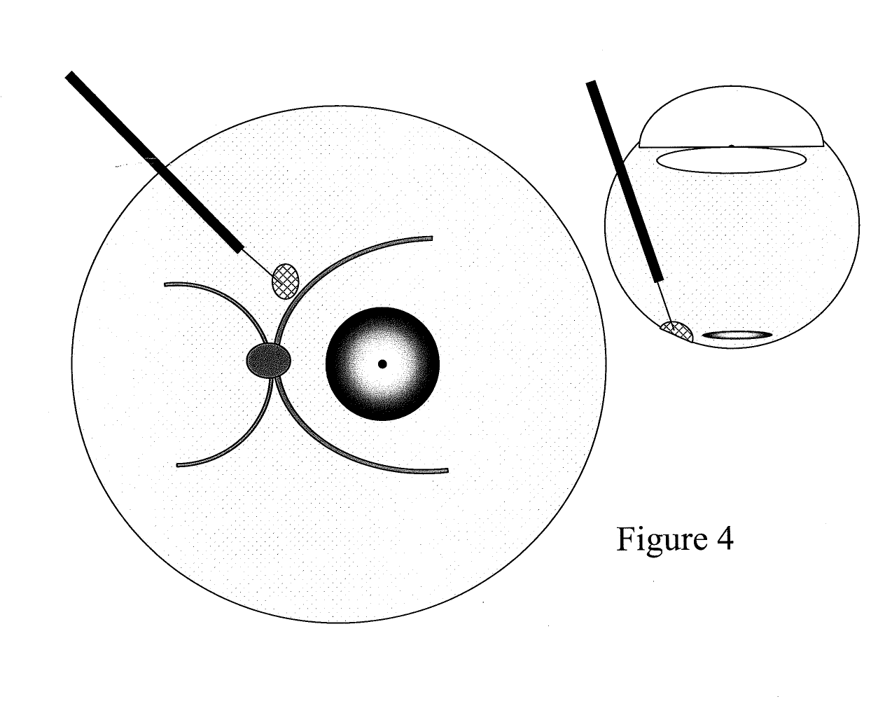

FIG. 4 is a schematic diagram of the eye showing cannular injection of a vector suspension under the retina to create a bleb (blister).

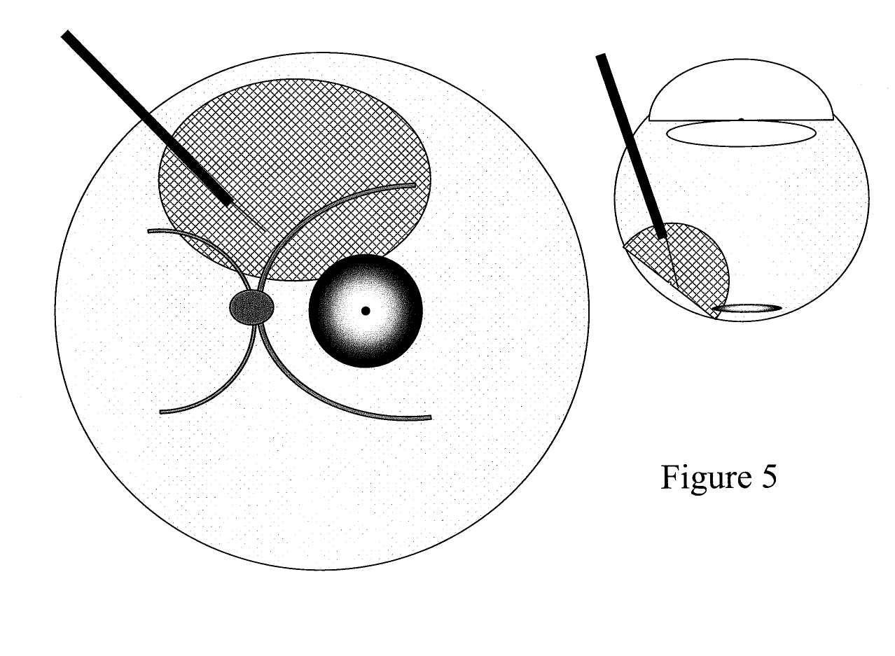

FIG. 5 is a schematic diagram of the eye showing that further injection of a vector suspension causes the bleb to enlarge.

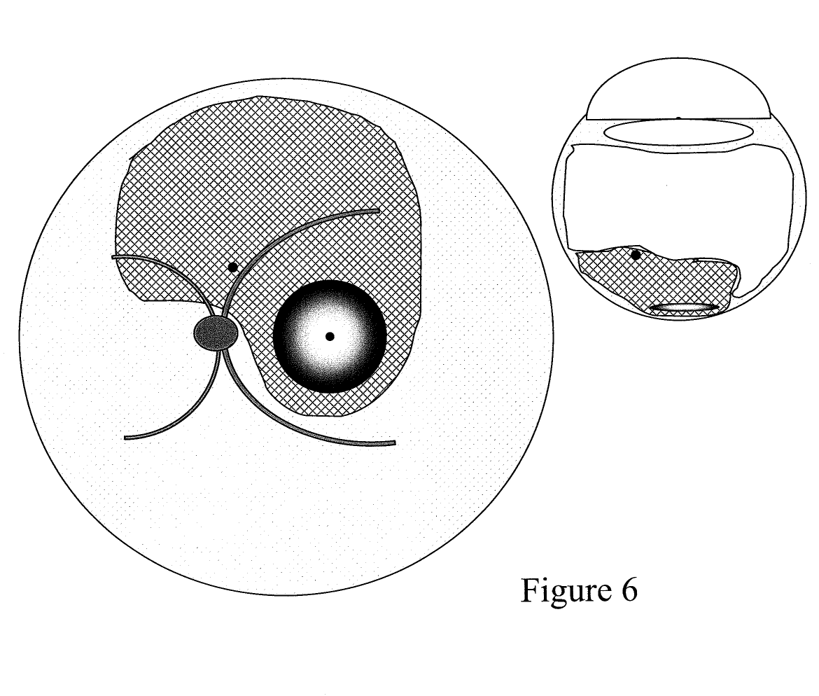

FIG. 6 is a schematic diagram of the eye showing that fluid in the vitreous cavity (in front of the retina) is replaced by air, forcing the vector suspension under the retina to extend fully under the central retina.

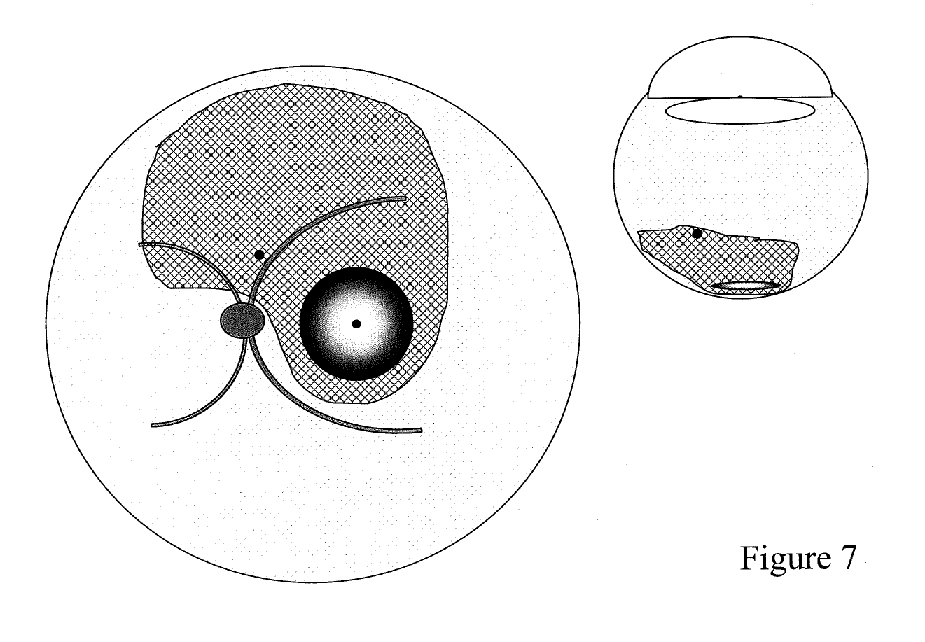

FIG. 7 is a schematic diagram of the eye showing the air in the vitreous cavity replaced by fluid, and the vector suspension remaining under the central retina.

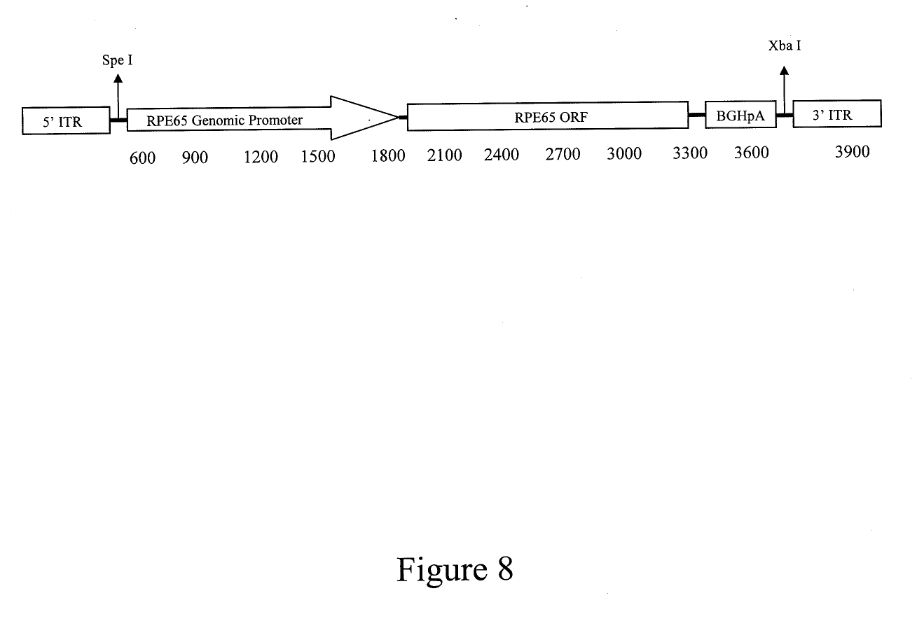

FIG. 8 shows the DNA of recombinant AAV2 AAV2/2.hRPE65P.hRPE65 Ad/AAV hybrid. The AAV2/2.hRPE65P.hRPE65 Ad/AAV hybrid DNA (SEQ ID NO:1) contains the following components: (1) AAV serotype 2-based Inverted Terminal Repeats (“ITR”) at its 3′ and 5′ ends, flanking the RPEp-RPE65-BGH polyA expression cassette. The expression cassette contains the RPE genomic promoter driving transcription of the RPE65 cDNA and a boving growth hormone (“BGH”) polyadenylation (“pA”) signal [BGHpA (GenBank Accession No. M57764)].

FIG. 9 shows the construction of Plasmid pAD3.1-RPE65. Briefly, plasmid 10/65phuRPE65 was digested with SpeI-XbaI DNA. An SpeI-XbaI fragment containing the human RPE65 promoter and the human RPE65 gene was gel-purified, and ligated into the SpeI-XbaI sites of plasmid pSH420-Delta to create plasmid pSh-Delta-huRPE65. Plasmid pSh-Delta-huRPE65 was linearized with PmeI. E. coli BJ5183 cells were electro-transformed with linear pSh-Delta-huRPE65 and pADEasy 3.1 to produce the final plasmid pAD3.1-RPE65.

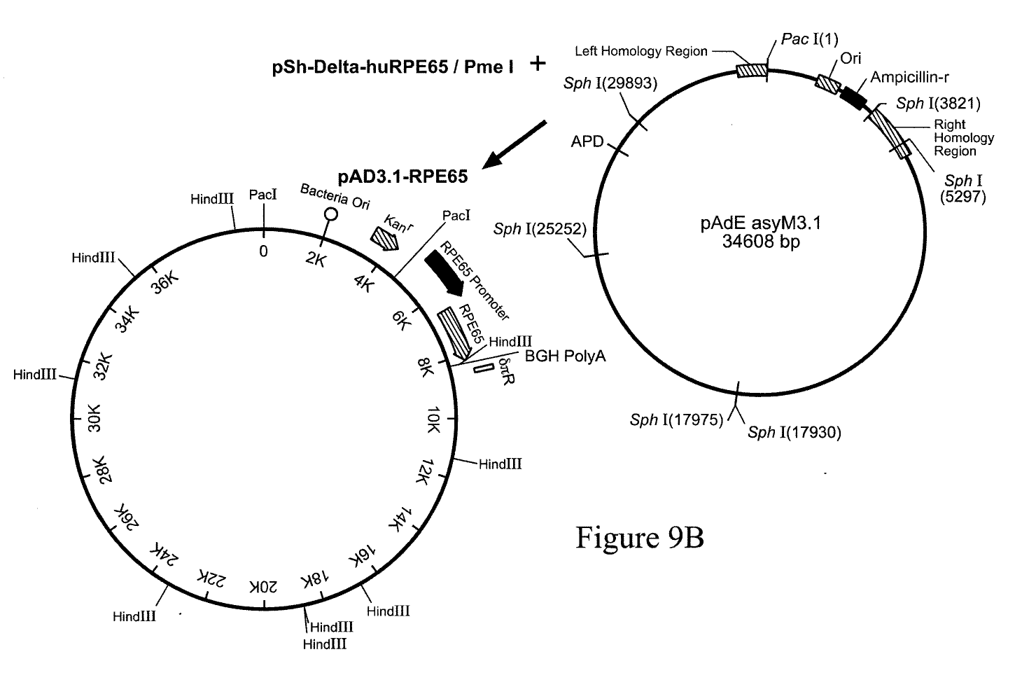

FIG. 10 show the fundus appearance of study eyes before and after vector administration.

FIG. 11 shows optical coherence tomography (OCT) images of the maculae in study eyes before and after vector administration.

FIG. 12A shows the assessment of visual function of control and study eyes by microperimetry for subject 1.

FIG. 12B shows the assessment of visual function of control and study eyes by microperimetry for subject 2.

FIG. 12C shows the assessment of visual function of control and study eyes by microperimetry for subject 3.

FIG. 13 shows the assessment of visual function by dark-adapted perimetry for subject numbers 1-3.

FIG. 14 is a schematic of the test for assessment of visual mobility.

FIG. 15A shows the assessment of visual mobility at 4 lux for subject nos. 1 and 2.

FIG. 15B shows the assessment of visual mobility at 240 lux for subject nos. 1 and 2.

FIG. 15C shows the assessment of visual mobility at 4 and 240 lux for subject no. 3.

### DETAILED DESCRIPTION OF THE INVENTION

Herein are described methods and systems for the safe and effective delivery of vectors to the macular and fovea subretina. These methods permit safe detachment of the macula and fovea by injection of the vectors subretinally outside the central retina and subsequently maneuvering the retinal detachment to the central retinal area. In particular, these methods permit the safe and effective transduction of RPE and/or photoreceptor cells of the macula and/or fovea. These methods may be used in the treatment of ocular disorders.

Without wishing to be bound by theory, the inventors have discovered a method and system for subretinal delivery of vectors encoding polynucleotides for treatment of ocular disorders, in which the method comprises creating a fluid bleb within the subretinal space outside the regions of the central retina, wherein the fluid bleb has sufficient size and volume that it causes a detachment of the retina and can be repositioned to the central retina by dependency and/or fluid-air exchange along the surface of the retina. By using such method, the cells of the macula and/or fovea are transduced in a safe and effective manner.

### DEFINITIONS

The term “central retina” as used herein refers to the outer macula and/or inner macula and/or the fovea.

The term “central retina cell types” as used herein refers to cell types of the central retina, such as, for example, RPE and photoreceptor cells.

The term “macula” refers to a region of the central retina in primates that contains a higher relative concentration of photoreceptor cells, specifically rods and cones, compared to the peripheral retina.

The term “outer macula” as used herein may also be referred to as the “peripheral macula”.

The term “inner macula” as used herein may also be referred to as the “central macula”.

The term “fovea” refers to a small region in the central retina of primates of approximately equal to or less than 0.5 mm in diameter that contains a higher relative concentration of photoreceptor cells, specifically cones, when compared to the peripheral retina and the macula.

The term “subretinal space” as used herein refers to the location in the retina between the photoreceptor cells and the retinal pigment epithelium cells. The subretinal space may be a potential space, such as prior to any subretinal injection of fluid. The subretinal space may also contain a fluid that is injected into the potential space. In this case, the fluid is “in contact with the subretinal space.” Cells that are “in contact with the subretinal space” include the cells that border the subretinal space, such as RPE and photoreceptor cells.

The term “vitrectomy space” as used herein refers to the volume space left in the vitreous cavity by removal of vitreous gel during a vitrectomy.

The term “bleb” as used herein refers to a fluid space within the subretinal space of an eye. A bleb of the invention may be created by a single injection of fluid into a single space, by multiple injections of one or more fluids into the same space, or by multiple injections into multiple spaces, which when repositioned create a total fluid space useful for achieving a therapeutic effect over the desired portion of the subretinal space.

The term “polypeptide” is used herein to refer to polymers of amino acids of any length. The terms also encompass an amino acid polymer that has been modified in vivo; for example, by disulfide bond formation, glycosylation, and/or lipidation.

The term “therapeutic polypeptide” is used herein to refer to a polypeptide useful for treatment of an ocular disorder.

The term “polynucleotide” is used herein to refer to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

The term “therapeutic RNA” is used herein to refer to a ribonucleotide that is useful for treatment of an ocular disorder. As used herein, the therapeutic RNA is produced when the ribonucleotide is transcribed from the polynucleotide delivered by the vectors as described herein. Therapeutic RNA include, but are not limited to, RNAi, ribozymes, small inhibitory RNA (siRNA), and micro RNA (miRNA).

A “vector” as used herein refers to a viral or plasmid genome comprising a polynucleotide sequence, typically a sequence of interest for the genetic transformation of a cell. In some embodiments, a vector may be a viral vector. In some embodiments, the vector is not a viral vector.

A “viral vector” as used herein refers to an encapsidated vector.

“AAV” is an abbreviation for adeno-associated virus, and may be used to refer to the virus itself or derivatives thereof. The term covers all subtypes, serotypes, pseudotypes, chimeric and both naturally occurring and recombinant forms, except where required otherwise. Generally AAVs of serotypes 1-18 are known in the art.

An “AAV viral vector” as used herein refers to an AAV vector comprising a polynucleotide sequence, typically a sequence of interest for the genetic transformation of a cell. The AAV vector may be derived from the genome of any AAV serotype with the capsid of the viral vector of the same serotype or the viral vector may be psuedotyped with capsid proteins of a different serotype or contain modifications, deletion or insertion of non-AAV or other serotype polypeptides within the capsid.

An “rAAV vector” as used herein refers to an AAV vector comprising a polynucleotide sequence not of AAV origin (i.e., a polynucleotide heterologous to AAV), typically a sequence of interest for the genetic transformation of a cell. The heterologous polynucleotide is flanked by at least one, preferably two, AAV inverted terminal repeat sequences (ITRs). As described herein, an rAAV vector can be in any of a number of forms, including, but not limited to, plasmids, linear artificial chromosomes, complexed with lipids, encapsulated within liposomes and, most preferably, encapsidated in a viral particle, particularly an AAV.

An “rAAV virus” or “rAAV viral particle” refers to a viral particle composed of at least one AAV capsid protein (preferably by all of the capsid proteins of a wild-type AAV) and an encapsidated rAAV.

A “gene” refers to a polynucleotide containing at least one open reading frame that is capable of encoding a particular RNA or protein after being transcribed or transcribed and translated.

“Recombinant” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A cell is said to be “stably” altered, transduced, or transformed with a genetic sequence if the sequence is available to perform its function during extended culture of the cell in vitro or in vivo. In some examples, such a cell is “inheritably” altered in that a genetic alteration is introduced which is also inheritable by progeny of the altered cell.

A “host cell” includes an individual cell or cell culture which can be or has been a recipient for vector(s). Host cells include progeny of a single host cell, and the progeny may not necessarily be completely identical (in morphology or in genomic or total DNA complement) to the original parent cell due to natural, accidental, or deliberate mutation. A host cell includes cells transfected in vivo with a polynucleotide(s) of this invention.

An “effective amount” is an amount sufficient to effect or achieve a beneficial or desired clinical result. An effective amount can be administered in one or more administrations. For purposes of this invention, an “effective amount” is an amount that achieves any of the following: an alleviation of symptoms, diminishment of extent of disease, preventing spread of disease, or improvement, palliation, amelioration, stabilization (i.e. not worsening), reversal, remission (whether partial or total), or slowing or delay in the progression of one or more signs or symptoms of the disease state. For example, in the case of ocular disorders that negatively affect a subject's visual function, a beneficial clinical result may be measured by, for example, the subject's subjective quality of vision or improved central vision function (e.g. an improvement in the subject's ability to read fluently and recognize faces), the subject's visual mobility (e.g. a decrease in time needed to navigate a maze), visual acuity (e.g. an improvement in the subject's LogMAR score), microperimetry (e.g. an improvement in the subject's dB score), dark-adapted perimetry (e.g. an improvement in the subject's dB score), fine matrix mapping (e.g. an improvement in the subject's dB score), Goldmann perimetry (e.g. a reduced size of scotomatous area (i.e. areas of blindness) and improvement of the ability to resolve smaller targets), flicker sensitivities (e.g. an improvement in Hertz), autofluorescence, and electrophysiology measurements (e.g. improvement in ERG).

Visual mobility measures navigational performance under strictly controlled conditions.

VA (visual acuity) is a standardized method of assessing vision (reading letters on a board decreasing size), quantified in LogMAR scale. Zero is standard vision, higher numbers is below standard vision. The same scale is used for low light VA, and reading tests (read acuity, and Max read rate). Contrast sensitivity (Pelli-Robson CS) is a standardized method for assessing vision (reading letters on a board in increasingly lighter grey) quantified in LogMAR scale.

Microperimetry is a standardized method of assessing vision measured in dB (decibel), and measures sensitivity of the retina at precise locations by compensating for eye movements. Big improvements can be assessed by an ability to resolve smaller targets (i.e. the subject sees smaller beams of light, rather than the brightness of the light).

Dark-adapted perimetry (or scotopic or Humphrey perimetry) measures retinal sensitivity by projecting light into the subject's visual field as the subject is viewing a screen in the dark.

Fine matrix mapping is standardized, measured in dB.

Goldmann perimetry is (semi)qualitative/subjective. Improvements are seen as reduced size of scotomatous area (i.e. areas of blindness) and ability to resolve smaller targets.

Flicker sensitivities are standardized, measured in Hz (Hertz).

Autofluorescence (AF) is a subjective assessment. Improvement differs per disease. In some diseases, subjects have no AF and improvement would be an increase; in other diseases AF might be high or inconsistent in subjects and improvement would be a decrease or a more even AF.

All electrophysiology (VEPs, ERGs, ON-OFF responses) is standardized and measured in μV (microvolt). Electrophysiology measurements include, for example, VEP (pattern reversal), VEP (flash), Pattern ERG, ERG (rod specific), Bright flash ERG, 30 Hz flicker, Photopic single flash ERG, Photopic ON and OFF responses, S-cone ERG, Multifocal ERG.

A method of the invention “does not significantly adversely affect central retinal function or central retinal structure” when following delivery of vector there are no significant permanent or nonresolvable adverse changes as measured by retinal assessment methods including, but not limited to: fundus examination, visual acuity, contrast sensitivity, reading speed assessment, Goldman perimetry, microperimetry, dark-adapted perimetry, fine matrix mapping, rod and cone flicker sensitivities, assessment of visual mobility, autofluorescence, optical coherence tomography, flash electroretinography, pattern electroretinography, electro-oculography and multifocal electroretinography. For example, adverse changes as measured by fundus examination include: the presence of immune cells in the AC (anterior chamber, i.e. front of the eye) or vitreous; any opacities occurring in the media (e.g. cataracts in the lens); adverse retinal morphology resulting from the procedure, such as in how the detachment is resolved (e.g. folds, lesions (holes), inflammation, persisting detachment, RPE hypertrophy (migration of RPE cells into the retina), RPE atrophy (holes in the RPE)). A clinician of skill in the art to which this invention belongs would be able to distinguish an insignificant adverse event from a significant adverse event. For example, a permanent change adversely affecting vision would be a significant adverse event; e.g. lesions and large folds. RPE damage (both hypertrophy and atrophy) in the macula will affect vision in the longer term. In contrast, temporary changes (e.g. small folds that resolve) or treatable changes (e.g. treatable inflammation) are insignificant adverse events. Significant adverse events also include an adverse event of Grade II or above, as described in the trial protocol in Example 2 below.

As used herein, “treatment” or “treating” is an approach for obtaining beneficial or desired clinical results, as described herein. For purposes of this invention, a beneficial or desired clinical result includes, but is not limited to, an alleviation of symptoms, diminishment of extent of disease, preventing spread of disease, or improvement, palliation, amelioration, stabilization (i.e. not worsening), reversal, remission (whether partial or total), or slowing or delay in the progression of one or more signs or symptoms of the disease state. Beneficial or desired clinical results include, for example, an improvement and/or stabilization and/or delay in the progression of one or more signs or symptoms of the disease state, whether evaluated by objective or subjective tests.

A method of the invention is “effective in treating the human's visual function” when it achieves any more or more of the following: improvement, palliation, amelioration, stabilization, reversal, remission, or slowing or delay in the progression of one or more signs or symptoms of the human's visual function. Visual function may be assessed by the objective and subjective tests as described herein, for example, by one or more of: the subject's subjective quality of vision or improved central vision function, the subject's visual mobility, visual acuity, microperimetry, dark-adapted perimetry, fine matrix mapping, Goldmann perimetry, flicker sensitivities, autofluorescence, and electrophysiology measurements.

“Palliating” a disease means that the extent and/or undesirable clinical manifestations of a disease state are lessened and/or time course of the progression is slowed or lengthened, as compared to not administering the vectors of the present invention.

The term “DRP” refers to DNase-resistant particles.

As used in this specification and the appended claims, the singular forms “a,” “an” and “the” include plural referents unless the context clearly indicates otherwise.

Unless defined otherwise, all technical and scientific terms used herein have the same meaning as is commonly understood by one of ordinary skill in the art to which this invention belongs.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in their respective testing measurements.

### General Techniques

The practice of the present invention will employ, unless otherwise indicated, conventional techniques of molecular biology, virology, animal cell culture and biochemistry which are within the skill of the art. Such techniques are explained fully in the literature. See, for example, “Molecular Cloning: A Laboratory Manual”, Second Edition (Sambrook, Fritsch & Maniatis, 1989); “Animal Cell Culture” (R. I. Freshney, ed., 1987); “Gene Transfer Vectors for Mammalian Cells” (J. M. Miller & M. P. Calos, eds., 1987); “Current Protocols in Molecular Biology” (F. M. Ausubel et al., eds., 1987); “Current Protocols in Protein Science” (John E Coligan, et al. eds. Wiley and Sons, 1995); and “Protein Purification: Principles and Practice” (Robert K. Scopes, Springer-Verlag, 1994).

Vectors for Delivery of Polynucleotides

This invention provides methods for the safe and effective administration of vectors (e.g. AAV or lentiviral vectors) to macular and/or fovea subretinal cells of a human. The vectors may comprise, for example, a polynucleotide encoding a polypeptide (e.g. a therapeutic polypeptide) or therapeutic RNA sequence.

In some embodiments, the vector is a recombinant AAV vector (rAAV). A rAAV vector of this invention comprises a heterologous (i.e. non-AAV) polynucleotide of interest in place of the AAV rep and/or cap genes that normally make up the bulk of the AAV genome. As in the wild-type AAV genome, however, the heterologous polynucleotide is preferably flanked by at least one, more preferably two, AAV inverted terminal repeats (ITRs). Variations in which a rAAV construct is flanked by only a single (typically modified) ITR have been described in the art and can be employed in connection with the present invention.

The rAAV vectors may be prepared using standard methods in the art. Adeno-associated viruses of any serotype are suitable, since the various serotypes are functionally and structurally related, even at the genetic level (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R MLinden, C R Parrish, Eds.) p5-14, Hudder Arnold, London, UK (2006); and D E Bowles, J E Rabinowitz, R J Samulski “The Genus Dependovirus” (J R Kerr, S F Cotmore. M E Bloom, R M Linden, C R Parrish, Eds.) p15-23, Hudder Arnold, London, UK (2006). Methods for purifying for vectors may be found in, for example, U.S. Pat. Nos. 6,566,118, 6,989,264, and 6995006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety.

Other vectors may be used, including lentiviral, HSV, and adenoviral vectors. Lentiviruses include, but are not limited to, HIV-1, HIV-2, SIV, FIV and EIAV. Lentiviruses may be pseudotyped with the envelope proteins of other viruses, including, but not limited to VSV, rabies, Mo-MLV, baculovirus and Ebola. Such vectors may be prepared using standard methods in the art. In some embodiments, the vector is a lentiviral vector. In some embodiments, the vector is selected from the group consisting of HIV-1, HIV-2, SIV, FIV and EIAV.

In some embodiments, the vector(s) for use in the methods of the invention are encapsidated into a virus particle (e.g. an rAAV virus particle). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

For any of the methods described herein, it is understood that one or more vectors may be administered to the eye. If more than one vector is used, it is understood that they may be administered at the same or at different times to the eye.

Polynucleotides Encoding Polypeptides and Therapeutic RNA

The vector may comprise a polynucleotide encoding a polypeptide (e.g. a therapeutic or diagnostic polypeptide). Polynucleotides which encode therapeutic or diagnostic polypeptides can be generated using methods known in the art, using standard synthesis and recombinant methods. In some embodiments, the polynucleotide encodes a therapeutic polypeptide. In some embodiments, the polynucleotide encodes a diagnostic polypeptide. Non-limiting examples of polynucleotides encoding therapeutic polypeptides include: polynucleotides for replacement of a missing or mutated gene known to cause retinal disease, for example Prph2, RPE65, MERTK, RPGR, RP2, RPGRIP, CNGA3, CNGB3, and GNAT2. Other non-limiting examples of polynucleotides encoding therapeutic polypeptides include those encoding neurotrophic factors (such as GDNF, CNTF, FGF2, PEDF, EPO), anti-apoptotic genes (such as BCL2, BCL-X, NFκB), anti-angiogenic factors (such as Endostatin, Angiostatin, sFlt), and anti-inflammatory factors (such as IL10, IL1-ra, TGFβ, IL4). In some embodiments, the encoded polypeptide is the human variant of the polypeptide. In some embodiments, the polypeptide is RPE65. In some embodiments, the polypeptide is hRPE65.

The polynucleotides of the invention may encode polypeptides that are intracellular proteins, anchored in the cell membrane, remain within the cell, or are secreted by the cell transduced with the vectors of the invention. For polypeptides secreted by the cell that receives the vector; preferably the polypeptide is soluble (i.e., not attached to the cell). For example, soluble polypeptides are devoid of a transmembrane region and are secreted from the cell. Techniques to identify and remove polynucleotide sequences which encode transmembrane domains are known in the art.

The vectors that can be administered according to the present invention also include vectors comprising a polynucleotide which encodes a RNA (e.g., RNAi, ribozymes, miRNA, siRNA) that when transcribed from the polynucleotides of the vector can treat an ocular disorder by interfering with translation or transcription of an abnormal or excess protein associated with a disease state of the invention. For example, the polynucleotides of the invention may encode for an RNA which treats a disease by highly specific elimination or reduction of mRNA encoding the abnormal and/or excess proteins. Therapeutic RNA sequences include RNAi, small inhibitory RNA (siRNA), micro RNA (miRNA), and/or ribozymes (such as hammerhead and hairpin ribozymes) that can treat diseases by highly specific elimination or reduction of mRNA encoding the abnormal and/or excess proteins, such as those occurring in various forms of inherited retinal degeneration. Non-limiting examples of ocular disorders which may be treated by therapeutic RNA sequences include, for example, autosomal dominant retinitis pigmentosa (ADRP) and diabetic retinopathy. Examples of therapeutic RNA sequences and polynucleotides encoding these sequences which may be used in the invention include those described in, for example, U.S. Pat. No. 6,225,291, the disclosure of which is herein incorporated by reference in its entirety.

### Vector Compositions

Generally, the compositions for use in the methods and systems of the invention comprise an effective amount of a vector encoding a polypeptide or therapeutic RNA, preferably in a pharmaceutically acceptable excipient. As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, and buffers. Excipients as well as formulations for parenteral and nonparenteral drug delivery are set forth in Remington's Pharmaceutical Sciences 19th Ed. Mack Publishing (1995).

Generally, these compositions are formulated for administration by subretinal injection. Accordingly, these compositions are preferably combined with pharmaceutically acceptable vehicles such as saline, Ringer's balanced salt solution (pH 7.4), and the like. Although not required, the compositions may optionally be supplied in unit dosage form suitable for administration of a precise amount.

Methods of Administering Vectors to Subretinal Macular and Fovea Cells

The macula and fovea regions of the retina are unique amongst mammals to primates. The macula is near the centre of the retina and has a diameter of approximately 1.5 mm. This area contains the highest concentration of both rod and cone photoreceptors. At the centre of the macula is the fovea, a small pit that contains the largest concentration of cone photoreceptors. The macula and fovea regions of the retina also contain underlying RPE cells. These regions of the retina are responsible for perception of fine detail (acuity) and colour. As this region is responsible for the most important part of human vision (fine vision), safe and effective targeting of the vector to the subretinal space of the macula and fovea is desired.

Briefly, the general method for delivering a vector to the subretinal space of the macula and fovea may be illustrated by the following brief outline. This example is merely meant to illustrate certain features of the method, and is in no way meant to be limiting. A more detailed description of one embodiment of the method according to the invention is described in Example 2.

Generally, the vector can be delivered in the form of a suspension injected intraocularly (subretinally) under direct observation using an operating microscope. This procedure may involve vitrectomy followed by injection of vector suspension using a fine cannula through one or more small retinotomies into the subretinal space.

Briefly, an infusion cannula can be sutured in place to maintain a normal globe volume by infusion (of e.g. saline) throughout the operation. A vitrectomy is performed using a cannula of appropriate bore size (for example 20 to 27 gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g. saline) creates space to accommodate the intraocular injection of vector, and (3) its controlled removal reduces the possibility of retinal tears and unplanned retinal detachment.

In some embodiments, the vector is directly injected into the subretinal space outside the central retina, by utilizing a cannula of the appropriate bore size (e.g. 27-45 gauge), thus creating a bleb in the subretinal space. In other embodiments, the subretinal injection of vector suspension is preceded by subretinal injection of a small volume (e.g. about 0.1 to about 0.5 ml) of an appropriate fluid (such as saline or Ringer's solution) into the subretinal space outside the central retina. This initial injection into the subretinal space establishes an initial fluid bleb within the subretinal space, causing localized retinal detachment at the location of the initial bleb. This initial fluid bleb can facilitate targeted delivery of vector suspension to the subretinal space (by defining the plane of injection prior to vector delivery), and minimize possible vector administration into the choroid and the possibility of vector injection or reflux into the vitreous cavity. In some embodiments, this initial fluid bleb can be further injected with fluids comprising one or more vector suspensions and/or one or more additional therapeutic agents by administration of these fluids directly to the initial fluid bleb with either the same or additional fine bore cannulas.

Intraocular administration of the vector suspension and/or the initial small volume of fluid can be performed using a fine bore cannula (e.g. 27-45 gauge) attached to a syringe. In some embodiments, the plunger of this syringe may be driven by a mechanised device, such as by depression of a foot pedal. The fine bore cannula is advanced through the sclerotomy, across the vitreous cavity and into the retina at a site pre-determined in each subject according to the area of retina to be targeted (but outside the central retina). Under direct visualisation the vector suspension is injected mechanically under the neurosensory retina causing a localised retinal detachment with a self-sealing non-expanding retinotomy. As noted above, the vector can be either directly injected into the subretinal space creating a bleb outside the central retina or the vector can be injected into an initial bleb outside the central retina, causing it to expand (and expanding the area of retinal detachment). In some embodiments, the injection of vector suspension is followed by injection of another fluid into the bleb.

Without wishing to be bound by theory, the rate and location of the subretinal injection(s) can result in localized shear forces that can damage the macula, fovea and/or underlying RPE cells. The subretinal injections may be performed at a rate that minimizes or avoids shear forces. In some embodiments, the vector is injected over about 15-17 minutes. In some embodiments, the vector is injected over about 17-20 minutes. In some embodiments, the vector is injected over about 20-22 minutes. In some embodiments, the vector is injected at a rate of about 35 to about 65 μl/ml. In some embodiments, the vector is injected at a rate of about 35 μl/ml. In some embodiments, the vector is injected at a rate of about 40 μl/ml. In some embodiments, the vector is injected at a rate of about 45 μl/ml. In some embodiments, the vector is injected at a rate of about 50 μl/ml. In some embodiments, the vector is injected at a rate of about 55 μl/ml. In some embodiments, the vector is injected at a rate of about 60 μl/ml. In some embodiments, the vector is injected at a rate of about 65 μl/ml. One of ordinary skill in the art would recognize that the rate and time of injection of the bleb may be directed by, for example, the volume of the vector or size of the bleb necessary to create sufficient retinal detachment to access the cells of central retina, the size of the cannula used to deliver the vector, and the ability to safely maintain the position of the canula of the invention.

One or multiple (e.g. 2, 3, or more) blebs can be created. Generally, the total volume of bleb or blebs created by the methods and systems of the invention can not exceed the fluid volume of the eye, for example about 4 ml in a typical human subject. The total volume of each individual bleb is preferably at least about 0.3 ml, and more preferably at least about 0.5 ml in order to facilitate a retinal detachment of sufficient size to expose the cell types of the central retina and create a bleb of sufficient dependency for optimal manipulation. One of ordinary skill in the art will appreciate that in creating the bleb according to the methods and systems of the invention that the appropriate intraocular pressure must be maintained in order to avoid damage to the ocular structures. The size of each individual bleb may be, for example, about 0.5 to about 1.2 ml, about 0.8 to about 1.2 ml, about 0.9 to about 1.2 ml, about 0.9 to about 1.0 ml, about 1.0 to about 2.0 ml, about 1.0 to about 3.0 ml. Thus, in one example, to inject a total of 3 ml of vector suspension, 3 blebs of about 1 ml each can be established. The total volume of all blebs in combination may be, for example, about 0.5 to about 3.0 ml, about 0.8 to about 3.0 ml, about 0.9 to about 3.0 ml, about 1.0 to about 3.0 ml, about 0.5 to about 1.5 ml, about 0.5 to about 1.2 ml, about 0.9 to about 3.0 ml, about 0.9 to about 2.0 ml, about 0.9 to about 1.0 ml.

In order to safely and efficiently transduce areas of target retina (e.g. the central retina) outside the edge of the original location of the bleb, the bleb may be manipulated to reposition the bleb to the target area for transduction. Manipulation of the bleb can occur by the dependency of the bleb that is created by the volume of the bleb, repositioning of the eye containing the bleb, repositioning of the head of the human with an eye or eyes containing one or more blebs, and/or by means of a fluid-air exchange. This is particularly relevant to the central retina since this area typically resists detachment by subretinal injection. In some embodiments fluid-air exchange is utilized to reposition the bleb; fluid from the infusion cannula is temporarily replaced by air, e.g. from blowing air onto the surface of the retina. As the volume of the air displaces vitreous cavity fluid from the surface of the retina, the fluid in the vitreous cavity may flow out of a cannula. The temporary lack of pressure from the vitreous cavity fluid causes the bleb to move and gravitate to a dependent part of the eye. By positioning the eye globe appropriately, the bleb of subretinal vector is manipulated to involve adjacent areas (e.g. the macula and/or fovea). In some cases, the mass of the bleb is sufficient to cause it to gravitate, even without use of the fluid-air exchange. Movement of the bleb to the desired location may further be facilitated by altering the position of the subject's head, so as to allow the bleb to gravitate to the desired location in the eye. Once the desired configuration of the bleb is achieved, fluid is returned to the vitreous cavity. The fluid is an appropriate fluid, e.g., fresh saline. Generally, the subretinal vector may be left in situ without retinopexy to the retinotomy and without intraocular tamponade, and the retina will spontaneously reattach within about 48 hours.

### Methods of Treatment

By safely and effectively transducing ocular cells (e.g. RPE and/or photoreceptor cells of e.g. the macula and/or fovea) with a vector comprising a therapeutic polypeptide or RNA sequence, the methods of the invention may be used to treat a human having an ocular disorder, wherein the transduced cells produce the therapeutic polypeptide or RNA sequence in an amount sufficient to treat the ocular disorder.

An effective amount of vector (in some embodiments in the form of particles) is administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. As a guide, the number of particles administered per injection is generally between about 1×10⁶ and about 1×10¹⁴ particles, preferably, between about 1×10⁷ and 1×10¹³ particles, more preferably about 1×10⁹ and 1×10¹² particles and even more preferably about 1×10¹¹ particles. The vector may be administered by one or more subretinal injections, either during the same procedure or spaced apart by days, weeks, months, or years. In some embodiments, multiple vectors may be used to treat the human.

The effectiveness of vector delivery can be monitored by several criteria as described herein. For example, after treatment in a subject using methods of the present invention, the subject may be assessed for e.g. an improvement and/or stabilization and/or delay in the progression of one or more signs or symptoms of the disease state by one or more clinical parameters including those described herein. Examples of such tests are known in the art, and include objective as well as subjective (e.g. subject reported) measures. For example, to measure the effectiveness of a treatment on a subject's visual function, one or more of the following may be evaluated: the subject's subjective quality of vision or improved central vision function (e.g. an improvement in the subject's ability to read fluently and recognize faces), the subject's visual mobility (e.g. a decrease in time needed to navigate a maze), visual acuity (e.g. an improvement in the subject's LogMAR score), microperimetry (e.g. an improvement in the subject's dB score), dark-adapted perimetry (e.g. an improvement in the subject's dB score), fine matrix mapping (e.g. an improvement in the subject's dB score), Goldmann perimetry (e.g. a reduced size of scotomatous area (i.e. areas of blindness) and improvement of the ability to resolve smaller targets), flicker sensitivities (e.g. an improvement in Hertz), autofluorescence, and electrophysiology measurements (e.g. improvement in ERG). In some embodiments, the visual function is measured by the subject's visual mobility. In some embodiments, the visual function is measured by the subject's visual acuity. In some embodiments, the visual function is measured by microperimetry. In some embodiments, the visual function is measured by dark-adapted perimetry. In some embodiments, the visual function is measured by ERG. In some embodiments, the visual function is measured by the subject's subjective quality of vision.

In some embodiments, the method does not result in any significant permanent adverse changes in the eye. In some embodiments, the method does not result in any permanent adverse changes in the eye. In some embodiments, the method does not result in any holes. In some embodiments, the method does not result in any folds. In some embodiments, the method does not result in any media opacities. In some embodiments, the method does not result in the presence of immune cells in the anterior chamber. In some embodiments, the method does not result in an adverse event of Grade I or above, as defined in Example 2 below. In some embodiments, the method does not result in an adverse event of Grade II or above, as defined in Example 2 below. In some embodiments, the method does not result in an adverse event of Grade III or above, as defined in Example 2 below. In some embodiments, the method does not result in an adverse event of Grade IV or above, as defined in Example 2 below. In some embodiments, the method does not result in an adverse event of Grade V, as defined in Example 2 below.

In the case of diseases resulting in progressive degenerative visual function, treating the subject at an early age may not only result in a slowing or halting of the progression of the disease, it may also ameliorate or prevent visual function loss due to acquired amblyopia. Amblyopia may be of two types. In studies in nonhuman primates and kittens that are kept in total darkness from birth until even a few months of age, the animals even when subsequently exposed to light are functionally irreversibly blind despite having functional signals sent by the retina. This blindness occurs because the neural connections and “education” of the cortex is developmentally is arrested from birth due to stimulus arrest. It is unknown if this function could ever be restored. In the case of diseases of retinal degeneration, normal visual cortex circuitry was initially “learned” or developmentally appropriate until the point at which the degeneration created significant dysfunction. The loss of visual stimulus in terms of signaling in the dysfunctional eye creates “aquired” or “learned” dysfunction (“acquired amblyopia”), resulting in the brain's inability to interpret signals, or to “use” that eye. It is unknown in these cases of “acquired amblyopia” whether with improved signaling from the retina as a result of gene therapy of the amblyopic eye could ever result in a gain of more normal function in addition to a slowing of the progression or a stabilization of the disease state. In some embodiments, the human treated is less than 30 years of age. In some embodiments, the human treated is less than 20 years of age. In some embodiments, the human treated is less than 18 years of age. In some embodiments, the human treated is less than 15 years of age. In some embodiments, the human treated is less than 14 years of age. In some embodiments, the human treated is less than 13 years of age. In some embodiments, the human treated is less than 12 years of age. In some embodiments, the human treated is less than 10 years of age. In some embodiments, the human treated is less than 8 years of age. In some embodiments, the human treated is less than 6 years of age.

In some ocular disorders, there is a “nurse cell” phenomena, in which improving the function of one type of cell improves the function of another. For example, transduction of the RPE of the central retina may then improve the function of the rods, and in turn, improved rod function results in improved cone function. Accordingly, treatment of one type of cell may result in improved function in another.

The selection of a particular vector and composition depend on a number of different factors, including, but not limited to, the individual human's medical history and features of the condition and the individual being treated. The assessment of such features and the design of an appropriate therapeutic regimen is ultimately the responsibility of the prescribing physician.

In some embodiments, the human to be treated has a genetic ocular disorder, but has not yet manifested clinical signs or symptoms. In some embodiments, the human to be treated has an ocular disorder. In some embodiments, the human to be treated has manifested one or more signs or symptoms of an ocular disorder.

Non-limiting examples of ocular disorders which may be treated by the systems and methods of the invention include: autosomal recessive severe early-onset retinal degeneration (Leber's Congenital Amaurosis), congenital achromatopsia, Stargardt's disease, Best's disease, Doyne's disease, cone dystrophy, retinitis pigmentosa, X-linked retinoschisis, Usher's syndrome, atrophic age related macular degeneration, neovascular AMD, diabetic maculopathy, proliferative diabetic retinopathy (PDR), cystoid macular oedema, central serous retinopathy, retinal detachment, intra-ocular inflammation, and posterior uveitis.

### Therapeutic Agents

In some embodiments, one or more additional therapeutic agents may be administered to the subretinal space and/or to another part of the eye. Non-limiting examples of the additional therapeutic agent include polypeptide neurotrophic factors (e.g. GDNF, CNTF, BDNF, FGF2, PEDF, EPO), polypeptide anti-angiogenic factors (e.g. sFlt, angiostatin, endostatin), anti-angiogenic polynucleotides (e.g., siRNA, miRNA, ribozyme), for example anti-angiogenic polynucleotides against VEGF, anti-angiogenic morpholinos, for example anti-angiogenic morpholinos against VEGF, anti-angiogenic antibodies and/or anti-body fragments (e.g. Fab fragments), for example anti-angiogenic antibodies and/or anti-body fragments against VEGF.

Systems & Kits

The vector compositions as described herein may be contained within a system designed for use in one of the methods of the invention as described herein. Generally, the system comprises a fine-bore cannula, wherein the cannula is 27 to 45 gauge, one or more syringes (e.g. 1, 2, 3, 4 or more), and one or more fluids (e.g. 1, 2, 3, 4 or more) suitable for use in the methods of the invention.

The fine bore cannula is suitable for subretinal injection of the vector suspension and/or other fluids to be injected into the subretinal space. In some embodiments, the cannula is 27 to 45 gauge. In some embodiments, the fine-bore cannula is 35-41 gauge. In some embodiments, the fine-bore cannula is 40 or 41 gauge. In some embodiments, the fine-bore cannula is 41-gauge. The cannula may be any suitable type of cannula, for example, a de-Juan® cannula or an Eagle® cannula.

The syringe may be any suitable syringe, provided it is capable of being connected to the cannula for delivery of a fluid. In some embodiments, the syringe is an Accurus® system syringe. In some embodiments, the system has one syringe. In some embodiments, the system has two syringes. In some embodiments, the system has three syringes. In some embodiments, the system has four or more syringes.

The system may further comprise an automated injection pump, which may be activated by, e.g. a foot pedal.

The fluids suitable for use in the methods of the invention include those described herein, for example, one or more fluids each comprising an effective amount of one or more vectors as described herein, one or more fluids for creating an initial bleb (e.g. saline or other appropriate fluid), and one or more fluids comprising one or more therapeutic agents.

In some embodiments, the volume of the fluid comprising an effective amount of the vector is greater than about 0.8 ml. In some embodiments, the volume of the fluid comprising an effective amount of the vector is at least about 0.9 ml. In some embodiments, the volume of the fluid comprising an effective amount of the vector is at least about 1.0 ml. In some embodiments, the volume of the fluid comprising an effective amount of the vector is at least about 1.5 ml. In some embodiments, the volume of the fluid comprising an effective amount of the vector is at least about 2.0 ml. In some embodiments, the volume of the fluid comprising an effective amount of the vector is greater than about 0.8 to about 3.0 ml. In some embodiments, the volume of the fluid comprising an effective amount of the vector is greater than about 0.8 to about 2.5 ml. In some embodiments, the volume of the fluid comprising an effective amount of the vector is greater than about 0.8 to about 2.0 ml. In some embodiments, the volume of the fluid comprising an effective amount of the vector is greater than about 0.8 to about 1.5 ml. In some embodiments, the volume of the fluid comprising an effective amount of the vector is greater than about 0.8 to about 1.0 ml. In some embodiments, the volume of the fluid comprising an effective amount of the vector is about 0.9 to about 3.0 ml. In some embodiments, the volume of the fluid comprising an effective amount of the vector is about 0.9 to about 2.5 ml. In some embodiments, the volume of the fluid comprising an effective amount of the vector is about 0.9 to about 2.0 ml. In some embodiments, the volume of the fluid comprising an effective amount of the vector is about 0.9 to about 1.5 ml. In some embodiments, the volume of the fluid comprising an effective amount of the vector is about 0.9 to about 1.0 ml. In some embodiments, the volume of the fluid comprising an effective amount of the vector is about 1.0 to about 3.0 ml. In some embodiments, the volume of the fluid comprising an effective amount of the vector is about 1.0 to about 2.0 ml.

The fluid for creating the initial bleb may be, for example, about 0.1 to about 0.5 ml.

In some embodiments, the total volume of all fluids in the system is about 0.5 to about 3.0 ml. In some embodiments, the total volume of all fluids in the system is about 0.5 to about 2.5 ml. In some embodiments, the total volume of all fluids in the system is about 0.5 to about 2.0 ml. In some embodiments, the total volume of all fluids in the system is about 0.5 to about 1.5 ml. In some embodiments, the total volume of all fluids in the system is about 0.5 to about 1.0 ml. In some embodiments, the total volume of all fluids in the system is about 0.8 to about 3.0 ml. In some embodiments, the total volume of all fluids in the system is about 0.8 to about 2.5 ml. In some embodiments, the total volume of all fluids in the system is about 0.8 to about 2.0 ml. In some embodiments, the total volume of all fluids in the system is about 0.8 to about 1.5 ml. In some embodiments, the total volume of all fluids in the system is about 0.8 to about 1.0 ml. In some embodiments, the volume of the fluid comprising an effective amount of the vector is about 0.9 to about 3.0 ml. In some embodiments, the total volume of all fluids in the system is about 0.9 to about 2.5 ml. In some embodiments, the total volume of all fluids in the system is about 0.9 to about 2.0 ml. In some embodiments, the total volume of all fluids in the system is about 0.9 to about 1.5 ml. In some embodiments, the total volume of all fluids in the system is about 0.9 to about 1.0 ml. In some embodiments, the total volume of all fluids in the system is about 1.0 to about 3.0 ml. In some embodiments, the total volume of all fluids in the system is about 1.0 to about 2.0 ml.

In some embodiments, the system comprises a single fluid (e.g. a fluid comprising an effective amount of the vector). In some embodiments, the system comprises 2 fluids. In some embodiments, the system comprises 3 fluids. In some embodiments, the system comprises 4 or more fluids.

The systems of the invention may further be packaged into kits, wherein the kits may further comprise instructions for use. In some embodiments, the instructions for use include instructions according to one of the methods described herein.

It should be understood that the examples and embodiments described herein are for illustrative purposes only and that various modifications or changes in light thereof will be suggested to persons skilled in the art and are to be included within the spirit and purview of this application and the scope of the appended claims.

### EXAMPLES

### Abbreviations

AAV, adeno associated virus; Ad, adenovirus; AF, autofluorescence; APC, antigen presenting cell; bps, basepairs; c.f.f., clinical flicker fusion; CRF, clinical research form; CS, contrast sensitivity; DAC, dark adaptation curve; dB, decibel; DNA, deoxyribonucleic acid; DRP, Dnase resistant particles; EOG, electrooculogram; ERG, electroretinogram/electroretinography; FA, fundus autofluorescence imaging; FMM, fine matrix mapping; GFP, green fluorescent protein; GMP, good manufacturing practice; GOSH, Great Ormond street hospital; HPRT, hypoxanthine-guanine thosphoribosyl transferase; ICH, institute of child health; IoO, institute of opthalmology; ITR, inverted terminal repeat; LCA, Leber's congenital amaurosis; MEH, Moorfields eye hospital; MerTK, Mer tyrosine kinase; mfERG, multifocal ERG; MHC, major histocompatibility complex; nAb, neutralising antibodies; OCT, optical coherence tomography; PBMC, peripheral blood mononuclear cell; PCR, polymerase chain reaction; PERG, pattern ERG; pfu, plaque forming units; rAAV, recombinant AAV; RCS, royal college of surgeons; RPE, retinal pigment epithelium; RPGR, retinitis pigmentosa GTPase regulator; RPGRIP, RPGR interacting protein; rRNA, ribosomal ribonucleic acid; SV40, simian virus 40 polyadenylation site; UCL, University College London; VA, visual acuity; vg, viral genomes.

### Example 1

### Production of Construct AAV2/2.hRPE65P.hRPE65

AAV2/2.hRPE65P.hRPE65 consists of a linear single strand of DNA packaged in a recombinant adeno-associated viral protein capsid of serotype 2 (rAAV2). The AAV2/2.hRPE65P.hRPE65 genome incorporates 290 nucleotides of the wild-type AAV (wtAAV) ITR (Inverted Terminal Repeats) sequences that provide in cis the packaging signal, a cDNA encoding human RPE65 driven by a human RPE65 genomic promoter, and a BGH (Bovine Growth Hormone) polyadenylation signal.

The human RPE65 promoter fragment was amplified from human genomic DNA. The human RPE65 cDNA sequence was amplified from human retinal cDNA. After sequencing, the hRPE65 promoter fragment and the hRPE65 cDNA were cloned into plasmid pD10 with the promoter upstream of the cDNA. The sequence was subsequently cloned in the AAV backbone.

rAAV viral vectors are made by any of a number of methods known in the art including transient transfection strategies as described in U.S. Pat. Nos. 6,001,650 and 6,258,595; stable cell line strategies as fully described in WO95/34670; or shuttle vector strategies including Adenoviral hybrid vectors as described in WO96/13598 using a rep-cap cell line as described in WO99/15685 for the adenoviral-AAV hybrid vector system (Ad hybrid system). rAAV vector production requires three common elements; 1) a permissive host cell for replication which includes standard host cells known in the art including 293-A, 293-S (obtained from BioReliance), VERO, and HeLa cell lines which are applicable for the three vector production systems described herein; 2) helper virus function which as utilized herein is a wild type adenovirus type 5 virus when utilized in stable cell line manufacture and Ad hybrid vector systems or a plasmid pAd Helper 4.1 expressing the E2a, E4-orf6 and VA genes of adenovirus type 5 (Ad5) when utilized in transfection production systems; and 3) a transpackaging rep-cap construct.

Ad hybrid production of the AAV2/2.hRPE65P.hRPE65 vectors is performed essentially as described in WO96/13598 using a rep-cap cell line as described in WO99/15685. Briefly the system originally developed by T. C. He et al and disclosed in U.S. Pat. No. 5,922,576 and available as AdEasy™ kit from QBIOgene (Irvine, Calif.) and Stratagene (La Jolla, Calif.), was modified to produce an improved system that is capable of more efficient and higher yield generation of recombinant Adenovirus/AAV hybrids (Ad/AAV hybrids). This approach utilizes two plasmid vector systems (a transfer or shuttle vector and an Adenovirus genome containing vector) that undergo bacterial recombination in competent E. coli yielding a recombinant Ad/AAV hybrid plasmid which was utilized to derive Ad/AAV hybrid viral stocks as described herein. The shuttle vector described in U.S. Pat. No. 5,922,576 contains the left ITR and encapsidation sequence of adenovirus, a multiple cloning site into which the AAV2 HIV nucleic acid antigen-transgene expression cassette is inserted, and map units 9.8-16.0 and 97.2-100 of the wild type Adenovirus type 5 genome (Ad5 wt). The shuttle plasmid known in the art (U.S. Pat. No. 5,922,576) was determined by sequencing to contain a truncated left ITR and encapsidation sequence. Specifically the left ITR of the Adenovirus type 5 ITR is 385 base pairs and the shuttle vector described in U.S. Pat. No. 5,922,576 contained only 341 base pairs, a truncation of 44 base pairs. Accordingly the left adenovirus shuttle vector sequence was excised (nucleotides 1-353) by restriction enzyme digest and replaced with a PCR generated amplicon containing nucleotides 1-420 of Ad5 wt. This improved shuttle vector is designated pSh420. Virus produced using only this modification of the shuttle vector resulted in a log higher titer (2.42×10⁸ compared to 2.30×10⁷ for an AAV luciferase vector) compared to the shuttle vector previously described in the art. In order to further improve the process the adenoviral vector genome plasmid of U.S. Pat. No. 5,922,576 was analyzed by comparison of the sequence to Ad5 wt sequences. The resulting analysis revealed five deletion, nine insertions, and nine mis-sense mutations in addition, one of the E3 deletions (2682 bp) caused a deletion of the L4 polyadenylation signal. We replaced map units 75-100 of Ad5 wt in the Adenoviral genome vector in a two step cloning process. First, pSh420 was electroporated into competent E. coli BJ5183 cells along with Ad5 wt DNA resulting in homologous recombination between overlapping Ad regions, yielding a new E-1 deleted Adenoviral genome vector designated pAdNSE-1. The pAdNSE-1 and Adenoviral genome vector described in U.S. Pat. No. 5,922,576 were both digested with Pacl and SpeI to remove the Ad5 wt map units 75-100. The SpeI-PacI fragment of the pAdNSE-1 was inserted into the PacI-SpeI digested backbone of the adenoviral vector described in the art. The new improved Adenoviral genome backbone vector was designated pAd-M1. In order to allow for larger AAV expression cassettes the pAd-M1 was digested with XbaI and a 1878 base pair fragment of the E3 adenoviral gene was removed. This deletion allowed for insertion of a full length AAV expression cassette while retaining the L4 polyadenylation site which was deleted in the adenoviral genome vector known in the art. The kinetics of this new virus demonstrated improved titers (viral production equal to Ad5 wt virus) but the kinetics of growth were retarded. In order to improve growth of the recombinant plaques a PCR generated sequence encoding the 11.6 kDa protein known as the adenovirus death protein was cloned into the XbaI site in the E3 region of the E3 deleted plasmid previously described. The resulting adenoviral genome plasmid designated pAdM3.1 is approximately 4587 base pairs smaller than Ad5 wt and therefore has room for a full length AAV cassette without exceeding the packaging capacity of adenovirus. The resulting improved Ad hybrid production system utilizing the improved pSh420 shuttle plasmid and pAdM3.1 adenoviral genome plasmid yielded production of infectious Ad hybrid viral particles at levels at least two logs higher than the vector system known in the art and approximating Adwt5 virus production (1.10×10⁹, 2.30×10⁷, and 2×10⁹, respectively utilizing a Luciferase vector). The techniques used to produce the Ad hybrids of the present invention are more fully described in PCT/US2005/027091.

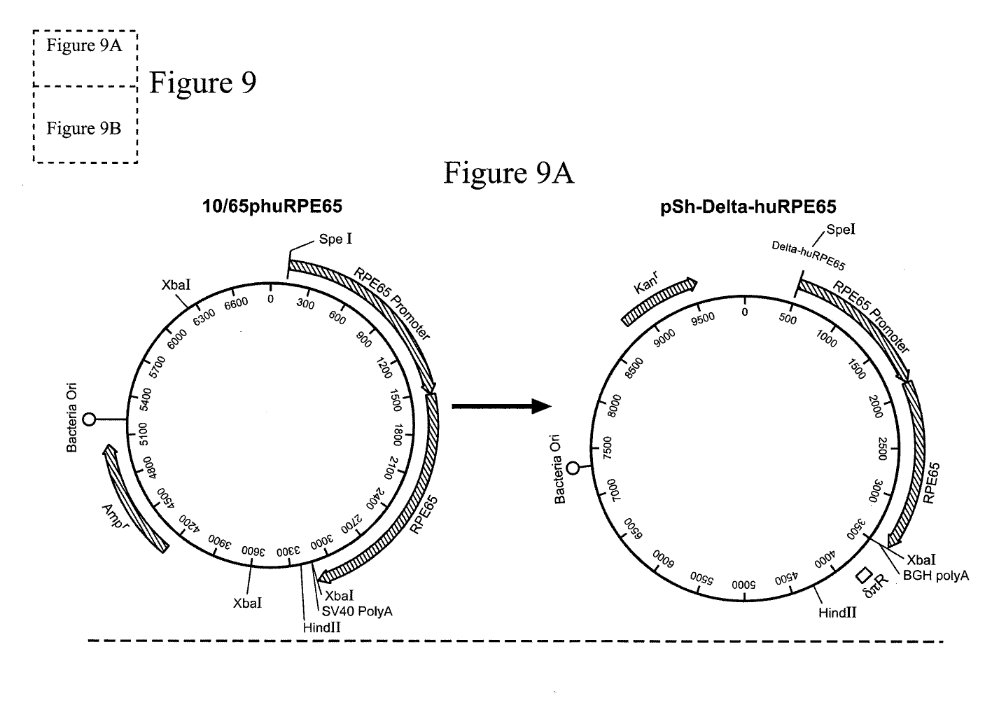

Briefly, the rAAV AAV2/2.hRPE65P.hRPE65 plasmid construct utilized to produce the rAAV vectors of the present invention was produced by ligating a 3,024 base pair SpeI and XbaI digested fragment, consisting of the human RPE65 promoter and cDNA cassette of the human RPE65 gene to was ligated to a 7.1 Kb backbone of pSh420-Delta-5′ITR to generate the intermediate plasmid pSh-Delta-huRPE65. This plasmid was linearized with enzyme PmeI and electro-transformed together with the plasmid pAdEasy M3.1 into bacteria to facilitate homologous recombination between the overlapping adenovirus DNA sequences. This recombination process generated the 30 Kb plasmid pAd3.1-RPE65. QBI-293A cells (QBIOgene, Irvine, Calif.) were then transfected with PacI linearized plasmid pAd3.1-RPE65 to produce the AAV2/2.hRPE65P.hRPE65 Ad/AAV hybrid virus. A crude stock of Ad/AAV hybrid was obtained from this transfection and plagued. The AAV2/2.hRPE65P.hRPE65 Ad/AAV hybrid plaque obtained is an E1-deleted, partial E3-deleted, recombinant Adenovirus containing the entire tgAAG76 vector genome, namely the AAV2 ITR (full 3′ and truncated 5′) flanking the RPE65 genomic promoter, the human retinal pigment epithelial (RPE65) cDNA, and the BGH polyA signal (FIG. 8). The 5′ ITR was truncated in order to reduce the potential for homologous recombination during production of the Ad/AAV hybrid viral stock. Since the 3′ ITR is intact, it will lead to regeneration of the 5′ ITR during AAV vector production. The Ad-AAV ITR AAV2/2.hRPE65P.hRPE65 Ad/AAV hybrid vectors are used to infect a stable packaging cell line expressing rep and cap along with Ad5 wt virus as a helper virus as described in WO96/13598 using a rep-cap cell line as described in WO99/15685 for the production of the rAAV. The AAV2/2.hRPE65P.hRPE65 vector was purified by opposing anion and cation chromatography using standard techniques.

Sequence of Vector Construct AAV2/2.hRPE65P.hRPE65

SEQ ID NO: 1

[the nucleotide sequence of vector construct
AAV2/2.hRPE65P.hRPE65 Ad/AAV2 hybrid]:
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTGATGTAATACGACTCACTAGTGATTCTCTC
CAAGATCCAACAAAAGTGATTATACCCCCCAAAATATGATGGTAGTATCT
TATACTACCATCATTTTATAGGCATAGGGCTCTTAGCTGCAAATAATGGA
ACTAACTCTAATAAAGCAGAACGCAAATATTGTAAATATTAGAGAGCTAA
CAATCTCTGGGATGGCTAAAGGATGGAGCTTGGAGGCTACCCAGCCAGTA
ACAATATTCCGGGCTCCACTGTTGAATGGAGACACTACAACTGCCTTGGA
TGGGCAGAGATATTATGGATGCTAAGCCCCAGGTGCTACCATTAGGACTT
CTACCACTGTCCTAACGGGTGGAGCCCATCACATGCCTATGCCCTCACTG
TAAGGAAATGAAGCTACTGTTGTATATCTTGGGAAGCACTTGGATTAATT
GTTATACAGTTTTGTTGAAGAAGACCCCTAGGGTAAGTAGCCATAACTGC
ACACTAAATTTAAAATTGTTAATGAGTTTCTCAAAAAAAATGTTAAGGTT
GTTAGCTGGTATAGTATATATCTTGCCTGTTTTCCAAGGACTTCTTTGGG
CAGTACCTTGTCTGTGCTGGCAAGCAACTGAGACTTAATGAAAGAGTATT
GGAGATATGAATGAATTGATGCTGTATACTCTCAGAGTGCCAAACATATA
CCAATGGACAAGAAGGTGAGGCAGAGAGCAGACAGGCATTAGTGACAAGC
AAAGATATGCAGAATTTCATTCTCAGCAAATCAAAAGTCCTCAACCTGGT
TGGAAGAATATTGGCACTGAATGGTATCAATAAGGTTGCTAGAGAGGGTT
AGAGGTGCACAATGTGCTTCCATAACATTTTATACTTCTCCAATCTTAGC
ACTAATCAAACATGGTTGAATACTTTGTTTACTATAACTCTTACAGAGTT
ATAAGATCTGTGAAGACAGGGACAGGGACAATACCCATCTCTGTCTGGTT
CATAGGTGGTATGTAATAGATATTTTTAAAAATAAGTGAGTTAATGAATG
AGGGTGAGAATGAAGGCACAGAGGTATTAGGGGGAGGTGGGCCCCAGAGA
ATGGTGCCAAGGTCCAGTGGGGTGACTGGGATCAGCTCAGGCCTGACGCT
GGCCACTCCCACCTAGCTCCTTTCTTTCTAATCTGTTCTCATTCTCCTTG
GGAAGGATTGAGGTCTCTGGAAAACAGCCAAACAACTGTTATGGGAACAG
CAAGCCCAAATAAAGCCAAGCATCAGGGGGATCTGAGAGCTGAAAGCAAC
TTCTGTTCCCCCTCCCTCAGCTGAAGGGGTGGGGAAGGGCTCCCAAAGCC
ATAACTCCTTTTAAGGGATTTAGAAGGCATAAAAAGGCCCCTGGCTGAGA
ACTTCCTTCTTCATTCTGCAGTTGGTAATCGAATTCATGTCTATCCAGGT
TGAGCATCCTGCTGGTGGTTACAAGAAACTGTTTGAAACTGTGGAGGAAC
TGTCCTCGCCGCTCACAGCTCATGTAACAGGCAGGATCCCCCTCTGGCTC
ACCGGCAGTCTCCTTCGATGTGGGCCAGGACTCTTTGAAGTTGGATCTGA
GCCATTTTACCACCTGTTTGATGGGCAAGCCCTCCTGCACAAGTTTGACT
TTAAAGAAGGACATGTCACATACCACAGAAGGTTCATCCGCACTGATGCT
TACGTACGGGCAATGACTGAGAAAAGGATCGTCATAACAGAATTTGGCAC
CTGTGCTTTCCCAGATCCCTGCAAGAATATATTTTCCAGGTTTTTTTCTT
ACTTTCGAGGAGTAGAGGTTACTGACAATGCCCTTGTTAATGTCTACCCA
GTGGGGGAAGATTACTACGCTTGCACAGAGACCAACTTTATTACAAAGAT
TAATCCAGAGACCTTGGAGACAATTAAGCAGGTTGATCTTTGCAACTATG
TCTCTGTCAATGGGGCCACTGCTCACCCCCACATTGAAAATGATGGAACC
GTTTACAATATTGGTAATTGCTTTGGAAAAAATTTTTCAATTGCCTACAA
CATTGTAAAGATCCCACCACTGCAAGCAGACAAGGAAGATCCAATAAGCA
AGTCAGAGATCGTTGTACAATTCCCCTGCAGTGACCGATTCAAGCCATCT
TACGTTCATAGTTTTGGTCTGACTCCCAACTATATCGTTTTTGTGGAGAC
ACCAGTCAAAATTAACCTGTTCAAGTTCCTTTCTTCATGGAGTCTTTGGG
GAGCCAACTACATGGATTGTTTTGAGTCCAATGAAACCATGGGGGTTTGG
CTTCATATTGCTGACAAAAAAAGGAAAAAGTACCTCAATAATAAATACAG
AACTTCTCCTTTCAACCTCTTCCATCACATCAACACCTATGAAGACAATG
GGTTTCTGATTGTGGATCTCTGCTGCTGGAAAGGATTTGAGTTTGTTTAT
AATTACTTATATTTAGCCAATTTACGTGAGAACTGGGAAGAAGTGAAAAA
AAATGCCAGAAAGGCTCCCCAACCTGAAGTTAGGAGATATGTACTTCCTT
TGAATATTGACAAGGCTGACACAGGCAAGAATTTAGTCACGCTCCCCAAT
ACAACTGCCACTGCAATTCTGTGCAGTGACGAGACTATCTGGCTGGAGCC
TGAAGTTCTCTTTTCAGGGCCTCGTCAAGCATTTGAGTTTCCTCAAATCA
ATTACCAGAAGTATTGTGGGAAACCTTACACATATGCGTATGGACTTGGC
TTGAATCACTTTGTTCCAGATAGGCTCTGTAAGCTGAATGTCAAAACTAA
AGAAACTTGGGTTTGGCAAGAGCCTGATTCATACCCATCAGAACCCATCT
TTGTTTCTCACCCAGATGCCTTGGAAGAAGATGATGGTGTAGTTCTGAGT
GTGGTGGTGAGCCCAGGAGCAGGACAAAAGCCTGCTTATCTCCTGATTCT
GAATGCCAAGGACTTAAGTGAAGTTGCCCGGGCTGAAGTGGAGATTAACA
TCCCTGTCACCTTTCATGGACTGTTCAAAAAATCTTGAAGCTTCGAGCGG
CCGCGACTCTAGATCATAATCAGCCATACCACATTTGTAGAGGTTTTACT
TGCTTTAAAAAACCTCCCACACCTCCCCCTGAACCTGAAACATAAAATGA
ATGCAATTGTTGTTGTTAACTTGTTTATTGCAGCTTATAATGGTTACAAA
TAAAGCAATAGCATCACAAATTTCACAAATAAAGCATTTTTTTCACTGCA
TTCTAGTTGTGGTTTGTCCAAACTCATCAATGTATCTTAAGGCCTAGGTG
AGCTCTGGTACCCTCTAGTCAAGGATCAGTGATGGAGTTGGCCACTCCCT
CTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAG

### Example 2

### Open-Dose Escalation Study of an Adeno-Associated Virus Vector (AAV2/2-hRPE65p-hRPE65) for Gene Therapy of Severe Early-Onset Retinal Degeneration

### Summary

Early-onset severe retinal dystrophy caused by mutations in RPE65 is associated with poor vision at birth. Progressive retinal degeneration typically leads to complete loss of vision in early adulthood.

As discussed in more detail in Example 3, we investigated the safety and efficacy of rAAV-mediated gene replacement therapy in 3 human subjects with this disorder, as part of an on-going clinical trial. In a phase I/II clinical trial, we delivered by subretinal injection a rAAV-2/2 vector expressing RPE65 cDNA under the control of a human RPE65 promoter (rAAV2/2.hRPE65p.hRPE65 (SEQ ID NO:1); 1×10¹¹ DRP/ml; 1000 μl) in 3 young adult human subjects. These individuals, aged 17 to 23 years, were selected on the basis of their genotype, residual visual function and degree of retinal degeneration. We examined systemic vector dissemination and immune responses following vector delivery, and performed electrophysiology, retinal imaging studies and detailed psychophysical assessments of visual function.

Subretinal administration of vector following pars-plana vitrectomy was associated with no significant intra-operative or post-operative complications. We detected no systemic dissemination of vector genome and no evidence of immune responses to rAAV vector capsid or RPE65 proteins. We found no evidence of significant adverse effects on retinal function in any of the 3 subjects.

There was no significant change in visual acuity or peripheral visual fields on Goldmann perimetry in the 3 participants. We detected no change in retinal responses by electroretinography. In the youngest subject we identified a significant improvement in visual performance consistent with improved function of rod photoreceptors resulting from expression of functional RPE65. In this subject we demonstrated an improvement in visual function by micro-perimetry and by dark-adapted perimetry. This subject also showed improvement in a subjective test of visual mobility.

The results of this study suggest that this procedure for subretinal administration of rAAV vector is safe in humans with severe retinal dystrophy and can lead to improved visual function. These findings suggest that further clinical studies are feasible and justified.

### Introduction

Efficacy and safety of subretinal administration of a recombinant adeno-associated viral vector (rAAV 2/2.hRPE65p.hRPE65 (SEQ ID NO:1)) at three different dosage levels in individuals with autosomal recessive severe early-onset retinal degeneration due to mutations in RPE65 are evaluated. Subretinal delivery of this vector results in improved visual function, and any toxicity is mild, dose-dependent and reversible.

This is the first proposal for a clinical trial of gene therapy for ocular disease in the UK. The eye is highly suited to gene therapy since delivery of vector suspension can be targeted within defined ocular compartments with minimal systemic exposure. The unique ocular immune environment is likely to confer a protective advantage against possible immune responses to capsid proteins or transgene product. The safety is further enhanced by restricting transgene expression to the target tissue by virtue of rAAV vector tropism and promoter sequence.

Retinal degeneration due to RPE65 mutations has a number of features that suggest it will be particularly amenable to a gene replacement approach. In this condition an absence of functional RPE65, an enzyme critical for cellular responses to light, results in very poor vision and leads inevitably to blindness in the third decade of life. Photoreceptor cell death occurs relatively late in the disease process and therefore the “time window” when novel therapies may be effective extends into the second decade. Delivery of the functional enzyme by gene therapy leads to measurable improvements in visual function within a short period of time.

### Design of Clinical Trial

The trial is an open label non-randomised, dose-escalation, phase I/II study involving a single subretinal administration of rAAV.hRPE65p.hRPE65 (SEQ ID NO:1) in up to 12 subjects with retinal dystrophy due to mutations in RPE65.

The study objectives are assessed in each subject for 12 months, followed by life-long follow up. The endpoint for toxicity for each subject is a Grade III adverse event, defined as loss of visual acuity by 15 or more Early treatment for diabetic retinopathy study (ETDRS) letters, or severe unresponsive intraocular inflammation. The endpoint for efficacy for each subject is defined as any improvement in visual (rod- or cone-derived) function as determined by an array of psychophysical and electrophysiological techniques, that is greater that the test-retest variation for each test.

### Subject Population in Clinical Trial

Subjects with confirmed diagnosis of severe early-onset retinal degeneration due to missense mutations in the RPE65 gene in the age range 8-30 years are enrolled. Such individuals are old enough to complete the phenotypic assessment, but still have useful residual retinal function and might be expected to benefit from intervention if it were successful. A maximum of 12 subjects are enrolled in the study.

Inclusion in the trial is limited to individuals who: (1) have severe early-onset retinal dystrophy; (2) are homozygous or compound heterozygous for a mis sense mutation(s) in RPE65; (3) are aged 8 to 30 years; (4) are able to give informed consent, with or without the guidance of their parent/guardian where appropriate. In each subject, the eye with the worse acuity is selected as the study eye, with the contralateral eye being used as a control.

Individuals are excluded who: (1) are homozygous/compound heterozygous for a null mutation in RPE65; (2) have visual acuity in the study eye better than 6/36 Snellen; (3) have contraindications for transient immune-suppression (hypertension, diabetes mellitus, tuberculosis, renal impairment, immunocompromise, osteoporosis, gastric ulceration, severe affective disorder); or (4) are pregnant or lactating women.

Individuals with mutations in RPE65 are being identified as part of an ongoing COREC approved study, based at Moorfields Eye Hospital and Great Ormond Street Hospital for Children. The aim of this study is to establish genotype-phenotype correlations in the early onset retinal dystrophies. Individuals with early onset retinal dystrophies are being identified through the genetics database based at Moorfields (which contains details of all subjects with a genetic condition affecting the retina), and electroretinography reports at Great Ormond Street as well as ongoing recruitment through clinic attendances on both sites. Over 150 subjects have been recruited to this study and have provided DNA samples for genotyping. Direct sequencing of each of the 14 exons of RPE65 has been performed in the genotyping centre based at The Institute of Opthalmology. To date, 13 subjects from 8 families ranging in age from 2-46 years have been identified. These, and any further subjects identified by the study, may be candidates for the gene therapy trial. Before enrollment in the trial, the genotypes of affected individuals are confirmed using a new blood sample in a NHS accredited diagnostic laboratory (Manchester Regional Genetic Laboratory).

As part of the ongoing approved genotype-phenotype study, subjects undergo detailed evaluation of retinal structure and function using clinical assessment, retinal imaging techniques, psychophysical and electrodiagnostic investigations. The investigations employed and protocols followed depend to some extent on the ability of the individual tested and their degree of residual retinal function. The results of these investigations are used to help to identify those subjects who are suitable subjects for inclusion in the gene therapy study.

(i) Retinal imaging. Imaging of the retina includes colour fundus photography, fundus autofluorescence imaging (FA) and optical coherence tomography (OCT). FA imaging performed with a modified scanning laser opthalmoscope allows visualisation of the RPE by taking advantage of its intrinsic fluorescence derived from its lipofuscin content. OCT imaging enables measurements of retinal thickness and provides information about the integrity of the layers of the retina.

(ii) Functional assessment. Functional assessment includes, e.g., visual acuity, contrast sensitivity, color vision, and cone flicker sensitivities. Visual acuity is measured using a LogMAR test. Contrast sensitivity is measured using the Pelli-Robson chart and colour vision using HRR plates and Mollon-Rifkind tests. Visual field testing is performed using microperimtery, dynamic perimetry (Goldmann), and photopic- and scotopic-automated static perimetry. Subjects also undergo dark-adapted perimetry using a modified Humphrey perimeter that allows rod and cone sensitivity to be measured independently. All testing follows standardized, detailed protocols, with controlled room lighting, dark-adaptation period, and a fixed sequence of test patterns. Both microperimetry and dark-adapted perimetry are fully automated so there is little opportunity for experimenter bias. Visual mobility at different illumination levels is measured by ability to navigate a simulated street scene. Flash electroretinography (ERG) and pattern ERG (PERG) are performed according to ISCEV (International Society for Clinical Electophysiology of Vision) standards to assess full field (rod and cone) and central retinal function respectively. Multifocal ERG testing is also performed where appropriate.

The first three subjects (aged 17, 18, and 23 years) to receive the IMP (AAV2/2-hRPE65p-hRPE65) were aged 16 years or older. Each had little or no vision in low light from an early age but retained some limited visual function in good lighting conditions. These subjects were selected because they retained a limited degree of residual function, despite advanced retinal degeneration, and might therefore be expected to benefit from intervention.

### Procedure for Administration of Vector

### Preoperative Procedure.

Subjects are screened to ensure there are no contra-indications for transient immune suppression, in particular, a history of hypertension, diabetes mellitus, tuberculosis, renal impairment, immunocompromise, osteoporosis, gastric ulceration or severe affective disorder. A detailed assessment of visual function and imaging is performed on both eyes preoperatively. Blood is sampled in order to assess baseline levels of circulating antibodies against AAV2 and RPE65 so that following intervention, immunological responses to vector capsids and transgene product can be determined following vector administration. Most subjects have pre-existing circulating antibodies against AAV2, but not circulating antibodies against RPE65.

For prophylaxis against potential intraocular immune responses to the IMP, subjects are prescribed a course of oral prednisolone commencing at a dose of 0.5 mg/kg one week prior to IMP administration; 1 mg/kg for the first week following administration, 0.5 mg/kg for the second week, 0.25 mg/kg for the third week and 0.125 mg/kg for the fourth week. A proton pump inhibitor is prescribed as prophylaxis against corticosteroid-induced gastric ulceration only if the subject has a history of indigestion, hiatus hernia, gastro-oesphageal reflux or is using non-steroidal anti-inflammatory drugs.

### Technique for Intraocular Administration of IMP.

The recombinant vector is delivered in the form of a suspension of viral vector particles injected intraocularly (subretinally) under direct observation using an operating microscope. This procedure involves 3-port pars plana vitrectomy followed by injection of vector suspension using a fine cannula through one or more small retinotomies (a maximum of three) into the subretinal space.

The eye and face are prepared using povidone iodine solution as per routine intraocular surgery. Following 1 minute of exposure, the excess is wiped away with sterile gauze. The face and eye are covered with an adhesive sterile plastic drape. An opening is made at the point of the palpable fissure and a wire speculum inserted to retract the upper and lower eyelids. The speculum and all intraocular instruments are sterilised according to standard Department of Health protocols. Limbal peritomies are made at two sites; one supero-nasal and the other extending along the temporal limbus. Diathermy is applied to the vessels on the cut edge to achieve haemostasis. An inferotemporal sclerostomy is made at the site of the pars plana, four millimetres posterior to the limbus. A 4 mm stainless steel intraocular infusion cannula is sutured in place to maintain a normal globe volume by infusion of saline throughout the operation. Two further sclerotomies using a 20 gauge MVR knife are made supero-temporal and supero-nasal 4 mm from the limbus. The fundus is viewed by means of a BIOM indirect viewing system or a contact lens. A 20 gauge light pipe is inserted through one of this sclerostomies and a disposable vitrectomy cutter through the other. A moderate anterior or core vitrectomy is performed before inducing a posterior vitreous detachment, as far as possible, by aspiration over the optic nerve head. Vitrectomy is completed using the disposable cutter. Clearance of sclerostomies of vitreous is performed ensuring ‘free flow’ of fluid through the ports, a standard measure to minimise vitreous incarceration and any risk of peripheral retinal breaks.

Intraocular administration of the viral vector suspension (AAV2/2-hRPE65p-hRPE65 (SEQ ID NO:1)) is performed using a de-Juan 41-gauge cannula attached to a 10 ml syringe. The plunger of this syringe is driven by a mechanised device driven by depression of a foot pedal. The 41-gauge cannula is advanced through the sclerotomy, across the vitreous cavity and into the retina at a site pre-determined in each subject according to the area of retina to be targeted. Under direct visualisation the vector suspension (1×10¹¹ vg/ml; up to a maximum dose of 3 ml) is injected mechanically under the neurosensory retina causing a localised retinal detachment with a self-sealing non-expanding retinotomy. The injection of vector suspension is preceded by injection of a small volume of Ringer's solution (0.1 to 0.5 ml) to establish the bleb, facilitating targeted delivery of vector suspension to the subretinal space and minimising possible exposure of the choroid and vitreous cavity to vector. A sample of uninjected vector suspension is retained for subsequent analysis of bioactivity.

In order to transduce areas of target retina outside the edge of the initial bleb, the bleb is manipulated by means of a fluid-air exchange. This is particularly relevant to the central retina since this area typically resists detachment by subretinal injection. Replacement of fluid in the vitreous cavity by air causes the bleb of vector under the retina to gravitate to a dependent part of the eye. By positioning the globe appropriately, the bleb of subretinal vector is manipulated to involve adjacent areas. In some cases, the mass of the bleb is sufficient to cause it to gravitate, even without use of the fluid-air exchange. Movement of the bleb to the desired location may further be facilitated by altering the position of the subject's head, so as to allow the bleb to gravitate to the desired location in the eye. Once the desired configuration of the bleb is achieved, fluid is returned to the vitreous cavity. The subretinal vector is left in situ without retinopexy to the retinotomy and without intraocular tamponade.

Following intraocular administration of vector suspension, a careful examination of the entire retinal periphery is made for any unplanned retinal breaks. Peripheral breaks are treated by cryo- or laser-retinopexy, and sterile air is injected to fill two-thirds of the intraocular volume so as to tamponade the tear without compressing the induced retinal detachment. All intraocular instruments used subsequent to vector delivery are disposable and are destroyed after a single use. Closure of all sclerotomies is completed with 7/0 vicryl solution. Standard doses of cefuroxime antibiotic and betamethasone are administered subconjunctivally as prophylaxis against postoperative infection and inflammation respectively. Atropine is instilled into the conjunctival sac to maintain pupillary mydriasis post-operatively and subtenon's marcaine for analgesia.

Following administration of vector suspension, the area of the induced retinal detachment is documented by fundus photography. The specific area of retina to be targeted in each subject is pre-determined according to the degree and distribution of retinal degeneration defined by pre-operative assessments. In the first 4 subjects vector suspension is delivered to an area amounting to no more than 30% of the total retinal area. Depending on responses in the first 4 subjects, subsequent subjects receive escalated doses of vector suspension involving larger proportions of the retinal area.

Based on preclinical studies, it is predicted that retinal blebs created by subretinal vector delivery resolve spontaneously with retinal re-attachment during the first 48 hours without the need for retinopexy or intraocular tamponade.

Surgery is performed, as is conventional for intra-ocular procedures, on a day-case basis. At least 2 hours following recovery, the operated eye is examined. Intraocular pressure of greater than 30 mmHg are managed using appropriate ocular antihypertensive therapy. The criterion for discharge is intraocular pressure of 30 mmHg or less, with or without the use ocular antihypertensive therapy as indicated. Subjects receive subsequent routine management as outsubjects, though hospital-based accommodation may be used for convenience.

### Post Operative Procedure

On the first postoperative day a full clinical examination is performed. In particular, visual acuity, intraocular pressure, the degree of postoperative intraocular inflammation and the area of any residual retinal bleb are documented. Fundus photography is performed.

A standard post-vitrectomy treatment regimen of topical antibiotic (chloramphenicol 0.5% qds for 7 days), steroid (dexamethasone 0.1% qds for 4 weeks) and mydriatic (atropine 1% bd for 7 days) is commenced to minimise inflammation and protect against infection postoperatively.

Subjects are maintained on oral prednisolone for 4 weeks following administration of vector suspension as described above (pre operative procedure). The possible development of steroid-induced adverse effects are monitored regularly. In particular, blood pressure, blood glucose, renal function and liver function are measured.

Female subjects of childbearing potential and male subjects with partners of childbearing potential are advised to use a barrier method of contraception for 12 months after enrollment.

### Assessment of Safety and Efficacy

### Effect of Agent Administration on the Eye and Visual Function

The safety and efficacy of the IMP is evaluated using a comprehensive array of clinical assessments and investigations at baseline and at defined time-points following intraocular administration.

The function of the treated retina using psychophysical assessments or rod and cone mediated visual function are assessed. This includes photopic testing using the Goldman perimeter, Humphrey static perimetry and microperimetry. Rod and cone thresholds are measured using a modified Humphrey perimeter (dark adapted perimetry). In appropriate cases detailed evaluation of the border area between treated and untreated retina by fine matrix mapping is performed. Retinal function is also assessed using full field flash electroretinography and pattern ERG. Where possible, regional responses from the retina using multifocal ERG are determined and retinal function between treated and untreated areas of retina within the same eye are compared.

The exact combination and nature of the investigations performed is tailored to the abilities of individual subjects in order to maximise the yield of information in each case. Any improvement in visual function following the intervention, greater than the test-retest variability for each assessment, is considered evidence of efficacy.

The fundus appearance and the retinal thickness are documented by fundus photography and ocular coherence tomography. Fundus autofluorescence is a feature of the retina that reflects from accumulation of lipofuscin as a result of phototransduction and the turnover of outer segment discs. In subjects with RPE65 mutations, the failure of phototransduction results in minimal accumulation of lipofuscin and characteristically low levels of fundus autofluorescence. Facilitation of phototransduction by delivery of functional RPE65 results in the accumulation of lipofuscin and an increase in autofluorescence that occurs relatively rapidly in successfully treated retinal pigment epithelium.

Clinical assessments include: (1) best corrected visual acuity (BCVA); (2) contrast sensitivity (CS); (3) reading speed assessment; (4) Goldman kinetic perimetry; (5) microperimetry; (6) Humphrey static light-adapted and dark-adapted perimetry; (7) fine matrix mapping (FMM); (8) rod and cone flicker sensitivities; (9) visual mobility; (10) fundus photography; (11) autofluorescence (AF); (12) optical coherence tomography (OCT); (13) flash eletroretinography (ERG); (14) pattern electroretinography (PERG); (15) electro-oculography (EOG); and (16) multifocal electroretinography (mfERG).

The stated time points for the schedule of visits are intended to give an accurate indication of the intervals between assessments but a degree of flexibility regarding the exact dates for assessments is maintained. Clinical assessments including measurement of visual acuity and slitlamp biomicroscopy, and fundus photography and ocular coherence tomography are performed at 1 day, 2 days, 4 days, 7 days, 2 weeks, 3 weeks, 4 weeks, 6 weeks, 8 weeks, 3 months, 4 months, 8 months and 12 months postoperatively. Where appropriate, assessments are undertaken in the Clinical Trials Unit at Moorfields Eye Hospital. In the event of any significant adverse effects, additional assessments are performed.

In addition, detailed phenotypic assessments are performed at 8 weeks, 4 months and 12 months following administration of the IMP. These assessments are scheduled over a period of up to 3 days for each time point. Assessments include contrast sensitivity, reading speed, Goldman perimetry, Humphrey static light- and dark-adapted perimetry, rod and cone flicker sensitivities, fundus autofluorescence and electrophysiology. Visual mobility is assessed at baseline and at 6 months following vector administration.

### Intraocular Inflammation

The degree of intraocular inflammation is assessed by slitlamp biomicroscopy at each time point. A temporary intraocular inflammatory response is invariable following vitrectomy surgery. This is typically evident clinically on slit lamp biomicroscopy as ‘flare’ in cells in the anterior chamber and can be of moderate (2+) intensity. The degree of intraocular inflammation reduces during the course of the first 4 weeks following the surgical procedure at which time the routine topical and systemic immunosuppression are discontinued. Prolonged or increased signs of intraocular inflammation are managed conventionally by further topical and/or systemic immunosuppression.

### Evaluation of Immune Responses

Antibody and T cell responses to AAV capsid proteins, and to RPE65 protein are investigated by ELISA and ELIspot assays at baseline and at 8 weeks, 4 months and 12 months following vector administration.

### Evaluation of Biodistribution

Systemic biodistribution of vector genomes is assessed by qPCR of tears, saliva, serum at 1 day and at 4 weeks following intraocular vector administration. Where appropriate, semen is analysed at the 4 week time-point for the presence of vector gemones.

### Long Term Follow Up

The aim of long-term follow-up is to establish safety of our approach over the lifetime of the subjects. In the first 2 years after gene transfer the subjects are reviewed at 4-monthly intervals and thereafter the follow-up visits are on an annual basis in the routine ophthalmic genetic clinic. These visits involve comprehensive clinical assessment including a detailed update of general medical conditions, detailed ocular examination including slitlamp biomicroscopy and funduscopy, and digital fundus photography.

In the UK following appropriate consent, continued surveillance of all participating subjects through a nationwide monitoring mechanism using the NHS central register and flagging system is maintained. In addition following informed consent, this system is used to follow any children born to a subject after gene therapy until the age of 16 years to study the effects of gene therapy on future generations.

### Data Analysis and Safety Monitoring Plan

### Independent Data Monitoring

An Independent Data Monitoring Committee (IDMC) is established. The IDMC consists of members with specific expertise in opthalmology and molecular genetics in addition to a subject representative. The IDMC meets to review the safety data after the first 4 subjects received the IMP at the lowest dose. This is at least 6 months after the IMP is administered to the first subject and prior to dose escalation.

### Statistical Consideration

The evaluation of efficacy is performed at 2 months, 4 months and 12 months. Data analysis is mostly descriptive in nature but data collected in a longitudinal manner is analysed if appropriate.

### Monitoring and Reporting

### Evaluation of Adverse Events

Adverse event (AE) means any untoward medical occurrence in a subject that appears to worsen between enrollment and 12 months after vector delivery, regardless of the relationship to the IMP.

Adverse Reaction (AR) means any untoward and unintended response to the IMP that is related to any dose administered in that subject.

Serious Adverse Event (SAE), Serious Adverse Reaction, or Unexpected Serious Adverse Reaction means an adverse event, adverse reaction or unexpected adverse reaction respectively that requires prolonged hospitalisation, results in persistent or significant disability or incapacity, is life threatening or consists of a congenital anomaly or birth defect.

Suspected Serious Adverse Reaction (SSAR) means a serious adverse reaction that is consistent with the information about the IMP set out in the Investigator's Brochure.

Suspected Unexpected Serious Adverse Reaction (SUSAR) means a serious adverse reaction that is not consistent with the information about the IMP set out in the Investigator's Brochure.

### Evaluation of Causality

The relationship of an adverse event with the IMP is categorized as follows: 5, unrelated; 4, unlikely to be related; 3, possibly related; 2, probably related; 1, definitely related.

### Evaluation of Severity

The grading of AEs follows standard ICH-GCP guidance. The interpretations and grading of intraocular inflammation is specific to the study yet standard for intraocular procedural studies (Standardization of Uveitis Nomenclature (SUN) Working Group Am J. Opthalmol. 2005 140 (3)).

### Minimal Adverse Events (Grade I)

Minimal adverse events are defined as: (1) significant ocular discomfort persisting more than 2 weeks and (2) Mild intraocular inflammation (1+ cells) persisting longer than 4 weeks.

### Moderate Adverse Events (Grade II)

Moderate adverse events are defined as: (1) technical complication of surgery (traumatic cataract, retinal tear or unplanned retinal detachment); (2) moderate intraocular inflammation (2+ cells) persisting for longer than 4 weeks; (3) persistently raised intraocular pressure (greater than 30 mm Hg for 3 days).

### Severe Adverse Event (Grade III)

Severe adverse events are defined as: (1) deterioration of visual acuity by 15 or more ETDRS letters or LogMAR equivalent; (2) severe unresponsive intraocular inflammation (3+ cells, choroiditis, retinitis, vasculitis)—Infective endophthalmitis.

### Very Severe Adverse Event (Grade IV)

Severe adverse events are defined as: (1) Loss of light perception; (2) Development of ocular malignancy.

Adverse events not listed in this toxicity scale are graded by the investigators as follows: I, mild; II, moderate; III, severe; IV, very severe. Any Grade III or IV adverse events are considered SAEs.

### Evaluation of Expectedness

The expectedness of a SAE/SAR is determined with reference to the Investigator's Brochure. The event is considered unexpected if it adds significant information on the specificity or severity of an expected event.

### Criteria for Dose-Escalation

Up to 12 subjects receive one of 3 different doses of vector suspension; 4 subjects are administered vector at each dose. Each subject is monitored for 8 weeks following vector administration before the next enrolled subject receives the IMP.

Dose-escalation is achieved by increasing the area of retina transduced. In the first 4 subjects one third of the retina is exposed to vector at a titre of 1×10¹¹ vg/ml using a maximum volume of 1 ml (a safe dose predicted by pre-clinical studies). The dose is subsequently increased to expose up to two-thirds and then almost the entire retina using a maximum volume of 3 ml. Dose-escalation takes place only after the safety and tolerability at the lower dose is carefully evaluated in 2 subjects for 3 months by clinical assessment of intraocular inflammation and visual function.

### Study Administration

### IMP Packaging, Labelling, Storage, Dispensing

The IMP is a recombinant serotype 2 adeno-associated viral vector containing a human RPE65 cDNA driven by a 1.6 kb fragment of the human RPE65 promoter and terminated by the bovine growth hormone polyadenylation site. The IMP was manufactured by Targeted Genetics Corporation, Seattle, Wash., USA in accordance with current Good Manufacturing Practice for clinical trial materials, using a B50 packaging cell line, an adenovirus/AAV hybrid shuttle vector containing the tgAAG76 vector genome and an adenovirus 5 helper virus.

The IMP is filled in a 2 ml Type 1 glass vial and capped with a 13 mm Fluortec Coated B2-40 grey butyl rubber stopper. An aluminium Flip Top Seal secures the stopped to the vial. All components of the container system are supplied by West Pharmaceutical Services Inc. Lionville, Pa., USA. The IMP is stored upright at −70+/−10 degrees C. in controlled and monitored freezers until time of shipping. The product, which is stable at −70 degrees Celsius (+/−10 degrees Celsius), is stored in one of two freezers (Wo Daikei ULTF80) at ≦−60° Celsius.

The label describes the following information: Lot No: HDV-0001; tgAAG76 @1×10″ DRP/mL; Subretinal Administration; 1 vial @ 1 mL each; up to 3 mL per eye; transport and store at ≦−60° C.

### Example 3

### Summary

3 subjects (aged 17, 18, 23) were enrolled and tested according to the clinical protocols of Example 2. Each subject had little or no vision in low light from an early age but retained some limited visual function in good lighting conditions. These individuals were selected because whilst they had advanced retinal degeneration they retained a limited degree of residual retinal function and might be expected to benefit from intervention. The subject characteristics at baseline are shown in Table 1A.

TABLE 1a
Subject characteristics at baseline

| | | | | | | | ERG | | |
|---|---|---|---|---|---|---|---|---|---|
| | | | | | VA | | Rods | Cones | Macula |
| | Age | | | Amino acid | (Log | Refractive | (Bright | (30 Hz | (PERG/ |
| Sub. | (Yrs) | Sex | Mutation | change | MAR) | error | flash) | flicker) | Multifocal) |
| #1 | 23 | M | homozygous | p. Tyr368His | 1.16 | −3.75/−0.50 × 170 | No definite | Residual | Undetectable |
| | | | c. | | | | response | | |
| | | | [1102T > C] | | | | | | |
| #2 | 17 | F | c. | Splice site | 1.52 | +1.50/−1.00 × 90 | Residual | Very | Untestable |
| | | | [11 + 5G > A] + | p. Gly40Ser | | | | reduced & | (nystagmus) |
| | | | [118G > A] | | | | | delayed | |
| | | | | | | | | (4.0 μV; | |
| | | | | | | | | 41 ms) | |
| #3 | 18 | M | c. [16G > T] + | p. [Glu6X] + | 0.76 | −0.25/−2.00 × 52 | No definite | Very | Undetectable |
| | | | [499G > T] | [Asp167Tyr] | | | response | reduced & | |
| | | | | | | | | delayed | |
| | | | | | | | | (10 μV; | |
| | | | | | | | | 42 ms) | |

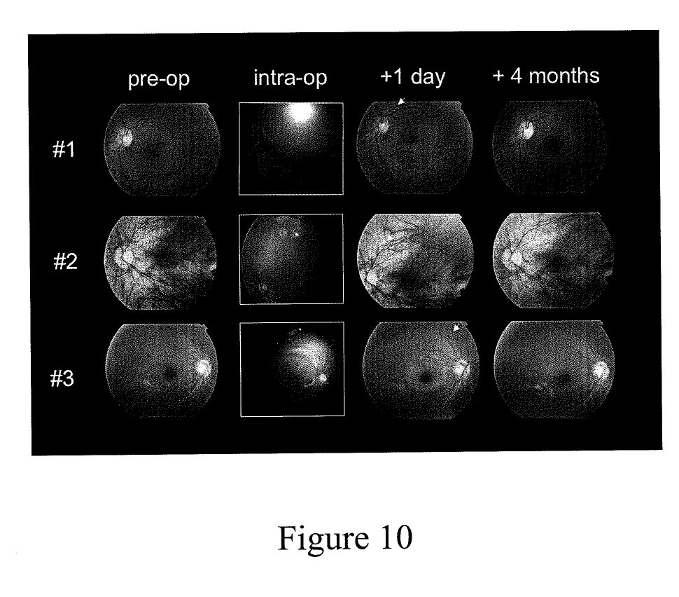

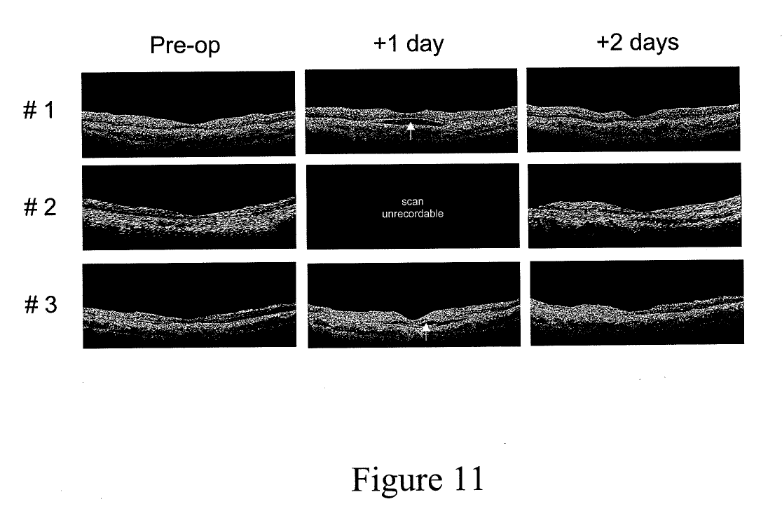

The vitrectomy and subretinal injection of vector was performed as outlined above without complication in each subject. The vitreous gel was relatively degenerate; a posterior vitreous detachment was present in subject #2 and readily induced in subjects #1 and #3 by active aspiration at the optic disc using the vitreous cutter. To deliver vector to the relatively well-preserved retina at the posterior pole, we made a retinotomy superior to the proximal part of the superotemporal vascular arcade. To minimize injection of vector into the vitreous or choroid, we first induced a small detachment of the neurosensory retain using Hartman's solution before injecting up to 1 ml rAAV vector (thus creating a “bleb”) via the same single retinotomy. In subject #2 the bleb of vector extended spontaneously across the macula. In subjects #1 and #3 we actively manipulated the bleb to involve the macula by injecting air into the vitreous cavity. We caused no iatrogenic retinal tears. We left the vector in-situ under fluid without retinopexy or intraocular tamponade. On clinical examination 24 hours postoperatively the induced retinal detachment had almost fully resolved in each case (FIG. 10). OCT demonstrated minimal persistent sub retinal fluid at the macula that resolved 2-3 days postoperatively (FIG. 11). Visual acuity loss associated with the temporary retinal detachment induced by vector administration had improved to pre-operative levels by 6 months.

We detected no dissemination of vector by PCR amplification of vector genomes isolated from samples of tears, serum and saliva collected 1 day and 30 days following vector administration, or from semen collected at 30 days. We observed mild, self-limiting post-operative intraocular inflammation that typically follows vitrectomy surgery. We found no evidence of cystoid macular edema clinically or by ocular coherence tomography. We detected no specific cellular or humoral immune responses specific to AAV capsid or specific humoral reponses to transgene product. We detected a small increase in non-specific activation of T-cells in 2 subjects, consistent with a rebound in the numbers of some lymphocyte subsets after withdrawal of corticosteroids. We observed no adverse effects on visual function in any subject. As shown in Table 1B, in each case, visual acuity and contrast sensitivity was maintained (LogMAR is the logarithm of the minimum angle of resolution and was used here because it allows comparisons of visual acuity scores more precisely than Snellen acuity).

TABLE 1b
Visual Acuities (LogMAR) and Contrast Sensitivities (LogCS).

| | | | 2 months | 4 months | 6 months | 12 months |
|---|---|---|---|---|---|---|
| Subject | | Baseline | post-op | post-op | Post-op | post-op |
| #1 | Study eye LogMAR | 1.16 | 1.06 | 0.98 | | 0.86 |
| | Control eye LogMAR | 0.88 | 0.90 | 0.68 | | 0.78 |
| | Study eye LogCS | 0.05 | 0.30 | 0.60 | | 0.50 |
| | Control eye Log CS | 0.55 | 0.60 | 0.55 | | 0.55 |
| #2 | Study eye LogMAR | 1.52 | 1.50 | 1.58 | | 1.52 |
| | Control eye LogMAR | 1.62 | 1.56 | 1.52 | | 1.58 |
| | Study eye LogCS | 0.00 | 0.00 | 0.00 | | 0.00 |
| | Control eye Log CS | 0.00 | 0.00 | 0.00 | | 0.00 |
| #3 | Study eye LogMAR | 0.76 | 0.90 | 0.80 | 0.76 | |
| | Control eye LogMAR | 0.54 | 0.46 | 0.40 | 0.44 | |
| | Study eye LogCS | 0.85 | 0.35 | 0.45 | 0.60 | |
| | Control eye Log CS | 1.10 | 1.10 | 1.20 | 1.10 | |

We detected no significant loss of retinal sensitivity on microperimetry, manual dynamic perimetry or automated static perimetry. We detected no significant change in retinal responses to flash or pattern electroretinography.

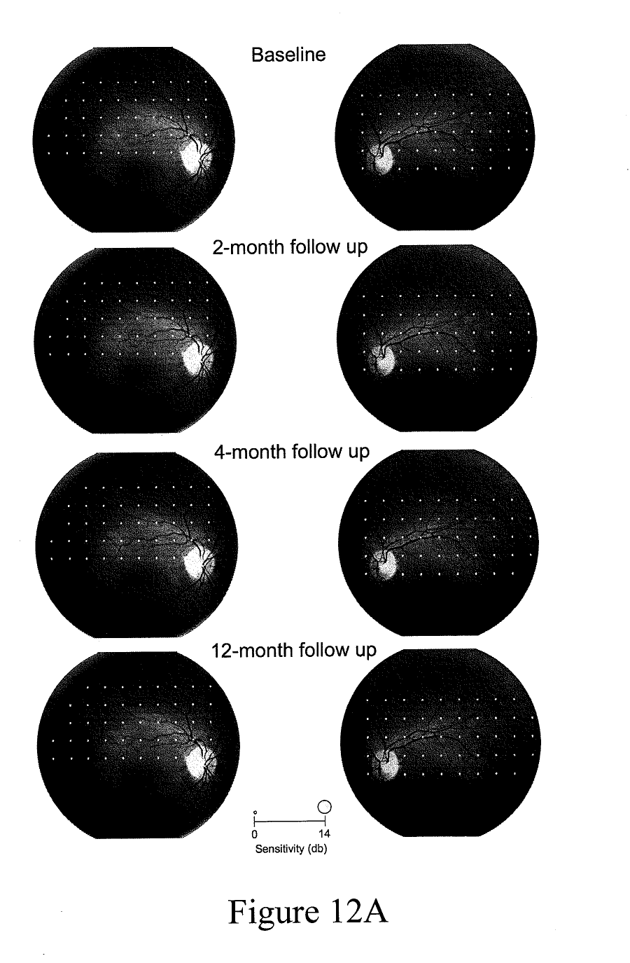

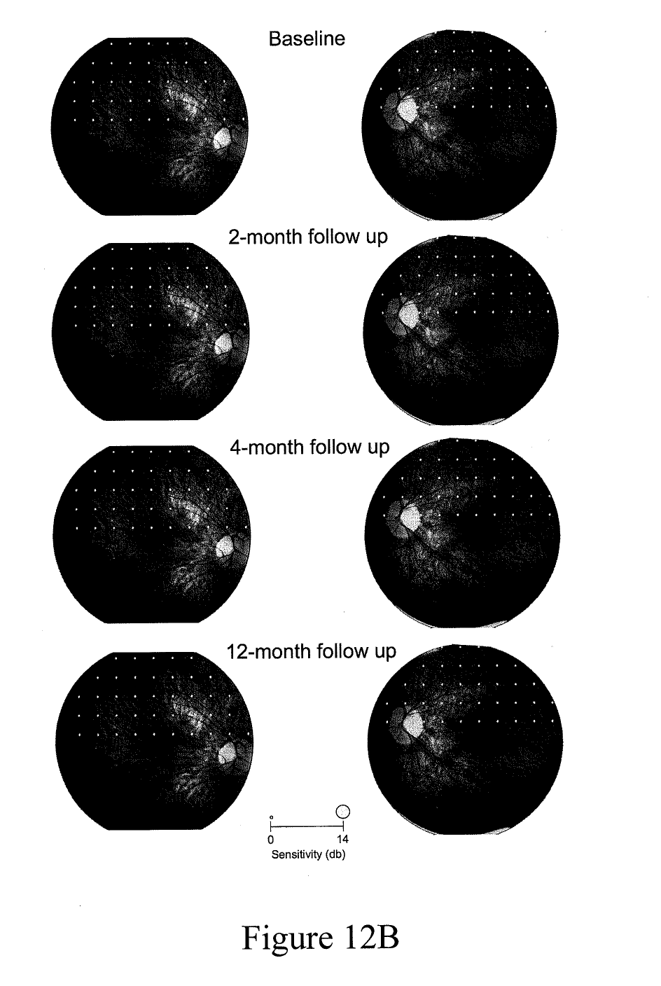

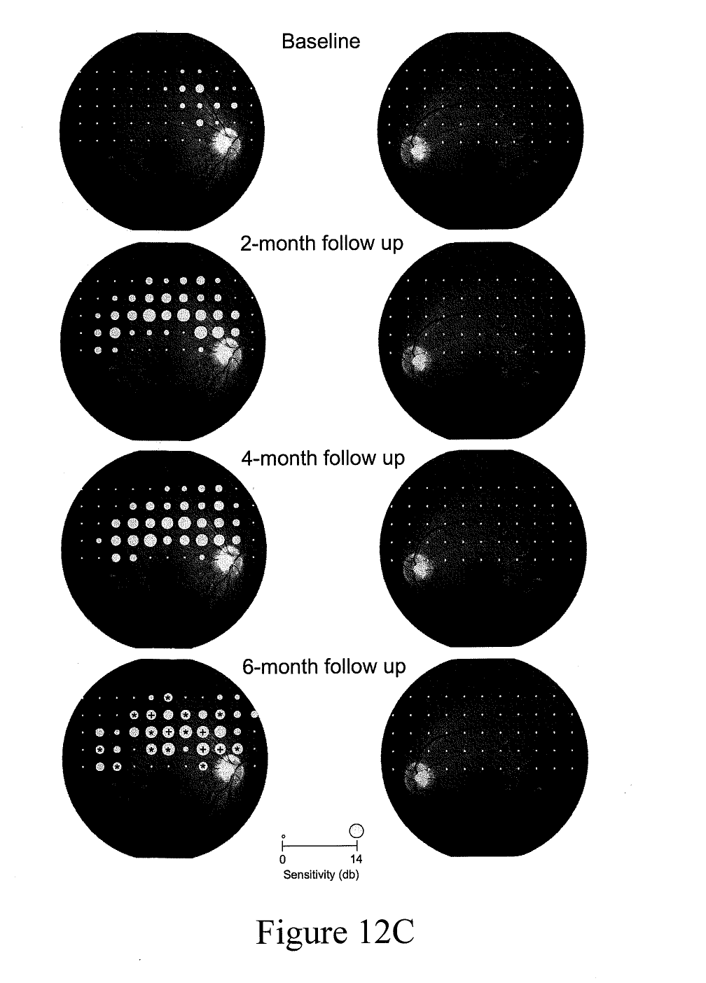

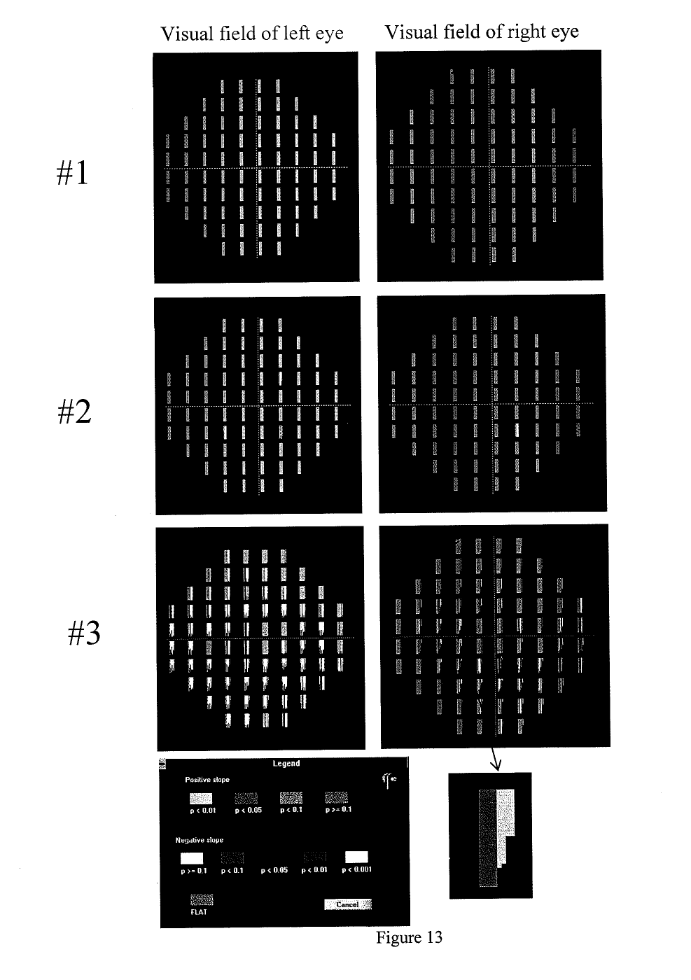

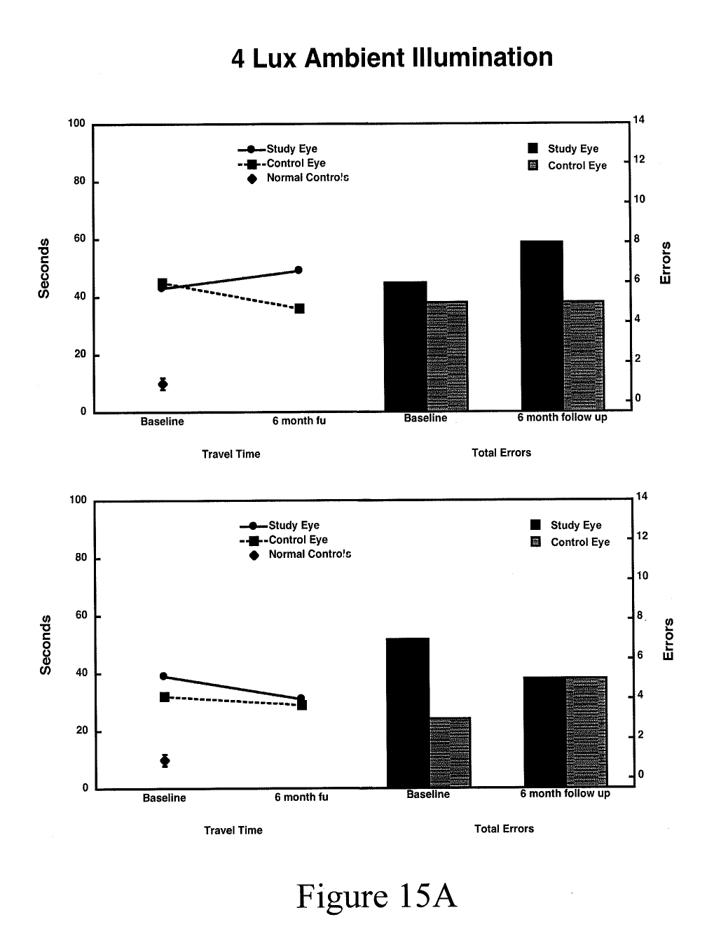

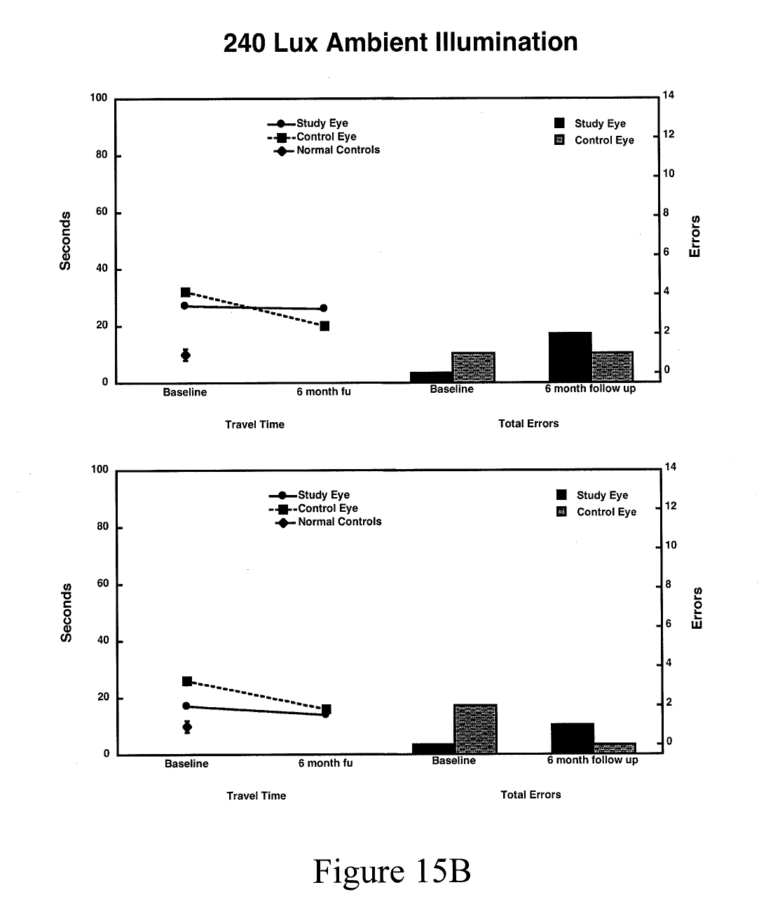

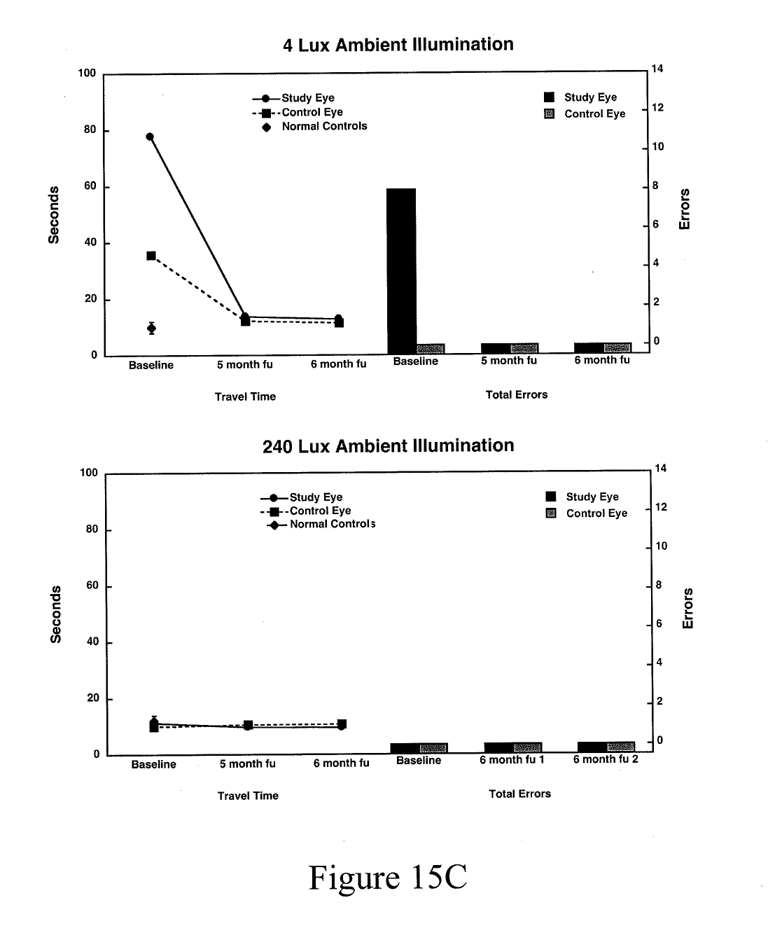

In subject #3 we found significant improvements in retinal sensitivity following vector administration. By microperimetry we measured a consistent increase of up to 14 db retinal sensitivity within an area at the posterior pole extending from the outer macula to beyond the major vascular arcade (FIG. 12C). By dark-adapted perimetry we measured a progressive improvement in retinal sensitivity across the same area of retina (FIG. 13). Furthermore, in the same subject we observed a significant improvement in visual mobility in low light conditions (FIG. 15C). The time taken to navigate a course at 4 lux improved from 77 seconds to 14 seconds and the number of navigational errors was reduced from 8 to 0 following administration of vector.

### Results

Fundus Appearance of Study Eyes Before and after Vector Administration.

FIG. 10 shows colour fundus photographs showing appearance of the retina in each subject prior to vector administration (pre-op), immediately following subretinal vector injection (intra-op) and at 1 day and 4 months postoperatively. Subretinal injection of vector resulted in bullous detachment of the neurosensory retina from the retinal pigment epithelium. One day postoperatively the induced retinal detachment had almost completely resolved; the site of vector injection is indicated in each case (white arrows). We observed no change in fundus appearance subsequently.

Optical Coherence Tomography (OCT) Images of the Maculae in Study Eyes Before and after Vector Administration.

Cross-sectional images of the retina were acquired using the Stratus OCT3 scanner (STRATUS OCT™ Optical Coherence Tomography, Carl Zeiss Meditec Inc, Dublin, Calif., USA). Six 6 mm radial line scans were obtained using a standardised mapping protocol to demonstrate the structure of the retinal layers in each subject. The appearance of the retina at the macula is demonstrated pre-operatively and at 1 day and 2 days post-operatively (FIG. 11). The presence of residual subretinal vector 1 day postoperatively is demonstrated (arrows) in subjects #1 and #3 by an area of low signal (black). Images in subject #2 at 1 day postoperatively were unrecordable owing to high-amplitude nystagmus. There was no evidence of residual subretinal fluid after 2 days in any of the 3 subjects. The appearances were unchanged for the duration of follow-up (up to 12 months).

### Assessment of Visual Function of Control and Study Eyes by Microperimetry.

Microperimetry was performed using a Nidek MP1 microperimeter (NAVIS software version 1.7.1, Nidek Technologies, Padova, Italy). Following 10 minutes of dark adaptation a white fixation cross (31.8 cd/m²) was displayed on a dim background (1.27 cd/m²). Goldmann V stimuli of 200 ms duration and a maximum luminance of 127 cd/m² were presented with a 4-2 adaptive staircase thresholding strategy. All testing followed a standardised, detailed protocol, with controlled room lighting, dark-adaptation period and a fixed sequence of test patterns. The test was fully automated so there was little opportunity for experimenter bias. The subject's eye position was continually monitored by an infrared camera and the microperimeter tracks the eye movements to compensate for shifts in the direction of gaze. Reliability parameters were determined for each test including fixation losses, false negative and false positive responses. Test reliability was assessed by projecting a light onto a known blind spot (the optic nerve head); positive responses to the light indicate poor reliability. FIGS. 12A and 12B show data for subject #1 and subject #2 respectively. There were no changes in retinal sensitivity in these subjects. The top row of FIG. 12C shows data for the right and left eyes of subject #3 at baseline (the average of two measurements taken 1 week apart). The second through fourth rows show results for the 2-month, 4-month, and 6-month follow-up examination. Measurements were performed on the same retinal loci by registering the fundus image with the baseline image. The size of the circular symbols indicates retinal sensitivity on a 0±14 dB scale. In an area extending from the outer macula to beyond the major vascular arcade (including the inner macula) the retinal sensitivity improved in the right (study) eye by as much as 14 dB (a factor of 25). This means the subject could see small spots of light that were 1/25 as bright compared with before treatment. There was no improvement in the left (control) eye. Change in sensitivity at each tested location from baseline to 6-month follow up was evaluated with pointwise linear regression. Of the 55 locations tested, 12 had significant positive slopes (p<0.05) ranging from 12±28 dB/year and are marked with an asterisk (*) in the lower left panel. We would expect no more than 3 points to pass this test by chance alone. In addition, there were 5 locations which showed a step change in sensitivity of 9 dB or greater (an 8-fold increase in sensitivity) that was sustained at 4-month and 6-month follow up. These locations are marked with a plus (+).

### Assessment of Visual Function by Dark-Adapted Perimetry

In dark-adapted conditions using a short wave length stimulus, sensitivities were measured at 76 locations in the central visual field using a modified Humphrey perimeter. Measurements were made between 60 and 240 minutes dark adaptation using Goldmann V stimuli of 200 ms duration. All testing followed a standardised, detailed protocol, with controlled room lighting, dark-adaptation period and a fixed sequence of test patterns. The test was fully automated so there was little opportunity for experimenter bias. The subject's eye position was continually monitored by an infrared camera. Reliability parameters were determined for each test including fixation losses, false negative and false positive responses. Test reliability was assessed by projecting a light onto a known blind spot (the optic nerve head); positive responses to the light indicate poor reliability. Analysis of the results was made using Progressor' software that provides significance levels for change over time at each individual test location which represents a series of 8 measurements over the 6-month follow up. The lengths of the bars represent sensitivity, with long bars showing sensitivity loss and short bars normal sensitivity. Yellow indicates a decline in sensitivity which is not statistically significant, red indicates a decline in sensitivity that is significant at less than 0.05, whilst green indicates an improvement in sensitivity that is statistically significant at p<0.01 (color not shown). One example, at the X/Y co-ordinate (−9,+3) of the right eye of subject #3, is magnified: this showed the sensitivity measurements going from baseline on the left sequentially through the follow-up assessments on the right. In this example, the long grey bars on the left indicated that the subject was unable to see the light stimulus at maximum intensity. The shorter bars on the right indicated progressive improvement in sensitivity that was statistically significant at p<0.01. In subjects #1 and subject #2 there was not a single location which showed a statistically significant change at the level of p<0.05 for either improvement or deterioration. In subject #3, the left (control) eye showed some locations with yellow and red bars showing decline in sensitivity that did not reach significance better than 0.05. By contrast, in the right (study) eye 37 locations showed significant improvements in sensitivity at p<0.01. The mean sensitivity at 9 locations in the inferonasal region improved from 4 dB at baseline to 26 dB after treatment while 9 locations in the inferotemporal quadrant improved from 7 dB to 28 dB. This was equivalent to an improvement in sensitivity of greater than 20 dB or a factor of over 100 times improved sensitivity.

### Assessment of Visual Mobility

Visually guided mobility was assessed at the UCL Pedestrian Accessibility and Movement Environment Laboratory (PAMELA). PAMELA is a unique mobility research facility (in a specially designed, converted warehouse building) that incorporates a sophisticated set of monitoring and data collection systems including starlight video cameras, laser scanners which can locate objects in the laboratory within 1-2 cm, eye tracking systems and heart rate monitors. To ensure consistent light levels the illumination of the platform was measured before and after testing, and found to vary by less than 5% overall and less than 3% in the critical area of the mobility maze. Dark adaptation time was held constant across sessions and the maze was randomly configured for each test. The experimenter, though not masked to the treatment eye, stood behind the subject and did not speak to him except to read instruction from a printed script. Visual mobility was tested with a 10.8 m×7.2 m raised platform with concrete paving assessed stones that were configured to simulate an outdoor pavement. Subjects negotiated a 13 m long maze with 8 moveable barriers (1.8 m×1.2 m) painted in colours matching light or dark blue denim, and the entire platform area was illuminated from overhead to calibrated light levels ranging from 240 lux (moderate office lighting) to 4 lux (UK night time pedestrian lighting standard).

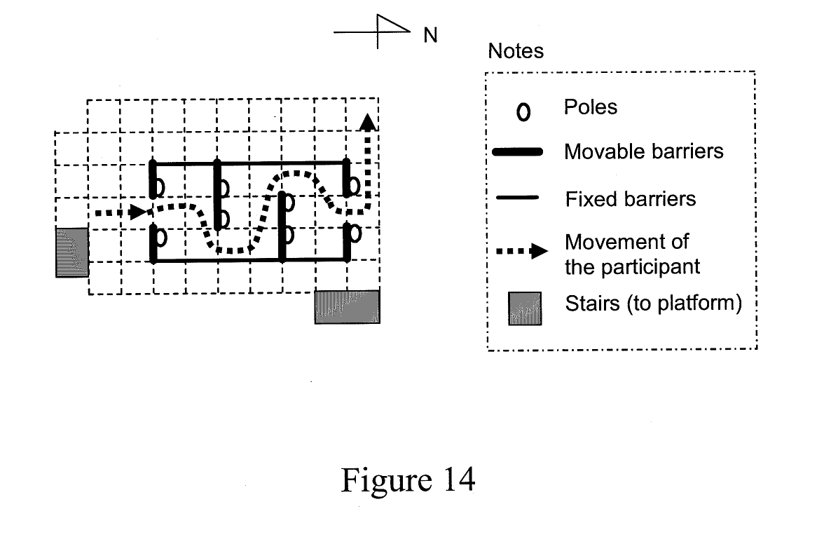

Testing was monocular with the fellow eye occluded by an opaque eye patch. The subject wore a helmet-mounted eye tracker that followed the direction of gaze. The subject was positioned at one end of the maze and instructed to walk through at a normal comfortable pace without touching the barriers. The experimenter followed along just behind to ensure the subject's safety. Total travel was recorded with a stopwatch along with mobility errors (touching a barrier, loss of orientation). The barriers were randomly re-positioned before each run and the subject was given 15 minutes to adapt to changes in illumination levels. FIG. 14 shows a schematic of the layout of the platform and one configuration of the maze. FIG. 15A-15B show data at 4 lux and 240 lux for subjects #1 and #2. Neither subject showed an improvement in mobility performance with the study eye following treatment. FIG. 15C showed data for subject #3 at 4 lux and 240 lux illumination levels. Average travel times (±1 S.D.) for 5 control subjects are indicated (10 (±2) seconds). Control subjects made no errors. In bright conditions, subject #3's performance was within normal limits at baseline and follow up. Under low illumination at baseline subject #3 performed very poorly with the study eye compared to the control eye with which he made no errors. At follow up we observed a small change for the control eye. We attributed this to a general learning effect; a similar improvement in travel time under dim illumination was also observed in another subject. However, subject #3's travel time improved following vector administration from 77 seconds to 14 seconds for the study eye and mobility errors (bumps or losses of orientation) declined from 8 to 0, Similar results were obtained in a second follow up visit four weeks later.

### Summary of Clinical Effects

Table 12 shows a summary of the aggregate clinical effects of the procedure on subjects #1-3.

TABLE 12
Aggregate summary of clinical effects on subjects #1-3.

| | Adverse | No difference | Improvement |
|---|---|---|---|
| | The following are measures of | | |
| | adverse outcome | | |
| Fundoscopy: | | | |
| AC cells | | X | |
| Vitreal cells | | X | |
| Media | | X | |
| Retinal morphology | | X | |
| | The following are measures of both | | |
| | adverse outcome (worsening) and | | |
| | of functional improvements | | |
| Visual acuity: | | | |
| Subjective vision | | X | |
| Visual acuity | | X | |
| Low light VA | | X | |
| MN read acuity | | X | |
| MN read CPS | | X | |
| Max read rate | | X | |
| Pelli Robson | | X | X |
| Visual mobility | | X | X |
| OCT | | | |
| Central retinal thickness | | X | |
| Microperimetry | | X | X |
| Automated perimetry/ | | X | X |
| Fine matrix mapping | | | |
| Goldmann perimetry | | X | |
| Flicker sensitivities | | X | |
| Autofluorescence | | X | |
| Electrophyiology | | | |
| VEP (pattern reversal) | | X | |
| VEP (flash) | | X | |
| Pattern ERG | | | |
| ERG (rod specific) | | | |
| Bright flash ERG | | X | |
| 30 Hz flicker | | X | |
| Photopic single flash ERG | | X | |
| Photopic ON and OFF | | X | |
| responses | | | |
| S-cone ERG | | X | |
| Multifocal ERG | | X | |

Elispot Assay to Detect Cytokine Expression in PBMCs in Response to Co-Culture with AAV2.

None of the subjects displayed a specific increase in the number of IFNγ secreting cells following co-culture of PBMCs with intact AAV2 particles. PVDF plates were coated with anti-IFNγ and blocked with FCS. PBMC were resuspended in serum-free media and 2.5×10e5 cells added per well. Positive controls contained anti-CD3 and negative controls were incubated in media only. Triplicate test wells contained 10e7 intact vector particles. Plates were incubated at 37° C. for 48 h. Plates were developed using an avidin-alkaline phosphatase detection system following a biotinylated detection antibody. The data shows the average spots per well of triplicate test wells ±SD (data not shown). Test wells with more than double the number of spots in the negative controls were considered to be a positive response against antigen. This assay shows that AAV2-specific T-cell responses were not generated by the subjects' PBMCs following co-culture with the AAV2 vector. An increase in non-specific activation was observed in subject #1 and subject #3 at week 8 and 16. This is consistent with a rebound in the numbers of some lymphocyte subsets after removal of steroids. The effects observed at week 52 in subject #1 may be due to a respiratory infection the patient had suffered 2-3 weeks before the blood sample was taken. The increased activation observed in the negative control well indicates the increase in IFNγ expression was not in response to AAV2.

Elisa to Detect Circulating Levels of IgA, IgG and IgM Against AAV2 or rRPE65.

None of the subjects showed any increase in circulating IgA, IgG or IgM against either vector or transgene. Serum was isolated from the same sample used for PBMC isolation and tested at the same time points for antibodies against AAV2 and recombinant human RPE65. Microtitre plates were coated with 50,000 vp/well AAV2 or 0.5 μm/ml rRPE65 then blocked with 50% goat serum in PBS. Serum dilutions were added to wells (replicates of 6) and incubated for 2 h. Plates were incubated with anti-human IgA, IgG, IgM, then developed with TMB substrate. Data is expressed as the relative antibody titre compared with baseline samples obtained before vector administration and run alongside each assay (data not shown).

### Neutralising AAV-2 Antibody Titre.

None of the subjects show an increase in humoral responses to AAV2. Serum was assayed for the ability to block the transduction of 293T cells with AAV2-GFP. Serum was serially diluted in multi-well plates using DMEM. AAV2-GFP was added to each well and plates were incubated at 37° C. for 1 h before addition to 293T cells in triplicate. The NAb titre was expressed as the serum dilution that resulted in 50% inhibition of transduction by AAV2-GFP. Baseline serum from each subject was assayed alongside each post-op sample. Green cells were counted in the test wells after 48 h and compared with the number of green cells in the baseline serum sample. Subject #1 showed a baseline titre of 1:4, and this remained at around the same level 1 year after vector administration. Subjects #2 and #3 had undetectable levels of NAb before vector delivery, and the titre remained undetectable for the duration of follow-up.

### Analysis of the Subjects' Safety

The details for each subject from the annual safety report from 1 Jan. 2007 to 21 Dec. 2007 are reported below.

### Subject 1.

The first trial subject (RJ) had Autosomal Recessive Retinitis Pigmentosa, with a mutation homozygous for c.1102T>C p.Tyr368H is mutation in exon 10 of the RPE65 gene.

The subject was enrolled 29 Jan. 2007 and underwent uncomplicated surgery on the left eye, with delivery of 0.9 ml of viral suspension (1×10¹¹ vg/ml) to 30% of retinal area (superior retina and posterior pole) on 7 Feb. 2007. Vitreous Fluid/Air exchange was performed.

AEs were as follows: (1) Transiently raised IOP, controlled by topical ocular hypotensive. (2) Mild mood disturbance (lethargy) for 2 weeks following procedure. Believed to be related, in part, to oral prednisolone. (3) Transient glycosuria, during prednisolone administration, not associated with hyperglycaemia.

No AR, SAE, SAR, USAR, SSAR, or SUSAR occurred.

Clinical findings are shown in Table 1.

Test results (acuity, reading speed, contrast sensitivity, OCT, serology, and biodistribution) for the operated eye are shown in Tables 2-4. Microperimetry: No decline in performance at 8 weeks, 4 months, or 12 months post-surgery. Automated Perimetry/Fine Matrix Mapping: Poor peripheral vision at baseline (photopic and scotopic). No decline in performance and no improvement at 8 weeks, 4 months and 12 months after surgery. Goldmann Perimetry: No decline in performance and no improvement at 8 weeks or 4 months after surgery, compared to baseline. Flicker sensitivities: No decline in performance and no improvement at 8 weeks, 4 months, or 12 months after surgery, compared to baseline. Autofluorescence: No evidence of autofluorescence at baseline or at 8 weeks, 4 months or 12 months post-surgery. Electrophysiology: No decline in performance and no improvement at 8 weeks, 4 months or 12 months after surgery, compared to baseline: VEP (pattern reversal) no definite response seen; VEP (flash)—4.0 uV (no latency); Pattern ERG—undetectable; ERG (rod specific)—undetectable; Bright flash ERG—no definite response; 30 Hz flicker—v. low amplitude (5 uV) response both eyes; Photopic single flash ERG—barely detectable; Photopic ON and OFF responses—not definitely detected; S-cone ERG—not definitely detected; Multifocal ERG—no definite response. Visual Mobility: No change in performance seen compared to baseline.

TABLE 1
Clinical Findings (operated eye; subject 1)

| | | Day 1 | | | | |
|---|---|---|---|---|---|---|
| Visit: | baseline | post-op | Day 2 | Day 5 | Day 7 | Wk 2 |
| Symptoms: | — | Sl. | No dysuria/ | Tiredness, | Intermittent | Headache/ |
| | | discomfort | frequency | no urinary | headache/ | tiredness/ |
| | | LE, dysuria/ | | symptoms | tiredness | back pain |
| | | frequency | | | | |
| Δ vision | — | ‘Darker’, sl. | Central | Near pre-op | LE≈RE | At pre-op |
| (subj.): | | distortion | vision | level, | (occ. | level, eye |
| | | | brighter | ↓ distortion | distortion) | comfortable |
| VA* | 1.16 | CF* | CF* | CF* | CF* | CF* |
| IOP | 13 | 28 | 25 | 24 | 28 | 34 |
| (mm/Hg) | | | | | | (g.timolol |
| | | | | | | 0.25% bd |
| | | | | | | started) |
| AC cells: | Nil | + | + | + | + | + |
| Vit cells: | Nil | + | + | +/− | − | +/− |
| Media | Clear | Clear | Clear | Clear | Clear | Clear |
| Retina: | Flat, sl. | Shallow SRF | Attached, no | Attached, | Attached, | Attached, |
| | rotated | at macula | inflam | no inflam | no inflam | no inflam |
| | macula, | | | | | |
| | no ↑ | | | | | |
| | pigment | | | | | |
| Urine dip | −ve | Glucose | — | — | Glucose ++ | −ve |
| | | ++++ | | | | |
| BP | 118/80 | 110/80 | — | — | 110/70 | 120/75 |
| BM | 4.2 | 5.3 | — | 4.8 | 5.6 | 6.6 |

| Visit: | Wk 3 | Wk 4 | Wk 8 | Month 4 | Month 8 | Month 12 |
|---|---|---|---|---|---|---|
| Symptoms: | Feeling | Feeling well | None | None | None | None |
| | better | | | | | |
| Δ vision | At pre-op | Fully at | At pre-op | Slight | At pre-op | At pre-op |
| (subj.): | level, | pre-op level | level | yellow tint | level | level |
| | sl. ↓ | | | to vision in | | |
| | contrast | | | last 2 wks | | |
| VA* | CF* | CF* | 1.06 | 0.98 | 1/60* | 0.88 |
| IOP | 14 | 18 | 11 | 15 | 14 | 12 |
| (mm/Hg) | (still | | | | | |
| | g.timolol | | | | | |
| | 0.25% bd) | | | | | |
| AC cells: | +/− | − | − | − | − | − |
| Vit cells: | − | − | − | − | − | − |
| Media | Clear | Clear | Clear | Clear | Clear | Clear |
| Retina: | Attached, | Attached, | Attached, | Attached, | Attached, | Attached, |
| | no inflam | no inflam | no inflam | no inflam | no inflam | no inflam |
| Urine dip | −ve | −ve | — | — | — | — |
| BP | 100/70 | 120/80 | — | — | — | — |
| BM | 6.1 | 5.0 | — | — | — | — |

*LogMAR assessed at baseline, 8 wks, 4 and 12 month

TABLE 2
Test results (operated eye; subject 1): Acuity, reading speed, contrast sensitivity

| | | Day 1 | | | | | | | | | |
|---|---|---|---|---|---|---|---|---|---|---|---|
| Visit: | Baseline | post-op | Day 2 | Day 5 | Day 7 | Wk 2 | Wk 3 | Wk 4 | Wk 8 | Month 4 | Month 12 |
| VA (LogMAR@2 m) | 1.16 | — | — | — | — | — | — | — | 1.06 | 0.98 | 0.88 |
| LLVA | 1.82 | — | — | — | — | — | — | — | 1.48 | 1.40 | 1.84 |
| (LogMAR@½ m) | | | | | | | | | | | |
| MN read acuity | 1.31 | — | — | — | — | — | — | — | 1.06 | 1.06 | 0.9 |
| (LogMAR) | | | | | | | | | | | |
| MN read CPS | 1.4 | — | — | — | — | — | — | — | 1.1 | 1.4 | 1.1 |
| (LogMAR) | | | | | | | | | | | |
| Max. read rate | 1.4 | — | — | — | — | — | — | — | 1.1 | 1.4 | 1.2 |
| (LogMAR) | | | | | | | | | | | |
| Pelli-Robson (LogCS) | 0.05 | — | — | — | — | — | — | — | 0.3 | 0.6 | 0.5 |

TABLE 3
Test results (operated eye; subject 1): OCT

| | | Day 1 | | | | |
|---|---|---|---|---|---|---|
| Visit: | Baseline | post-op | Day 2 | Day 5 | Day 7 | Wk 2 |
| Central | 200 +/− 30 | 307 +/− 17 | 128 +/− 20 | 1 32 +/− 17 | 154 +/− 45 | 191 +/− 47 |
| retinal | (188 +/− 15) | (142 +/− 10) | (197 +/− 55) | (181 +/− 49) | (180 +/− 25) | (183 +/− 37) |
| thickness | | | | | | |
| (um) | | | | | | |
| Visit: | Wk 3 | Wk 4 | Wk 7/8 | Month 4 | Month 8 | Month 12 |
| Central | 209 +/− 37 | 143 +/− 15 | 150 +/− 8 | 156 +/− 9 | 141 +/− 16 | 180 +/− 28 |
| retinal | (180 +/− 46) | (189 +/− 45) | (190 +/− 41) | (177 +/− 32) | (148 +/− 11) | (160 +/− 3) |
| thickness | | | | | | |
| (um) | | | | | | |

TABLE 4
Test results (operated eye; subject 1): serology, biodistribution

| | | Day 1 | Day | Day | | | | | | | |
|---|---|---|---|---|---|---|---|---|---|---|---|
| Visit: | Baseline | post-op | 2 | 5 | Day 7 | Wk 2 | Wk 3 | Wk 4 | Wk 7/8 | Month 4 | Month 12 |
| FBC | NAD | ↑WBC | — | — | ↑WBC | — | ↑WBC | NAP | — | — | — |
| (1 × | | 11.1 (4.0-11.0) | | | 11.2 (4.0-11.0) | | 11.6 | | | | |
| 10e9/l) | | ↑Neutrophils | | | ↑Neutrophils | | (4.0-11.0) | | | | |
| | | 10.0 (1.5-7.0) | | | 10.0 (1.5-7.0) | | ↑Neutrophils | | | | |
| | | ↓Lymphocytes | | | ↓Lymphocytes | | 9.2 (1.5-7.0) | | | | |
| | | 0.8 (1.2-3.5) | | | 0.7 (1.2-3.5) | | | | | | |
| U + E | NAD | NAD | — | — | NAD | — | NAD | NAD | — | — | — |
| LFT | Total | Total bilirubin | — | — | NAD | — | NAD | NAD | — | — | — |
| (umol/l) | bilirubin | 19 (0-16) | | | | | | | | | |
| (g/l) | 17 (0-16) | Albumin | | | | | | | | | |
| | | 53 (40-52) | | | | | | | | | |
| Il-4,5 | No significant | No | — | — | No | No | No | No | No | No | No |
| induction | difference | significant | | | significant | significant | significant | significant | significant | significant | significant |
| | cf. −ve control | increase | | | increase | increase | increase | increase | increase | increase | increase |
| IFN-γ | No significant | No | — | — | No | No | No | No | No | No | No |
| induction | difference | significant | | | significant | significant | significant | significant | significant | significant | significant |
| | cf. −ve control | increase | | | increase | increase | increase | increase | increase | increase | increase |
| ↑Anti- | Low levels | No | — | — | No | No | No | No | No | No | No |
| AAV | (within control | significant | | | significant | significant | significant | significant | significant | significant | significant |
| Abs | range) | increase | | | increase | increase | increase | increase | increase | increase | increase |
| ↑Anti- | None | None | — | — | None | None | None | None | None | None | None |
| RPE | detected | detected | | | detected | detected | detected | detected | detected | detected | detected |
| Abs | | | | | | | | | | | |
| Vector | None | None | — | — | — | — | — | None | — | — | — |
| genome | detected | detected | | | | | | detected^{†} | | | |
| in tissues* | | | | | | | | | | | |

^{†}semen not provided
*blood plasma, saliva, tears

### Subject 2

The second trial subject (LM) had Autosomal Recessive Retinitis Pigmentosa, with a compound heterozygous mutation c. [11+5G>A]+[118G>A] p. Gly40Ser. The subject was enrolled 1 Feb. 2007 and underwent uncomplicated surgery on the left eye, with delivery of 0.9 ml of viral suspension (1×10¹¹ vg/ml) to 30% of retinal area (superior retina and posterior pole) on 25 Apr. 2007. Vitreous Fluid/Air exchange was not performed, but rather the weight of the bleb allowed repositioning without need for fluid/air exchange.

At eight weeks post-surgery no Adverse Events or Adverse Reactions have occurred. A moderately raised white cell count was noted in the first two weeks post-surgery, consistent with steroid-induced mobilisation of bone-marrow neutrophils.

No AR, SAE, SAR, USAR, SSAR, or SUSAR occurred.

Clinical findings are reported in Table 5.

Test results (acuity, reading speed, contrast sensitivity, OCT, serology, and biodistribution) for the operated eye are shown in Tables 6-8. Microperimetry: No decline or improvement in performance seen at 2 or 4 months post-surgery. Automated Perimetry/Fine Matrix Mapping: Poor peripheral vision at baseline (photopic and scotopic conditions). No decline or improvement in performance seen at 2 or 4 months post-surgery. Flicker sensitivities: No decline or improvement in performance seen at 2 or 4 months post-surgery, compared to baseline. Autofluorescence: No evidence of autofluorescence at baseline, 3 or 4 months post-surgery. Electrophysiology: No decline or improvement in function seen at 8 weeks, 2 months, or 4 months post-surgery, compared to baseline: Pattern ERG nystagmus precluded investigation; ERG (rod specific)—undetectable; Bright flash ERG—‘suspicion of residual activity with a-wave of approximately 5.0 uV’; 30 Hz flicker—some activity present with markedly delayed peak time (RE worse than LE: RE amplitude 3.5-4.0 uV, LE amplitude 4.5-5.0 uV); Photopic single flash ERG—not definitely detectable; Multifocal ERG—nystagmus precluded investigation. Visual Mobility: No change in performance seen compared to baseline.

TABLE 5
Clinical Findings (operated eye; subject 2).

| | | Day 1 | | | | |
|---|---|---|---|---|---|---|
| Visit: | Baseline | post-op | Day 2 | Day 5 | Day 7 | Wk 2 |
| Symptoms: | (nystagmus) | None | Moderate | None | Sl. eye | None |
| | | | eye | | discomfort | |
| | | | discomfort | | | |
| Δ vision | — | — | — | — | Sl. less clear | At pre-op |
| (subj.): | | | | | than pre-op | level |
| VA* | 1.52 | HM* | HM* | HM* | HM* | HM* |
| IOP | 8 | 16 | 7 | 10 | 12 | 10 |
| (mm/Hg) | | | | | | |
| AC cells: | Nil | ++ | + | − | ++ | − |
| Vit cells: | Nil | − | − | − | − | − |
| Media | Clear | Clear | Clear | Clear | Clear | Clear |
| Retina: | Flat, ‘blonde | Attached, | Attached, | Attached, | Attached, | Attached, |
| | fundus’, no | no inflam | no inflam | no inflam | no inflam | no inflam |
| | ↑pigment | | | | | |
| Urine dip | −ve | — | — | — | −ve | — |
| BP | 111/84 | — | 128/66 | — | — | 110/75 |
| BM | 4.3 | — | — | — | 6.9 | 5.7 |

| Visit: | Wk 3 | Wk 4 | Wk 6 | Week 8 | Month 4 | Month 8 |
|---|---|---|---|---|---|---|
| Symptoms: | None | None | None | None | None | None |
| Δ vision | At pre-op | At pre-op | At pre-op | Slight | At pre-op | At pre-op |
| (subj.): | level | level | level | subjective | level | level |
| | | | | improvement — | | |
| | | | | inconsistent | | |
| VA* | CF* | CF* | CF* | CF* | CF* | 1/60* |
| IOP | 7 | 11 | 9 | 11 | 12 | 12 |
| (mm/Hg) | | | | | | |
| AC cells: | − | − | +/− | − | − | − |
| Vit cells: | − | − | − | − | − | − |
| Media | Clear | Clear | Clear | Clear | Clear | Clear |
| Retina: | Attached, | Attached, | Attached, | Attached, | Attached, | Attached, |
| | no inflam | no inflam | no inflam | no inflam | no inflam | no inflam |
| Urine dip | — | — | — | — | — | — |
| BP | 131/73 | 127/79 | — | — | — | — |
| BM | 4.9 | 4.7 | — | — | — | — |

*LogMAR assessed at baseline, 8 wks, 4 and 12 months

TABLE 6
Test results (operated eye; subject 2): Acuity, reading speed, contrast sensitivity

| | | Day 1 | | | | | | | | |
|---|---|---|---|---|---|---|---|---|---|---|
| Visit: | Baseline | post-op | Day 2 | Day 5 | Day 7 | Wk 2 | Wk 3 | Wk 4 | Wk 8 | Month 4 |
| VA (LogMAR@1 m) | 1.52 | — | — | — | — | — | — | — | 1.50 | 1.58 |
| LLVA (LogMAR@1/4 m) | 2.30 | — | — | — | — | — | — | — | 2.24 | 2.26 |
| MN read acuity (LogMAR) | Nil read at | — | — | — | — | — | — | — | Nil read at | Nil read at |
| | 25 cm | | | | | | | | 25 cm | 25 cm |
| MN read CPS (LogMAR) | Nil read at | — | — | — | — | — | — | — | Nil read at | Nil read at |
| | 25 cm | | | | | | | | 25 cm | 25 cm |
| Max. read rate (LogMAR) | Nil read at | — | — | — | — | — | — | — | Nil read at | Nil read at |
| | 25 cm | | | | | | | | 25 cm | 25 cm |
| Pelli-Robson (LogCS) | 0.00 | — | — | — | — | — | — | — | 0.00 | 0.00 |

TABLE 7
Test results (operated eye; subject 2): OCT (n.b. nystagmus)

| | | Day 1 | | | | |
|---|---|---|---|---|---|---|
| Visit: | Baseline | post-op | Day 2 | Day 5 | Day 7 | Wk 2 |
| Central retinal | 165 +/− 5 | — | 134 +/− 27 | 266 +/− 59 | 167 +/− 34 | 193 +/− 31 |
| thickness (um) | (130 +/− 36) | (139 +/− 27) | (139 +/− 58) | (182 +/− 45) | (209 +/− 59) | (209 +/− 67) |

| Visit: | Wk 3 | Wk 4 | Wk 7/8 | Month 4 | Month 8 |
|---|---|---|---|---|---|
| Central retinal | 170 +/− 51 | 179 +/− 35 | 155 +/− 35 | 240 +/− 26 | 141 +/− 14 |
| thickness (um) | (185 +/− 75) | (185 +/− 62) | (190 +/− 26) | (206 +/− 36) | (150 +/− 0) |

TABLE 8
Test results (operated eye; subject 2): serology, biodistribution

| | | Day 1 | Day | Day | | | | | | |
|---|---|---|---|---|---|---|---|---|---|---|
| Visit: | Baseline | post-op | 2 | 5 | Day 7 | Wk 2 | Wk 3 | Wk 4 | Week 8 | Month 4 |
| FBC | NAD | Inadequate | — | — | ↑WBC | ↑WBC | NAD | — | — | — |
| (1 × 10e9/l) | | sample | | | 23.7 (4.0-11.0) | 23.4 (4.0-11.0) | | | | |
| | | | | | ↑Neutrophils | ↑Neutrophils | | | | |
| | | | | | 20.1 (1.5-7.0) | 18.3 (1.5-7.0) | | | | |
| | | | | | ↑Monocytes | ↑Lymphocytes | | | | |
| | | | | | 1.2 (0.4-1.0) | 4.2 (1.2-3.5) | | | | |
| U + E | NAD | Inadequate | — | — | NAD | NAD | NAD | — | — | — |
| | | sample | | | | | | | | |
| LFT | NAD | Inadequate | — | — | NAD | NAD | — | — | — | — |
| (umol/l) | | sample | | | | | | | | |
| (g/l) | | | | | | | | | | |
| Il-4,5 | No significant | No | — | — | No | No | No | No | No | No |
| induction | difference cf. | significant | | | significant | significant | significant | significant | significant | significant |
| | −ve control | increase | | | increase | increase | increase | increase | increase | increase |
| IFN-γ | No significant | No | — | — | No | No | No | No | No | No |
| induction | difference cf. | significant | | | significant | significant | significant | significant | significant | significant |
| | −ve control | increase | | | increase | increase | increase | increase | increase | increase |
| ↑Anti-AAV | None detected | No | — | — | No | No | No | No | No | No |
| Abs | | significant | | | significant | significant | significant | significant | significant | significant |
| | | increase | | | increase | increase | increase | increase | increase | increase |
| ↑Anti-RPE | None detected | None | — | — | None | None detected | None | None | None | None |
| Abs | | detected | | | detected | | detected | detected | detected | detected |
| Vector | None detected | None | — | — | — | — | — | — | — | — |
| genome | | detected | | | | | | | | |
| in tissues* | | | | | | | | | | |

*blood plasma, saliva, tears

### Subject 3

The third trial subject (SH) had Autosomal Recessive Retinitis Pigmentosa, with a Compound heterozygous mutation: c. [16G>T]+[499G>T]; p. [Glu6X]+[Asp167Tyr].

The subject was enrolled 3 May 2007 and underwent uncomplicated surgery on the right eye, with delivery of 1.0 ml of viral suspension (1×10¹¹ vg/ml) to 30% of retinal area (superior retina and posterior pole) on 11 Jul. 2007. Vitreous Fluid/Air exchange was performed.

The AE that occurred were: (1) Day 1 post-op: BM 9.0; urine protein +; urine glucose +++. Attributed to high-dose prednisolone (medical opinion sought—no treatment indicated, dietary advice given; BM peaked at 10.1 on day 2, results normalised by day 5; steroid regimen continued). (2) Day 14 post-op: Two episodes of mild-moderate self-limiting epistaxis (no previous history) following clinical review, milder episode of epistaxis on day 15. Platelet count 120×10e9/1 (normal range 150-400×10e9/1) prior to commencement of prednisolone. Platelet count 134×10e9/1 on day 14. Haemodynamically stable at day 14 review. (3) Day 15: Brief episode of nausea and dizziness 1 hr after taking oral prednisolone.

No AR, SAE, SAR, USAR, SSAR, or SUSAR occurred.

Clinical findings are reported in Table 9.

Test results (acuity, reading speed, contrast sensitivity, OCT, serology, and biodistribution) for the operated eye are shown in Tables 10-12. Orthoptics: No changes seen at 2 or 4 months compared to baseline. Microperimetry: Some moderate improvement in retinal function superior to foveal region (treated area) at month 2 compared to baseline—maintained at months 4 and 6 post-op. Automated Perimetry/Fine Matrix Mapping No decline in function seen at 2, 4, or 6 months compared to baseline. Evidence for improvement in performance on scotopic perimtery. Flicker sensitivities: No decline in function seen at 2, 4 or 6 months compared to baseline. Goldmann Perimetry: No decline in function seen at 2, 4 or 6 months compared to baseline. Autofluorescence: No evidence of autofluorescence at 2, 4 or 6 months compared to baseline. Electrophysiology: No change from baseline at 4 or 6 months (verbal report). Results from Jun. 9, 2007 (no change from baseline): Pattern ERG—undetectable; Flash VEPs—clearly detectable; Rod specific ERG—bilaterally undetectable; Bright flash ERG—residual activity; 30 Hz flicker ERG—profoundly delayed (amplitudes: 10 uV RE; 10 uV LE); Photopic single flash ERG—low amplitude and marked delay; EOG—no significant data; Multifocal ERG—no definite detectable responses. Visual mobility: Evidence of improvement in visual mobility at 6 months.

TABLE 9
Clinical Findings (operated eye; subject 3)

| | | Day 1 | | | | |
|---|---|---|---|---|---|---|
| Visit: | Baseline | post-op | Day 2 | Day 5 | Day 7 | Wk 2 |
| Symptoms: | — | — | None | None | None | None |
| | | | (no polyuria/ | | | |
| | | | polydipsia) | | | |
| Δ vision | — | Vision | — | Gradually | Almost at | At pre-op |
| (subjective): | | generally | | improving | pre-op level | level |
| | | blurred RE | | | | |
| VA* | 0.76 | CF* | CF* | CF* | CF* | CF* |
| IOP | 8 | 8 | 5 | 5 | 14 | 17 |
| (mm/Hg) | | | | | | |
| AC cells: | Nil | ++ | + | + | +/− | − |
| Vit cells: | Nil | + | + | − | − | − |
| Media | Clear | Clear | Clear | Clear | Clear | Clear |
| | Flat; | Attached; | Attached; | Attached; | Attached; | Attached; |
| | mottled | no inflam | no inflam; | no inflam; | no inflam; | no inflam; |
| | RPE; no | | few fovea | few fovea | retinal folds | no retinal |
| | ↑pigment, | | retinal folds | retinal folds | resolving | folds |
| | foveae sl. | | | | | |
| | granular | | | | | |
| Urine dip | −ve | Protein + | Protein trace | NAD | — | — |
| | | Glucose +++ | Glucose −ve | | | |
| BP | 111/84 | 131/69 | 124/69 | 126/56 | 119/64 | 127/75 |
| BM | 4.3 | 9.0 | 10.1 | 6.9 | 7.4 | 5.3 |

| Visit: | Wk 3 | Wk 4 | Wk 6 | Wk 8 | Month 4 | Month 6 |
|---|---|---|---|---|---|---|
| Symptoms: | None | None | None | None | None | None |
| Δ vision | At pre-op | At pre-op | At pre-op | At pre-op | At pre-op | At pre-op |
| (subjective): | level | level | level | level | level | level |
| VA* | CF* | CF* | CF* | 0.90 | 0.80 | 0.76 |
| IOP | 11 | 27 | 6 | 7 | 7 | 8 |
| (mm/Hg) | | | | | | |
| AC cells: | − | − | − | − | − | − |
| Vit cells: | − | − | − | − | − | − |
| Media | Clear | Clear | Clear | Clear | Clear | Clear |
| | Attached; | Attached; | Attached; | Attached; | Attached; | Attached; no |
| | no inflam | no inflam | no inflam | no inflam | no inflam | inflam |
| Urine dip | — | — | — | — | — | — |
| BP | 131/73 | 109/71 | — | — | — | — |
| BM | — | 4.9 | — | — | — | — |

*LogMAR assessed at baseline, 8 wks, 4 and 12 months

TABLE 10
Test results (operated eye; subject 3): Acuity, reading speed, contrast sensitivity

| | | Day 1 | | | | | | | | | |
|---|---|---|---|---|---|---|---|---|---|---|---|
| Visit: | Baseline | post-op | Day 2 | Day 5 | Day 7 | Wk 2 | Wk 3 | Wk 4 | Wk 8 | Month 4 | Month 6 |
| VA (LogMAR) | 0.76 (0.76) | — | — | — | — | — | — | — | 0.90 | 0.80 | 0.76 |
| LLVA (LogMAR) | 1.08 (0.90) | — | — | — | — | — | — | — | 1.20 | 1.02 | 0.94 |
| MN read acuity | 1.06 (0.71) | — | — | — | — | — | — | — | 1.38 | 1.30 | 1.2 |
| (LogMAR) | | | | | | | | | | | |
| MN read CPS | 1.1 (1.0) | — | — | — | — | — | — | — | 1.4 | 1.3 | 1.3 |
| (LogMAR) | | | | | | | | | | | |
| Max. read rate | 1.2 (1.0) | — | — | — | — | — | — | — | 1.5 | 1.4 | 1.5 |
| (LogMAR) | | | | | | | | | | | |
| Pelli-Robson | 0.90 (0.85) | — | — | — | — | — | — | — | 0.35 | 0.45 | 0.60 |
| (LogCS) | | | | | | | | | | | |

(Repeated baseline results in brackets)

TABLE 11
Test results (operated eye; subject 3): OCT

| | | Day 1 | | | | |
|---|---|---|---|---|---|---|
| Visit: | Baseline | post-op | Day 2 | Day 5 | Day 7 | Wk 2 |
| Central | 130 +/− 8 | 195 +/− 17 | 122 +/− 18 | 147 +/− 22 | 115 +/− 11 | 117 +/− 26 |
| retinal | (137 +/− 9) | (138 +/− 13) | (136 +/− 12) | (136 +/− 12) | (141 +/− 10) | (144 +/− 16) |
| thickness | | | | | | |
| (um) | | | | | | |

| Visit: | Wk 3 | Wk 4 | Wk 7/8 | Month 4 | Month 6 |
|---|---|---|---|---|---|
| Central | 113 +/− 5 | 115 +/− 6 | 112 +/− 2 | 113 +/− 2 | 113 +/− 5 |
| retinal | (148 +/− 5) | (130 +/− 6) | (131 +/− 10) | (125 +/− 8) | (125 +/− 8) |
| thickness | | | | | |
| (um) | | | | | |

TABLE 12
Test results (operated eye; subject 3): serology, biodistribution

| | | | Day | Day | | | | | | |
|---|---|---|---|---|---|---|---|---|---|---|
| Visit: | Baseline | Day 1 post-op | 2 | 5 | Day 7 | Wk 2 | Wk 3 | Wk 4 | Wk 8 | Month 4 |
| FBC | ↓Platelets | ↓Platelets | — | — | ↓Platelets | ↓Platelets | — | ↓Platelets | — | — |
| (1 × 10e9/l) | 120 (150-400) | 139 (150-400) | | | 129 (150-400) | 134 (150-400) | | 125 (150-400) | | |
| | ↑Eosinophils | ↑WBC | | | ↑WBC | ↑WBC | | | | |
| | 0.8 (0.0-0.4) | 16.8 (4.0-11.0) | | | 14.2 (4.0-11.0) | 12.5 (4.0-11.0) | | | | |
| | | ↑Neutrophils | | | ↑Neutrophils | ↑Neutrophils | | | | |
| | | 13.8 (1.5-7.0) | | | 8.7 (1.5-7.0) | 10.5 (1.5-7.0) | | | | |
| | | | | | ↑Lymphocytes | | | | | |
| | | | | | 4.4 (0.4-1.0) | | | | | |
| U + E | NAD | NAD | — | — | ↑Na | NAD | — | NAD | — | — |
| (mmol/l) | | | | | 146 (135-145) | | | | | |
| LFT (umol/l) | — | NAD | — | — | NAD | NAD | — | NAD | — | — |
| (g/l) | | | | | | | | | | |
| Il-4,5 | No significant | No | — | — | No | No | No | No | No | No |
| induction | difference cf. | significant | | | significant | significant | significant | significant | significant | significant |
| | −ve control | increase | | | increase | increase | increase | increase | increase | increase |
| INF-γ | No significant | No | — | — | No | No | No | No | No | No |
| induction | difference cf. | significant | | | significant | significant | significant | significant | significant | significant |
| | −ve control | increase | | | increase | increase | increase | increase | increase | increase |
| ↑Anti-AAV | None | No | — | — | No | No | No | No | No | No |
| Abs | detected | significant | | | significant | significant | significant | significant | significant | significant |
| | | increase | | | increase | increase | increase | increase | increase | increase |
| ↑Anti-RPE | None | None | — | — | None | None | None | None | None | None |
| Abs | Detected | detected | | | detected | detected | detected | detected | detected | detected |
| Vector genome | None | None | — | — | — | — | — | None | — | — |
| in tissues* | Detected | detected | | | | | | detected | | |

*blood plasma, saliva, tears + seminal fluid (wk 4 only)

### Appraisal of Ongoing Risk: Benefit

No serious adverse effects were identified during the trial to date in any of the 3 subjects. Each subject has regained visual function to pre-operative levels following subretinal vector delivery. There has been no indication of an immune response to rAAV vector or to expressed hRPE65 protein, either clinically or by laboratory assays of serological or T-cell responses. Vector genome was undetectable in tears, serum and saliva following vector delivery.

No adverse events or adverse reactions due to the IMP were identified. Surgical delivery of vector was associated with predictable minor adverse effects. A mild transient rise in intraocular pressure (common after intraocular surgery) in subject #1 was effectively managed by appropriate additional topical medication. Minor adverse effects consistent with the effects of oral prednisolone were noted, prescribed as part of the protocol to minimise any risk of immune responses to vector administration. These included transient changes in mood (subject #1), mild hyperglycaemia (subject #3), and glycosuria (subject #1). Brief self-limiting epistaxis in subject #3 was not associated with hypertension or changes in haematological indices.

There was no evidence of deterioration in visual function or electrophysiololgy in any of the 3 subjects enrolled to date. In subject #3 there was some evidence of improved retinal function in the area of central retina exposed to the vector as assessed by both scotopic perimetry and microperimetry. This was consistent with an improvement resulting from the intervention. In subjects #1 and #3 there was a suggestion of reduced central retinal thickness following surgery as measured by ocular coherence tomography. This was associated with maintenance or improvement of retinal function in each case and the significance of any trend is yet to be established.

The data were examined by the ethical committee (GTAC) which is satisfied that there were no serious adverse events, and has given its approval for progression of the trial to the next phase.

### Discussion

For this first phase I/II clinical trial we included subjects who retained only limited residual retinal function. Despite advanced retinal degeneration we consistently measured, by both microperimetry and dark-adapted perimetry, unequivocal evidence of improved vision in one subject (subject #3). The difference in his performance in visual mobility in low light was also significantly greater than a modest learning effect and consistent with the improvement in visual function established by perimetry. It is not clear whether the improvement in visual responses in the peripheral macula was rod- or cone-mediated. Central macula function and visual acuity was not improved, despite exposure of this region to vector; this may be due either to ambylopia (the study eye was amblyopic) or a requirement for higher levels of RPE65 at the fovea. Visual function improved in only one subject (#3); he had better baseline visual acuity in both the study (amblyopic eye) and control eyes than either of the other subjects. Even though he was not the youngest subject, it is likely that he had less advanced retinal disease at baseline and this probably explains the improvement that was not observed in the other subjects. Whether further retinal degeneration is delayed in any of the subjects will become apparent only after several years.

The results of this study suggest that subretinal administration of rAAV vector is safe in humans with severe retinal dystrophy and AAV-mediated RPE65 gene therapy can lead to improved visual function, even in patients with advanced degeneration. This study supports the development of further clinical studies in children with RPE65 deficiency who are more likely to benefit.

What is claimed is:

1. A system for subretinal delivery of a vector to an eye of a human, comprising:
(a) a fine-bore cannula, wherein the fine bore cannula is 27 to 45 gauge;
(b) a syringe; and
(c) greater than about 0.8 ml of a suspension comprising an effective amount of the vector;
wherein the vector comprises a polynucleotide encoding a therapeutic polypeptide or therapeutic RNA under the control of a promoter suitable for expression of the therapeutic polypeptide or therapeutic RNA in one or more central retina cell types; and
wherein the vector is useful for treatment of an ocular disorder when administered to the subretinal space of the central retina of the eye.

2. The system of claim 1, wherein the suspension is contained within the syringe.

3. The system of claim 1, wherein the cannula is attached to the syringe.

4. The system of claim 2, wherein the syringe is an Accurus® system syringe.

5. The system of claim 1, further comprising an automated injection pump.

6. The system of claim 5, wherein the automated injection pump is activated by a foot pedal.

7. The system of claim 5, wherein the syringe is inserted into the automated injection pump.

8. The system of claim 1, comprising at least about 0.9 ml of the suspension.

9. The system of claim 1, comprising at least about 1.0 ml of the suspension.

10. The system of claim 1, comprising at least about 1.5 ml of the suspension.

11. The system of claim 1, comprising at least about 2.0 ml of the suspension.

12. The system of claim 1, comprising about 0.8 to about 3.0 ml of the suspension.

13. The system of claim 12, comprising about 0.8 to about 2.5 ml of the suspension.

14. The system of claim 13, comprising about 0.8 to about 2.0 ml of the suspension.

15. The system of claim 14, comprising about 0.8 to about 1.5 ml of the suspension.

16. The system of claim 15, comprising about 0.8 to about 1.0 ml of the suspension.

17. The system of claim 1, comprising about 1.0 to about 3.0 ml of the suspension.

18. The system of claim 17, comprising about 1.0 to about 2.0 ml of the suspension.

19. The system of claim 1, wherein the concentration of the vector in the suspension is about 1×10⁶ DRP/ml to about 1×10¹⁴ DRP/ml.

20. The system of claim 19, wherein the concentration of the vector in the suspension is about 1×10¹¹ DRP/ml.

21. The system of claim 20, wherein the suspension further comprises a therapeutic agent.

22. The system of claim 21, wherein the therapeutic agent is a neurotrophic factor, an anti-angiogenic factor, an anti-angiogenic polynucleotide, an anti-angiogenic morpholino, or an anti-angiogenic antibody or antigen-binding fragment thereof.

23. The system of claim 1, wherein the fine-bore cannula is 35-41 gauge.

24. The system of claim 23, wherein the fine-bore cannula is 40 or 41 gauge.

25. The system of claim 24, wherein the fine-bore cannula is 41-gauge.

26. The system of claim 1, wherein the vector is a recombinant adeno-associated virus (AAV) vector.

27. The system of claim 26, wherein the recombinant AAV vector is an AAV2, AAV4, AAV5, or AAV8 vector.

28. The system of claim 26, wherein the recombinant AAV vector is a pseudotyped AAV vector or chimeric AAV vector.

29. The system of claim 26, wherein the recombinant AAV vector comprises a mixture of AAV serotypes, pseudotypes, or chimeric vectors.

30. The system of claim 1, wherein the vector is selected from the group consisting of adenoviral, HSV, and lentiviral vectors.

31. The system of claim 30, wherein the vector is a lentiviral vector.

32. The system of claim 31, wherein the lentiviral vector is selected from the group consisting of HIV-1, HIV-2, SIV, FIV and EIAV.

33. The system of claim 1, wherein the polynucleotide is selected to replace a mutated gene known to cause retinal disease.

34. The system of claim 33, wherein the polynucleotide comprises a sequence encoding a polypeptide selected from the group consisting of: Prph2, RPE65, MERTK, RPGR, RP2, RPGRIP, CNGA3, CNGB3, and GNAT2.

35. The system of claim 34, wherein the polynucleotide is RPE65.

36. The system of claim 35, wherein the polynucleotide is hRPE65.

37. The system of claim 34, wherein the polynucleotide encodes the polypeptide RPE65.

38. The system of claim 37, wherein the polynucleotide encodes the polypeptide hRPE65.

39. The system of claim 1, wherein the polynucleotide comprises a sequence encoding a polypeptide selected from the group consisting of a neurotrophic factor, an anti-apoptotic factor, an anti-angiogenic factor, and an anti-inflammatory factor.

40. The system of claim 39, wherein the polynucleotide comprises a sequence encoding a polypeptide selected from the group consisting of GDNF, CNTF, FGF2, PEDF, EPO, BCL2, BCL-X, NFκB, Endostatin, Angiostatin, sFlt, IL10, IL1-ra, TGFβ, and IL4.

41. The system of claim 1, wherein the polynucleotide comprises a sequence encoding a therapeutic RNA.

42. The system of claim 36, wherein the polynucleotide is under the control of a promoter sequence, and the promoter sequence is hRPE65 promoter (hRPEp).

43. The system of claim 36, wherein the vector is AAV2/2.hRPE65p.hRPE65 (SEQ ID NO:1).

44. The system of claim 43, wherein the vector is useful for transducing retinal pigment epithelial cells.

45. The system of claim 44, wherein the vector is useful for transducing photoreceptor cells.

46. A system for subretinal delivery of a vector to an eye of a human, comprising:
(a) a fine-bore cannula, wherein the fine bore cannula is 27 to 45 gauge;
(b) a first syringe comprising a first fluid suitable for subretinal injection to the eye; and
(c) a second syringe comprising a second fluid comprising an effective amount of the vector;
wherein the total volume of the first and the second fluids in combination is about 0.5 ml to about 3.0 ml;
wherein the vector comprises a polynucleotide encoding a therapeutic polypeptide or therapeutic RNA under the control of a promoter suitable for expression of the therapeutic polypeptide or therapeutic RNA in one or more central retina cell types; and
wherein the vector is useful for treatment of an ocular disorder when administered to the subretinal space of the central retina of the eye.

47. The system of claim 46, wherein the first and second syringes are Accurus® system syringes.

48. The system of claim 46, further comprising an automated injection pump.

49. The system of claim 48, wherein the automated injection pump is activated by a foot pedal.

50. The system of claim 46, wherein the total volume of the first and the second fluids in combination is about 0.8 to about 3.0 ml.

51. The system of claim 50, wherein the total volume of the first and the second fluids in combination is about 0.9 to about 3.0 ml.

52. The system of claim 51, wherein the total volume of the first and the second fluids in combination is about 1.0 to about 3.0 ml.

53. The system of claim 46, wherein the volume of the first fluid is about 0.1 to about 0.5 ml.

54. The system of claim 46, wherein the volume of the second fluid is about 0.5 to about 3.0 ml.

55. The system of claim 54, wherein the volume of the second fluid is about 0.8 to about 3.0 ml.

56. The system of claim 55, wherein the volume of the second fluid is about 0.9 to about 3.0 ml.

57. The system of claim 56, wherein the volume of the second fluid is about 1.0 to about 3.0 ml.

58. The system of claim 46, wherein the concentration of the vector in the second fluid is about 1×10⁶ DRP/ml to about 1×10¹⁴ DRP/ml.

59. The system of claim 58, wherein the concentration of the vector in the second fluid is about 1×10¹¹ DRP/ml.

60. The system of claim 46, wherein the first or the second fluid further comprises a therapeutic agent.

61. The system of claim 60, wherein the therapeutic agent is a neurotrophic factor, an anti-angiogenic factor, an anti-angiogenic polynucleotide, an anti-angiogenic morpholino, or an anti-angiogenic antibody or antigen-binding fragment thereof.

62. The system of claim 46, wherein the first fluid is saline.

63. The system of claim 46, wherein the fine-bore cannula is 35-41 gauge.

64. The system of claim 63, wherein the fine-bore cannula is 40 or 41 gauge.

65. The system of claim 64, wherein the fine-bore cannula is 41-gauge.

66. The system of claim 46, wherein the vector is a recombinant adeno-associated virus (AAV) vector.

67. The system of claim 66, wherein the recombinant AAV vector is an AAV2, AAV4, AAV5, or AAV8 vector.

68. The system of claim 66, wherein the recombinant AAV vector is a pseudotyped AAV vector or chimeric AAV vector.

69. The system of claim 66, wherein the recombinant AAV vector comprises a mixture of AAV serotypes, pseudotypes, or chimeric vectors.

70. The system of claim 46, wherein the vector is selected from the group consisting of adenoviral, HSV, and lentiviral vectors.

71. The system of claim 70, wherein the vector is a lentiviral vector.

72. The system of claim 71, wherein the lentiviral vector is selected from the group consisting of HIV-1, HIV-2, SIV, FIV and EIAV.

73. The system of claim 46, wherein the polynucleotide is selected to replace a mutated gene known to cause retinal disease.

74. The system of claim 73, wherein the polynucleotide comprises a sequence encoding a polypeptide selected from the group consisting of: Prph2, RPE65, MERTK, RPGR, RP2, RPGRIP, CNGA3, CNGB3, and GNAT2.

75. The system of claim 74, wherein the polynucleotide is RPE65.

76. The system of claim 75, wherein the polynucleotide is hRPE65.

77. The system of claim 74, wherein the polynucleotide encodes the polypeptide RPE65.

78. The system of claim 77, wherein the polynucleotide encodes the polypeptide hRPE65.

79. The system of claim 46, wherein the polynucleotide comprises a sequence encoding a polypeptide selected from the group consisting of a neurotrophic factor, an anti-apoptotic factor, an anti-angiogenic factor, and an anti-inflammatory factor.

80. The system of claim 79, wherein the polynucleotide comprises a sequence encoding a polypeptide selected from the group consisting of GDNF, CNTF, FGF2, PEDF, EPO, BCL2, BCL-X, NFκB, Endostatin, Angiostatin, sFlt, IL10, IL1-ra, TGFβ, and IL4.

81. The system of claim 46, wherein the polynucleotide comprises a sequence encoding a therapeutic RNA.

82. The system of claim 76, wherein the polynucleotide is under the control of a promoter sequence, and the promoter sequence is hRPE65 promoter (hRPEp).

83. The system of claim 76, wherein the vector is AAV2/2.hRPE65p.hRPE65 (SEQ ID NO:1).

84. The system of claim 83, wherein the vector is useful for transducing retinal pigment epithelial cells.

85. The system of claim 83, wherein the vector is useful for transducing photoreceptor cells.

86. A method for treating an ocular disorder, comprising:
administering to the subretinal space of the central retina in an eye of a human in need thereof an effective amount of a vector;
wherein the vector is useful for treatment of the ocular disorder when administered to the subretinal space of the central retina of the eye.

87. The method of claim 86, wherein the vector comprises a polynucleotide encoding a therapeutic polypeptide or therapeutic RNA; and wherein the polynucleotide is under the control of a promoter suitable for expression of the therapeutic polypeptide or therapeutic RNA in one or more central retina cell types.

88. The method of claim 87, wherein one or more cells in contact with the subretinal space of the central retina are transduced by the vector and express the therapeutic polypeptide or therapeutic RNA encoded by the polynucleotide.

89. The method of claim 87, wherein one or more cells in contact with the subretinal space of the outer macula are transduced by the vector and express the therapeutic polypeptide or therapeutic RNA encoded by the polynucleotide.

90. The method of claim 87, wherein one or more cells in contact with the subretinal space of the inner macula are transduced by the vector and express the therapeutic polypeptide or therapeutic RNA encoded by the polynucleotide.

91. The method of claim 87, wherein one or more cells in contact with the subretinal space of the fovea are transduced by the vector and express the therapeutic polypeptide or therapeutic RNA encoded by the polynucleotide.

92. The method of claim 87, wherein the one or more cells are retinal pigment epithelial cells.

93. The method of claim 87, wherein the one or more cells are photoreceptor cells.

94. The method of claim 87, wherein the polynucleotide comprises a sequence encoding a therapeutic polypeptide.

95. The method of claim 87, wherein the polynucleotide comprises a sequence encoding a therapeutic RNA.

96. The method of claim 86, wherein the vector is administered to the outer macula.

97. The method of claim 96, wherein the vector is administered to the inner macula.

98. The method of claim 97, wherein the vector is administered to the fovea.

99. The method of claim 98, wherein the method does not significantly adversely affect central retinal function or central retinal structure.

100. The method of claim 99, wherein the ocular disorder is selected from the group consisting of: autosomal recessive severe early-onset retinal degeneration (Leber's Congenital Amaurosis), congenital achromatopsia, Stargardt's disease, Best's disease, Doyne's disease, cone dystrophy, retinitis pigmentosa, X-linked retinoschisis, Usher's syndrome, atrophic age related macular degeneration, neovascular AMD, diabetic maculopathy, proliferative diabetic retinopathy (PDR), cystoid macular oedema, central serous retinopathy, retinal detachment, intra-ocular inflammation, and posterior uveitis.

101. The method of claim 100, wherein the ocular disorder is autosomal recessive severe early-onset retinal degeneration (Leber's Congenital Amaurosis).

102. The method of claim 101, wherein the method is effective in treating the human's visual function.

103. The method of claim 102, wherein visual function is assessed by microperimetry, dark-adapted perimetry, assessment of visual mobility, visual acuity, ERG, or reading assessment.

104. The method of claim 102, wherein visual function is assessed by microperimetry, dark-adapted perimetry, or assessment of visual mobility.

105. The method of claim 102, wherein the method results in an improvement in the human's visual function.

106. The method of claim 102, wherein the method results in the prevention of or a slowing of the progression of decline of the human's visual function due to progression of the ocular disorder.

107. The method of claim 86, wherein the vector is a recombinant adeno-associated virus (AAV) vector.

108. The method of claim 107, wherein the recombinant AAV vector is an AAV2, AAV4, AAV5, or AAV8 vector.

109. The method of claim 107, wherein the recombinant AAV vector is a pseudotyped AAV vector or chimeric AAV vector.

110. The method of claim 107, wherein the recombinant AAV vector comprises a mixture of AAV serotypes, pseudotypes, or chimeric vectors.

111. The method of claim 87, wherein the polynucleotide is selected to replace a mutated gene known to cause retinal disease.

112. The method of claim 111, wherein the polynucleotide encodes a sequence encoding a polypeptide selected from the group consisting of: Prph2, RPE65, MERTK, RPGR, RP2, RPGRIP, CNGA3, CNGB3, and GNAT2.

113. The method of claim 112, wherein the polynucleotide is RPE65.

114. The method of claim 113, wherein the polynucleotide is hRPE65.

115. The method of claim 112, wherein the polynucleotide encodes the polypeptide RPE65.

116. The method of claim 115, wherein the polynucleotide encodes the polypeptide hRPE65.

117. The method of claim 87, wherein the polynucleotide comprises a sequence encoding a polypeptide selected from the group consisting of a neurotrophic factor, an anti-apoptotic factor, an anti-angiogenic factor, and an anti-inflammatory factor.

118. The method of claim 117, wherein the polynucleotide comprises a sequence encoding a polypeptide selected from the group consisting of GDNF, CNTF, FGF2, PEDF, EPO, BCL2, BCL-X, NFκB, Endostatin, Angiostatin, sFlt, IL10, IL1-ra, TGFβ, and IL4.

119. The method of claim 87, wherein the polynucleotide comprises a sequence encoding a therapeutic RNA.

120. The method of claim 114, wherein the polynucleotide is under the control of a promoter sequence, and the promoter sequence is hRPE65 promoter (hRPEp).

121. The method of claim 114, wherein the vector is AAV2/2.hRPE65p.hRPE65 (SEQ ID NO:1).

122. The method of claim 87, further comprising administering a therapeutic agent to the subretinal space of the central retina of the eye.

123. The method of claim 122, wherein the therapeutic agent is a neurotrophic factor, an anti-angiogenic factor, an anti-angiogenic polynucleotide, an anti-angiogenic morpholino, or an anti-angiogenic antibody or antigen-binding fragment thereof.

124. The method of claim 87, comprising administering to the human about 0.5 ml to about 3.0 ml of a suspension comprising the vector.

125. The method of claim 124, comprising administering to the human about 0.8 ml to about 3.0 ml of a suspension comprising the vector.

126. The method of claim 125, comprising administering to the human about 0.9 ml to about 3.0 ml of a suspension comprising the vector.

127. A kit comprising the system of claim 1, and instructions for use.

128. The kit of claim 127, wherein the instructions for use comprise instructions for performing a method for treating an ocular disorder comprising:
administering to the subretinal space of the central retina in an eye of a human in need thereof an effective amount of a vector;
wherein the vector is useful for treatment of the ocular disorder when administered to the subretinal space of the central retina of the eye.

129. A kit comprising the system of claim 46, and instructions for use.

130. The kit of claim 129, wherein the instructions for use comprise instructions for performing a method for treating an ocular disorder comprising:
administering to the subretinal space of the central retina in an eye of a human in need thereof an effective amount of a vector;
wherein the vector is useful for treatment of the ocular disorder when administered to the subretinal space of the central retina of the eye.


SEQUENCE LISTING (1 sequence)

SEQ ID NO: 1 - DNA, 3547 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactgatgtaatacgactcactagtgattctctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtcctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttggtaatcgaattcatgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaatgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaagtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaagcttcgagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctagtcaaggatcagtgatggagttggccactccctctctgcgcgctcgctcgctcactgaggccgggcgaccaaaggtcgcccgacgcccgggctttgcccgggcggcctcagtgagcgagcgagcgcgcag